SEQUENCE LISTING (23 sequences)

SEQ ID NO: 1 - skipped

SEQ ID NO: 2 - protein, 15 aa, synthetic construct
LLPPTEQDLTALALA

SEQ ID NO: 3 - protein, 15 aa, synthetic construct
LLPPTEQDLAKLAAY

SEQ ID NO: 4 - protein, 15 aa, synthetic construct
LLPPTAQDLAKLALY

SEQ ID NO: 5 - protein, 15 aa, synthetic construct
LLPPTEADLTALALY

SEQ ID NO: 6 - protein, 15 aa, synthetic construct
LLPPTEQDLTCLALC

SEQ ID NO: 7 - protein, 15 aa, synthetic construct
LLPPTEQDLCKLACY

SEQ ID NO: 8 - protein, 15 aa, synthetic construct
LLPPTCQDLCKLALY

SEQ ID NO: 9 - protein, 15 aa, synthetic construct
LLPPTECDLTCLALY

SEQ ID NO: 10 - protein, 15 aa, synthetic construct
LLPPTEQDLXKLAXY

SEQ ID NO: 11 - protein, 15 aa, synthetic construct
LLPPTEQDLTXLALX

SEQ ID NO: 12 - protein, 15 aa, synthetic construct
LLPPTEADLAKLAAY

SEQ ID NO: 13 - protein, 15 aa, synthetic construct
LLPPTEQDLAKLAAY

SEQ ID NO: 14 - protein, 15 aa, synthetic construct
LLPPTEQDLAALAAY

SEQ ID NO: 15 - protein, 15 aa, synthetic construct
LLPPTEQDLCKLACY

SEQ ID NO: 16 - skipped

SEQ ID NO: 17 - protein, 4 aa, synthetic construct
RLAA

SEQ ID NO: 18 - DNA, 24 bp, synthetic construct
  source 1..24: mol_type = other DNA
gtttgtagaccaggaacagttgaa

SEQ ID NO: 19 - DNA, 20 bp, synthetic construct
  source 1..20: mol_type = other DNA
cgcatggtgtcctccattat

SEQ ID NO: 20 - DNA, 20 bp, synthetic construct
  source 1..20: mol_type = other DNA
tgctgcaggtctcctcttgg

SEQ ID NO: 21 - DNA, 20 bp, synthetic construct
  source 1..20: mol_type = other DNA
agtcccaggcgtagaccaag

SEQ ID NO: 22 - DNA, 20 bp, synthetic construct
  source 1..20: mol_type = other DNA
gaggccgtaggcttattgtg

SEQ ID NO: 23 - DNA, 26 bp, synthetic construct
  source 1..26: mol_type = other DNA
gagtagctcatatgtcttccctacct

(19) United States
(12) Patent Application Publication
(10) Pub. No.: US 2023/0405134 A1
(43) Pub. Date: Dec. 21, 2023

(54) NOVEL PROTAC CHIMERIC COMPOUND, AND PHARMACEUTICAL COMPOSITION COMPRISING SAME FOR PREVENTING, AMELIORATING, OR TREATING DISEASES THROUGH TARGET PROTEIN DEGRADATION

(71) Applicant: POSTECH RESEARCH AND BUSINESS DEVELOPMENT FOUNDATION, Pohang-si (KR)

(72) Inventors: Hyun Suk LIM, Pohang-si (KR); Yeong Ju LEE, Pohang-si (KR)

(21) Appl. No.: 18/148,653

(22) Filed: Dec. 30, 2022

Related U.S. Application Data
(63) Continuation-in-part of application No. PCT/KR2021/008437, filed on Jul. 2, 2021.

(30) Foreign Application Priority Data
Jul. 2, 2020 (KR) 10-2020-0081373

(51) Int. Cl. A61K 47/62; A61K 47/54; A61K 47/65; A61P 35/00

(52) U.S. Cl. CPC A61K 47/62; A61K 47/545; A61P 35/00; A61K 47/54; A61K 47/65

(57) ABSTRACT

The present invention relates to a novel class of a chimeric molecule associated with a chimeric compound design for degrading a desired target protein, SRC-1. More specifically, the present invention relates to a peptide compound for degrading SRC-1 protein, and a pharmaceutical composition for preventing or treating cancer metastasis and occurrence caused by SRC-1 overexpression, and for preventing or treating immune-related diseases.

### REFERENCE TO AN ELECTRONIC SEQUENCE LISTING

The electronic sequence listing (EPL20226067US_Sequence_revised.xml; Size: 20 K bytes; and Date of Creation: Mar. 15, 2023) is herein incorporated by reference in its entirety.

### TECHNICAL FIELD

The present invention relates to a PROTAC chimeric compound in which a novel E3 ligase ligand and a target protein ligand are linked by a linker, a stereoisomer, solvate or hydrate thereof, and a pharmaceutical composition or food composition for preventing or treating diseases through target protein degradation, including the same. More specifically, the present invention relates to a novel PROTAC chimeric compound in which a novel E3 ligase ligand and a ligand of target protein, SRC-1, are linked by a linker, wherein the novel PROTAC chimeric compound specifically binds to SRC-1, and a E3 ligase binding to the E3 ligase ligand selectively degrades and removes SRC-1, and pharmaceutical compositions or food compositions for preventing, ameliorating or treating immune-related diseases and cancers caused by overexpression of SRC-1.

### BACKGROUND ART

PROTAC is a heterodimeric molecule in which a ligand of a target protein is connected via a linker to a ligand binding to an E3 ligase. The PROTAC binds to both proteins simultaneously, bringing the target protein into close proximity to the E3 ligase, whereby the E3 ligase recognizes the target protein as a substrate, resulting in polyubiquitination and subsequent proteasomal degradation. Since a specific protein can be effectively removed from cells using this principle, the PROTAC can be used as a chemical probe to study the function of the target protein and further has high potential as a therapeutic agent for disease. However, despite these advantages, there are currently limitations in using PROTAC technology. One of the limitations is that the types of cells and tissues which can be targeted are limited. More than 600 E3 ubiquitin ligases exist in the human body, but only a few E3 ubiquitin ligases, such as cereblon (CRBN) and Von Hippel-Lindau tumor suppressor (VHL), are currently used as the E3 ligase in the PROTAC design. In this case, the proteolytic effects cannot be exerted in cells or tissues in which the E3 ligase targeted by the PROTAC molecule developed according to the conventional method is not sufficiently expressed.

An N-degron pathway is a system that recognizes amino acids at the N-terminus of a substrate protein and degrades the protein. The N-terminal amino acids of the substrate protein are recognized by the UBR E3 ubiquitin ligase family, and the substrate protein undergoes ubiquitination and degradation by the proteasome. Thus, the N-degron pathway determines the fate of the target protein in cells according to the type of the N-terminal amino acids of the substrate protein. Since the UBR protein is expressed non-ubiquitously and highly in most cells, a desired target protein can be efficiently degraded regardless of cell type by using the PROTAC composed of UBR protein ligands.

Steroid receptor coactivator-1 (SRC-1, also known as NCOA-1) is a transcriptional coactivator that promotes the transcriptional activity of various transcription factors (TFs) such as estrogen receptor a and progesterone receptor. The SRC-1 belongs to a p160 SRC family, which includes other homologous members such as SRC-2 (NCOA-2) and SRC-3 (NCOA-3). It contains four domains, including AD3 located in the activation domain at the N-terminus, a nuclear receptor interaction domain, and AD1 and AD2 located at the C-terminus. The SRC-1 not only interacts with transcription factors as a transcriptional coactivator, but also interacts with various other proteins to form multi-protein complexes. Consequently, The SRC-1 plays an important role in inducing chromatin remodeling and transcriptional activation and regulating various biological signals such as metabolism and inflammation. However, abnormally elevated SRC-1 expression and activity have been reported to be involved in cancer metastasis, recurrence, drug resistance and poor prognosis. Therefore, inhibition of SRC-1 can be an effective therapeutic strategy for the treatment of various cancers. However, it is very difficult to develop a molecule that regulates the SRC-1 transcriptional activity because protein-protein interactions must be targeted. Several SRC-1 inhibitors have been developed so far, but have problems in that they have very low activity or no selectivity for SRC-1. As a representative example, a low molecular weight compound developed by the O'Malley group exhibited the effect of effectively inhibiting SRC-1 in cells and mouse experiments. However, the compound has no selectivity for SRC-1, and its mechanism of action is also unclear. Therefore, there is an urgent need to discover an SRC-1 inhibitor with selectivity and high activity.

### DISCLOSURE

### Technical Problem

The present invention has been conceived to solve the above problems, and an object of the present invention is to provide a compound that binds to and degrades an SRC-1 protein.

Another object of the present invention is to provide a use of the compound degrading an SRC-1 protein for preventing or treating immune-related diseases and/or cancers caused by SRC-1 overexpression, and/or for suppressing cancer metastasis.

### Technical Solution

In order to achieve the above object, the present invention provides a chimeric compound having a structure of Formula 1 below:

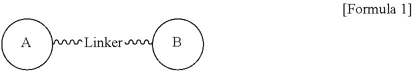

wherein:
A represents a ubiquitin ligase binding moiety (ULM) that binds to any one or more E3 ubiquitin ligases selected from the group consisting of ubiquitin-protein ligase E3 component n-recognin 1 (UBR1), ubiquitin-protein ligase E3 component n-recognin 2 (UBR2) and ubiquitin-protein ligase E3 component n-recognin 4 (UBR4), and
B represents a protein target moiety (PTM) that binds to steroid receptor coactivator-1 (SRC-1), wherein A and B are chemically linked by a linker.

According to another preferred embodiment of the present invention, wherein the chimeric compound binds simultaneously to protein and ubiquitin ligase, the protein may be ubiquitinated by the ubiquitin ligase. According to another preferred embodiment of the present invention, the linker may have a structure of Formula 2:

—Y₁—Y₂—Y₃— [Formula 2]
wherein Y₁is R₁, or Y₁ is absent;
R₁ is selected from the group consisting of —C(═O)N(H)—, —N(H)—, —N(H)C(═O)—, —O—, —CH₂—, —CH═CH— and —C≡C—;
Y₂ is any one selected from the group consisting of —C(═O)N(H)—, —N(H)—, —N(H)C(═O)—, —O—, —CH₂—, —CH═CH— and —C≡C—; and
Y₃ is selected from the group consisting of —C(═O)—, —N(H)—, —C(═O)N(H)—, —N(H)C(═O)—, —O—, —CH₂—, —CH═CH— —C≡C— and or absent.

According to another preferred embodiment of the present invention, Y₂ is selected from the group consisting of —CH₂ (CH₂ OCH₂)m1CH₂—, —(CH₂)m2—W—(CH₂)m3—, —(CH₂)m2—W—(CH₂)m4—O—(CH₂)m5— and —(N(H)CH(CH₃)C(═O))m6—;
W is selected from the group consisting of phenylene, five-membered heteroarene and cycloalkylene, or absent;
m1 is 1, 2, 3, 4, 5, 6 or 7;
m2 is 0, 1, 2, 3, 4, 5, 6 or 7;
m3 is 0, 1, 2, 3, 4, 5, 6 or 7;
m4 is 0, 1, 2, 3 or 4;
m5 is 0, 1, 2, 3 or 4; and
m6 is 0, 1, 2, 3 or 4.

According to another preferred embodiment of the present invention, the protein target moiety (PTM) comprises an amino acid sequence of X₁X₂X₃X₄X₅X₆X₇X₈X₉X₁₀X₁₁X₁₂X₁₃X₁₄X₁₅ (SEQ ID NO: 1),
wherein the amino acid sequence of SEQ ID NO: 1 is a stapled peptide in which two amino acids in the amino acid sequence of SEQ ID NO: 1 are linked to each other, and
in the amino acid sequence,
X₁, X₂, X₉ and X₁₂ may be valine (V), alanine (A), isoleucine (I), leucine (L), norleucine (Nleu), 3-methyl valine or norvaline;
X₃ and X₄ may be proline (P), hydroxy proline, amino proline, propynyl proline, chloro proline, bromo proline or trifluoromethyl proline;
X₅ may be threonine (T), serine (S), homoserine, methyl homoserine, or alanine (A);
X₆ may be glutamic acid (E), aspartic acid (D), or alanine (A);
X₇ may be glutamine (Q), asparagine (N), or alanine (A);
X₈ may be glutamic acid (E) or aspartic acid (D);
X₁₀ may be (S)-2-(4′-pentenyl) alanine, cysteine (C), homocysteine, lysine (K), ornithine (Orn) or diaminobutyric acid (Dab);
X₁₁ may be arginine (R), lysine (K) or alanine (A);
X₁₂ may be leucine (L) or alanine (A);
X₁₃ may be cyclohexylalanine (Cha), cyclopentylalanine (Cpa), cycloheptylpropanoic acid, phenylalanine (F), leucine (L), alanine (A), isoleucine (I) or valine (V);
X₁₄ may be (S)-2-(4′-pentenyl) alanine, cysteine (C), homocysteine, lysine (K), ornithine (Orn) or diaminobutyric acid (Dab); and
X₁₅ may be tyrosine (Y), serine (S), threonine (T) or alanine (A).

According to another preferred embodiment of the present invention, two amino acids functionalized with a compound containing the (S)-2-(4′-pentenyl) alanine group can be linked by a ring produced through ring-closing metathesis, or can be so linked and then linked by a carbon-carbon single bond through a reduction reaction.

According to another preferred embodiment of the present invention, wherein two amino acids in the amino acid sequence of SEQ ID NO: 1 are X₁₀ and X₁₄ and the two amino acids are cysteine or homocysteine, respectively, they may be linked by cyclization with a compound comprising a phenyl group.

According to another preferred embodiment of the present invention, the compound comprising a phenyl group may be represented by Formula 3 or Formula 4 below:

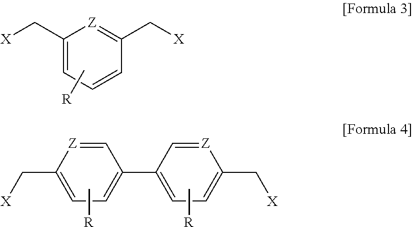

wherein X is at least one selected from the group consisting of chloro, bromo, and iodo; Z is nitrogen or oxygen; and R is at least one selected from the group consisting of hydrogen, halogen, C₁₋₄ alkyl, C₁₋₄ alkyl substituted with halogen, nitro, amino, and C₁₋₄ alkylamino.

According to another preferred embodiment of the present invention, wherein two amino acids in the amino acid sequence of SEQ ID NO: 1 are X₁₀ and X₁₄ and the two amino acids are lysine (K) and ornithine (Orn) or diaminobutyric acid (Dab), respectively, they may be linked by cyclization with a compound comprising triazine.

According to another preferred embodiment of the present invention, a linker may be coupled to the N-terminus or C-terminus of SEQ ID NO: 1.

According to another preferred embodiment of the present invention, the ubiquitin ligase binding moiety (ULM) may include an amino acid sequence of X₂₀X₂₁X₂₂X₂₃ (SEQ ID NO: 16).

According to another preferred embodiment of the present invention, X₂₀ may be arginine (R), histidine (H), lysine (K), phenylalanine (F), tyrosine (Y), isoleucine (I), tryptophan (W), glutamic acid (E) or aspartic acid (D);
X₂₁ may be arginine (R), leucine (L), isoleucine (I), alanine (A), valine (V), glycine (G) or phenylalanine (F), or absent;
X₂₂ and X₂₃ may be alanine (A), glycine (G) or valine (V), or absent.

According to another preferred embodiment of the present invention, the compound may include one or more selected from the group consisting of a plurality of ULMs, a plurality of PTMs, and a plurality of linkers.

According to another preferred embodiment of the present invention, Formula 1 may be any one formula selected from the group consisting of Formulas 5 to 16 below:

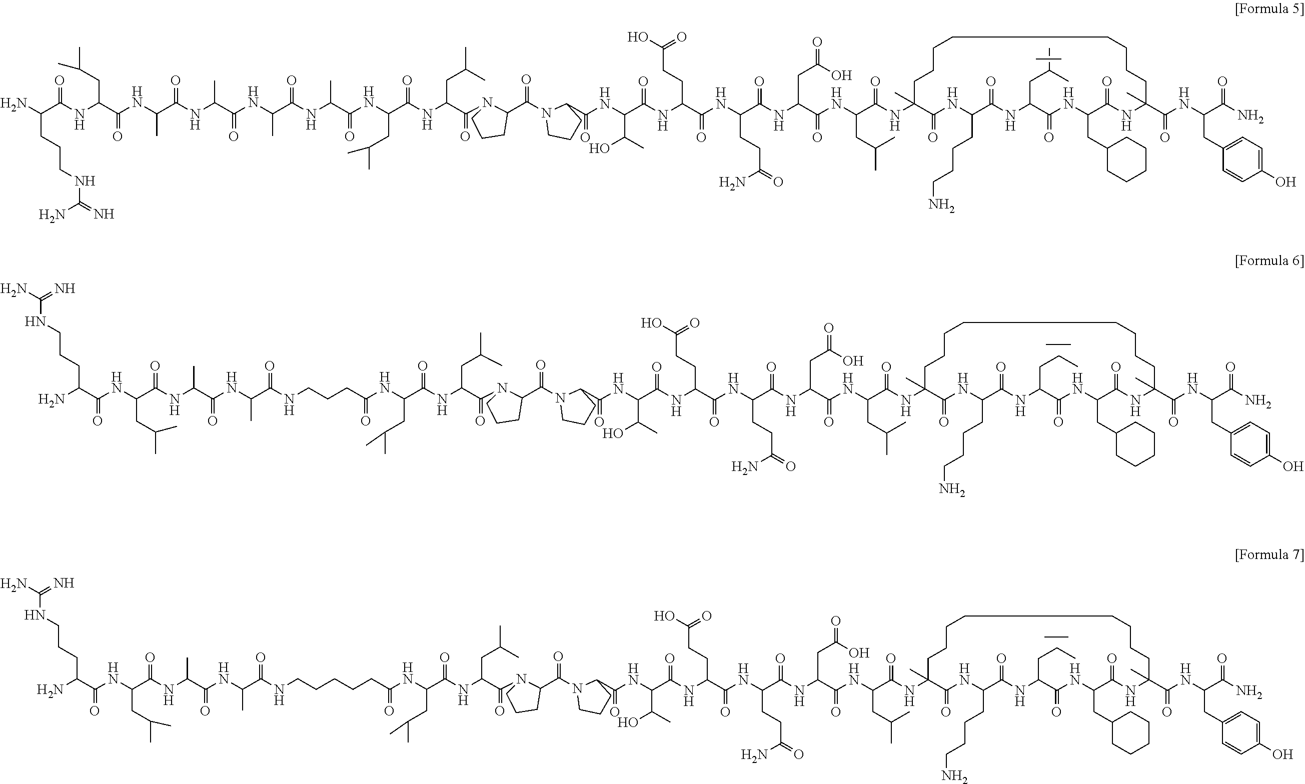

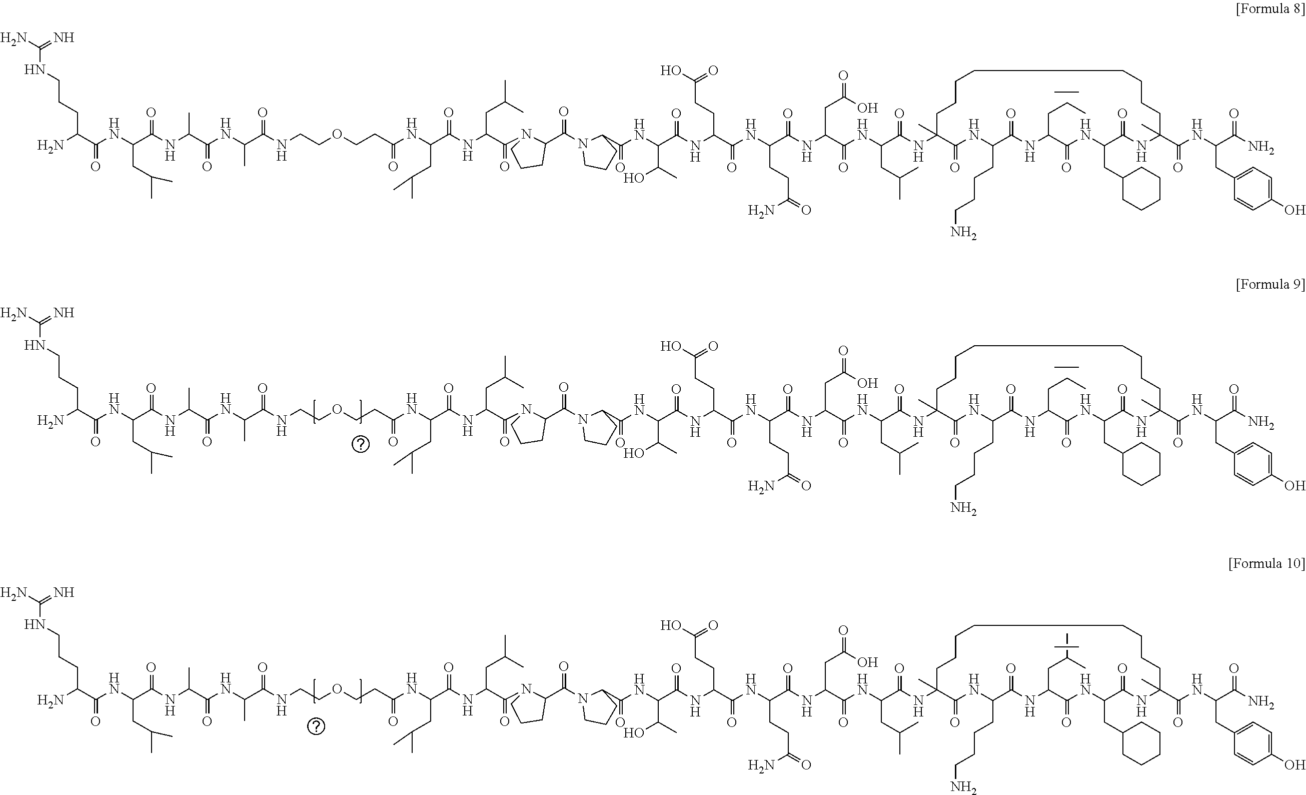

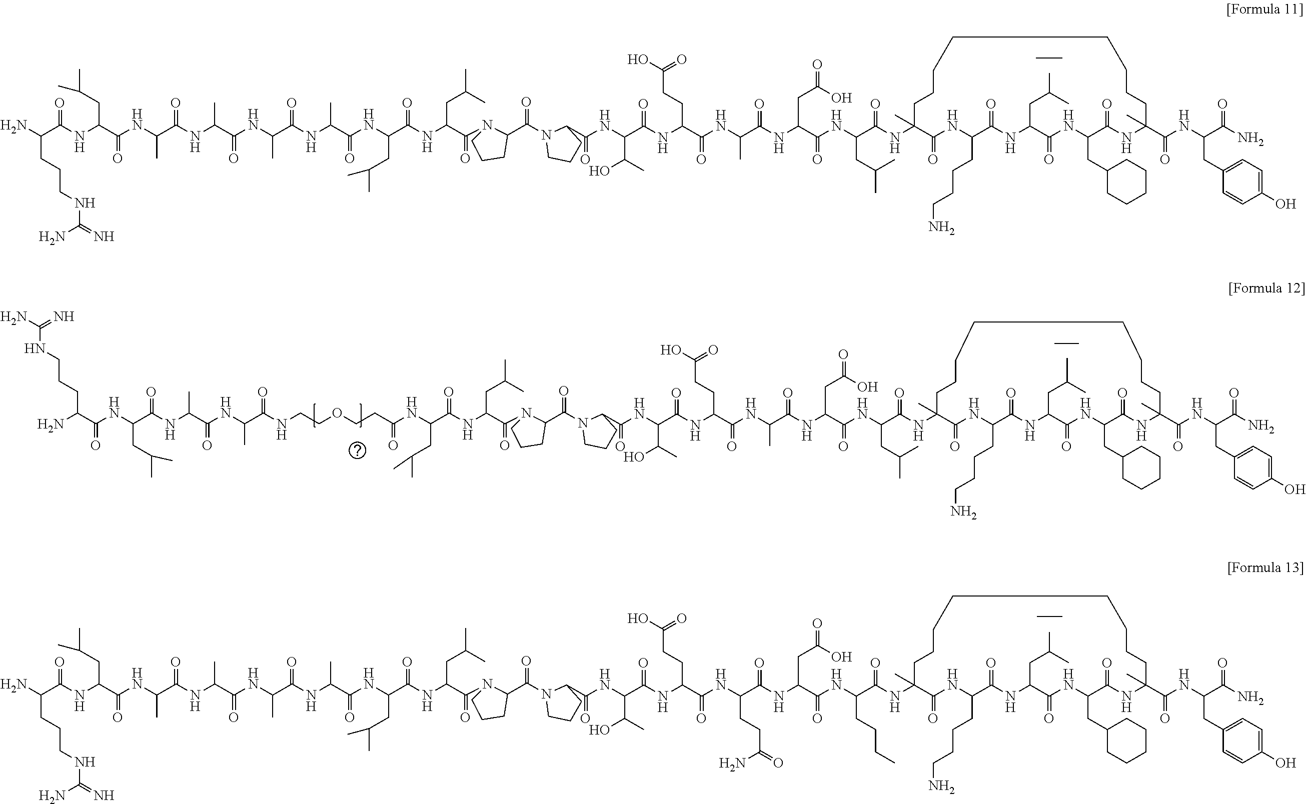

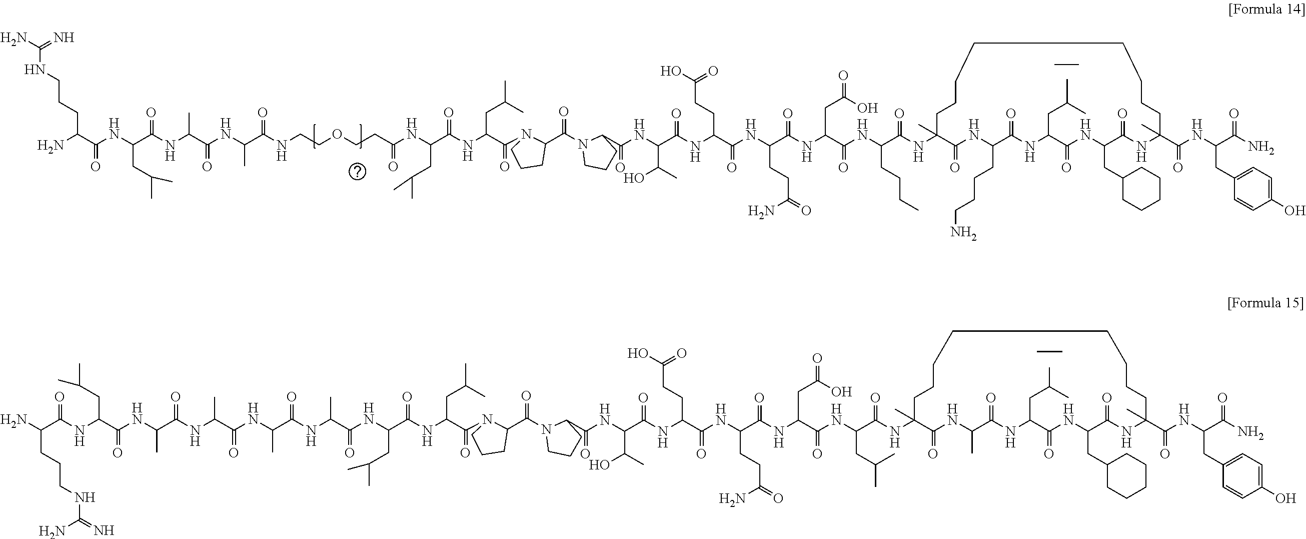

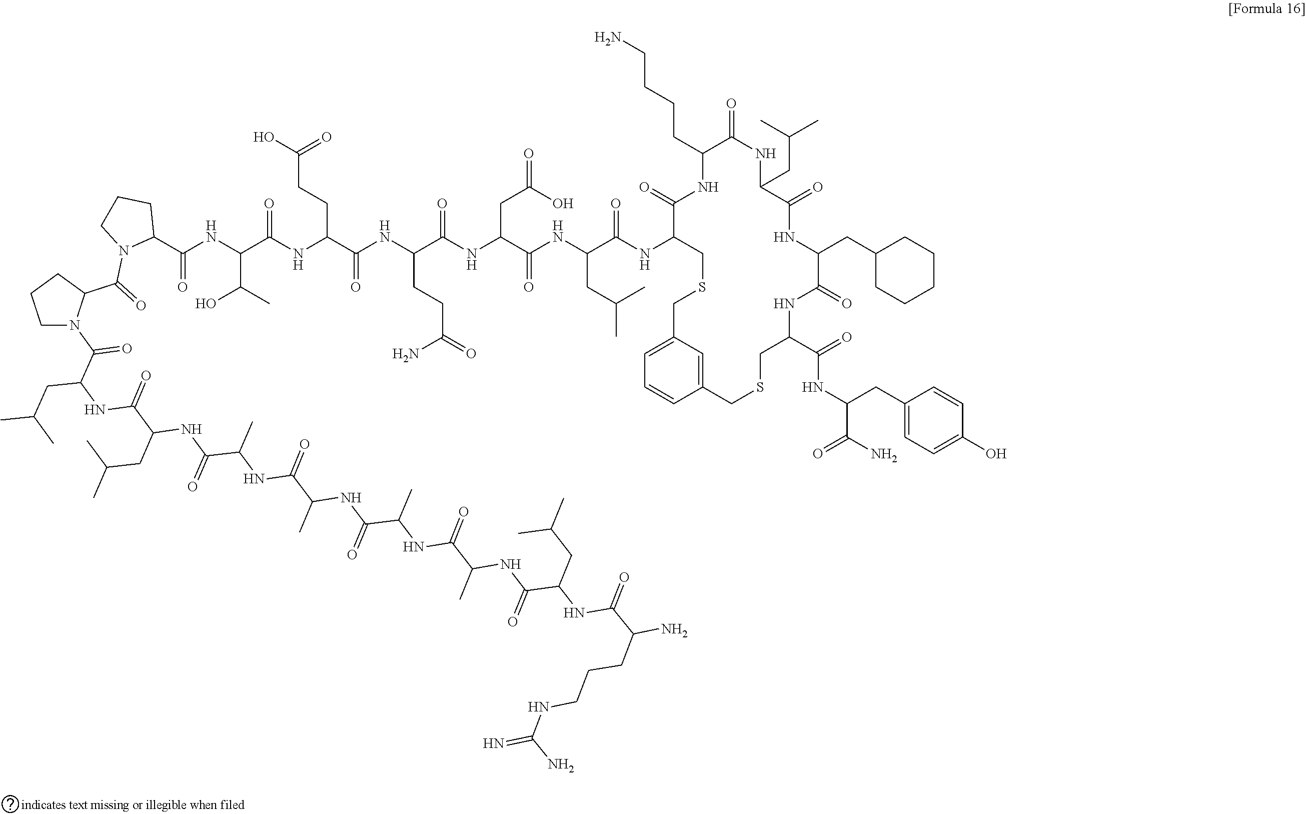 [SMILES: CC(C)CC(NC(=O)C(N)CCCNC(=N)N)C(=O)NC(C)C(=O)NC(C)C(=O)NC(C)C(=O)NC(C)C(=O)NC(CC(C)C)C(=O)NC(CC(C)C)C(=O)N1CCCC1C(=O)N1CCCC1C(=O)NC(C(=O)NC(CCC(=O)O)C(=O)NC(CCC(N)=O)C(=O)NC(CC(=O)O)C(=O)NC(CC(C)C)C(=O)NC1CSCc2cccc(c2)CSCC(C(=O)NC(Cc2ccc(O)cc2)C(N)=O)NC(=O)C(CC2CCCCC2)NC(=O)C(CC(C)C)NC(=O)C(CCCCN)NC1=O)C(C)O]

The present invention also provides a method for preventing or treating diseases caused by overexpression of SRC-1, the method comprising administering any one of the chimeric compounds described above, isomers, solvates or hydrates thereof and/or a composition comprising any one of the chimeric compounds described above, isomers, solvates or hydrates thereof to a subject in need thereof.

In the present invention, the disease may be:
any one or more immune-related diseases selected from the group consisting of atopic dermatitis, asthma, airway hypersensitivity and chronic obstructive pulmonary disease;
any one or more cancers selected from the group consisting of breast cancer, prostate cancer, skin melanoma, thyroid cancer, and endometrial cancer; or
metastasis of the cancer.

The present invention also provides a method of inhibiting metastasis of cancer, the method comprising administering any one of the chimeric compounds described above, isomers, solvates or hydrates thereof and/or a composition comprising any one of the chimeric compounds described above, isomers, solvates or hydrates thereof to a subject in need thereof.

According to another preferred embodiment of the present invention, the cancer may be any one selected from the group consisting of breast cancer, prostate cancer, skin melanoma, thyroid cancer, and endometrial cancer.

### Advantageous Effects

The compounds of the present invention specifically bind to SRC-1, and the E3 ligase binding to the E3 ligase ligand degrades and selectively removes SRC-1, thereby exhibiting the effect of preventing or treating immune-related diseases and cancers, which are diseases caused by overexpression of SRC-1. Furthermore, the compounds of the present invention can overcome the disadvantages of the conventional non-specific inhibitors, become drug candidates for various diseases such as breast cancer and prostate cancer caused by overexpression of SRC-1, and also serve as chemical probes capable of identifying a role of SRC-1 in the human body. The compounds of the present invention use an N-degron that recognizes and degrades an N-terminus of a substrate protein by the UBR E3 ubiquitin ligase family. By using the UBR E3 ubiquitin ligase family, which is expressed non-ubiquitously, there is an effect of degrading the target protein in cells that cannot be targeted by the existing PROTAC technology for degrading the target protein.

### DESCRIPTION OF DRAWINGS

FIG. 1 is a schematic diagram of SRC-1 degradation by PROTAC via an N-degron pathway.

FIG. 2 shows a chemical structure of YL2 peptide reported as a ligand of SRC-1.

FIG. 3 shows a crystal structure of SRC-1 and a ligand YL2.

FIGS. 4A and 4B show the chemical structure and nomenclature of synthetic compounds.

FIGS. 5A, 5B, and 5C illustrate a method of synthesizing compounds that cause degradation via an N-degron pathway. (ND1-YL2-ND1-YL6)

FIG. 6A shows an ND1-YL2 structure, and LC and MS data.

FIG. 6B shows an ND2-YL2 structure, and LC and MS data.

FIG. 6C shows an ND3-YL2 structure, and LC and MS data.

FIG. 6D shows an ND4-YL2 structure, and LC and MS data.

FIG. 6E shows an ND5-YL2 structure, and LC and MS data.

FIG. 6F shows an ND6-YL2 structure, and LC and MS data.

FIG. 6G shows an ND1-YLD6 structure, and LC and MS data.

FIG. 6H shows an ND5-YLD6 structure, and LC and MS data.

FIG. 6I shows an ND1-YLD12 structure, and LC and MS data.

FIG. 6J shows an ND5-YLD12 structure, and LC and MS data.

FIG. 6K shows an ND1-YLD13 structure, and LC and MS data.

FIG. 6L shows an ND1-YL6 structure, and LC and MS data.

FIG. 7A shows the result of analyzing an SRC-1 expression level in MDA-MB-231 cells after ND2-YL2 treatment through Western blot.

FIG. 7B shows the result of analyzing an SRC-1 expression level in MDA-MB-231 cells after ND3-YL2 treatment through Western blot.

FIG. 7C shows the result of analyzing an SRC-1 expression level in MDA-MB-231 cells after ND4-YL2 treatment through Western blot.

FIG. 7D shows the result of analyzing an SRC-1 expression level in MDA-MB-231 cells after ND5-YL2 treatment through Western blot.

FIG. 7E shows the result of analyzing an SRC-1 expression level in MDA-MB-231 cells after ND6-YL2 treatment through Western blot.

FIG. 7F shows the result of analyzing an SRC-1 expression level in MDA-MB-231 cells after ND1-YLD6 treatment through Western blot.

FIG. 7G shows the result of analyzing an SRC-1 expression level in MDA-MB-231 cells after ND5-YLD6 treatment through Western blot.

FIG. 7H shows the result of analyzing an SRC-1 expression level in MDA-MB-231 cells after ND1-YLD12 treatment through Western blot.

FIG. 7I shows the result of analyzing an SRC-1 expression level in MDA-MB-231 cells after ND5-YLD12 treatment through Western blot.

FIG. 7J shows the result of analyzing an SRC-1 expression level in MDA-MB-231 cells after ND1-YLD13 treatment through Western blot.

FIG. 7K shows the result of analyzing an SRC-1 expression level in MDA-MB-231 cells after ND1-YL6 treatment through Western blot.

FIG. 8 schematically illustrates a chemical structure of ND1-YL2.

FIG. 9A shows the result of analyzing an SRC-1 expression level in MDA-MB-231 cells, which are breast cancer cells, after ND1-YL2 or MG132 (5 μM) treatment for 12 hours through Western blot; FIG. 9B shows western blot analysis of an SRC-1 expression level in MDA-MB-231 cells after ND1-YL2 (20 μM) treatment for various treatment times; and FIG. 9C is a Western blot analysis image of an SRC-1 expression level in MDA-MB-231 cells over time after removal of the treated ND1-YL2 (20 μM).

FIG. 10 is circular dichroism (CD) spectra.

FIGS. 11A and 11B show the experimental results of the binding affinities of ND1-YL2 to YL2 to SRC-1 and UBR1 through analysis of fluorescence polarization (FP) assay. FIG. 11A is a curve showing a ND1-YL2 or YL2 to competitively bind to SRC-1 after binding of a fluorescently-labeled STAT-6 peptide; and FIG. 11B is a curve showing a ND1-YL2 to competitively bind to UBR-1 after binding of a fluorescently-labeled RLAA peptide.

FIG. 12A is data confirming the formation of an SRC-1-UBR box-ND1-YL2 complex using SEC-MALS analysis, indicating SRC-1 (blue), UBR box (purple) and SRC-1-UBR box-ND1-YL2 complex (red); FIG. 12B is an image of a solution SAXS structure of the SRC-1-UBR box-ND1-YL2 complex; and FIG. 12C is an image obtained by Western blot analysis of an SRC-1 expression level in MDA-MB-231 cells after treatment with ND1-YL2 (20 μM), YL2 (20 μM) to RLAA peptide (20 μM).

FIG. 13 illustrates a method of synthesizing CL1-YL2-CL3-YL2 compounds.

FIG. 14A shows an CL1-YL2 structure, and LC and MS data.

FIG. 14B shows an CL2-YL2 structure, and LC and MS data.

FIG. 14C shows an CL3-YL2 structure, and LC and MS data.

FIG. 15 shows the result of Western blot analysis of an SRC-1 expression level in MDA-MB-231 cells after CL1-YL2-CL3-YL2 treatment.

FIG. 16A illustrates a structure of CL1-YL2; FIG. 16B is the result of Western blot analysis of an SRC-1 expression level in A549 cells after treatment with CL1-YL2 (20 μM), and FIG. 16C is an image of Western blot analysis of an SRC-1 expression level in Colo205 cells after treatment with ND1-YL2 (20 μM) to CL1-YL2 (20 μM).

FIG. 17 is the results of Western blot analysis of SRC-1 and SRC-3 expression levels in MDA-MB-231 cells after treatment with ND1-YL2 (20 μM), YL2 (20 μM), MG132 (5 μM) to RLAA peptide (20 μM).

FIGS. 18A to 18F show that the cellular activity of ND1-YL2 is confirmed in MDA-MB-231 cells, which are breast cancer cells. Here, FIG. 18A shows an mRNA level of CSF-1 through real-time polymerase chain reaction; FIG. 18B shows an mRNA level of E-cadherin through real-time polymerase chain reaction; FIG. 18C is an image obtained by analyzing the degree of gap closure of MDA-MB-231 cells after treatment with YL2 (20 μM), RLAA peptide (20 μM) to ND1-YL2 (20 μM) for 72 hours; FIG. 18D is data quantifying the gap closure area as a percentage; FIG. 18E is an image of MDA-MB-231 cells invaded after 24 hours of treatment with YL2 (20 μM) to ND1-YL2 (20 μM), showing an analysis of the invasion ability of cells; and FIG. 18F is data quantified as a percentage.

FIG. 19 shows the results of a cytotoxicity test of ND1-YL2 on Colo205, MDA-MB-231, and HEK293T cells.

FIGS. 20A and 20B show a study on breast cancer cell metastasis of ND1-YL2 conducted through an in vivo mouse experiment. Here, FIG. 20A is a histogram of the flow cytometry of a single cell suspension in mouse lungs after injection of MDA-MB-231 cells treated with DMSO to ND1-YL2 into mice; and FIG. 20B is a representative image of metastatic tumor cells in mouse lungs, wherein a black arrow indicates metastatic tumor cells in the lungs.

FIG. 21 is a result of quantifying the flow cytometry histogram of FIG. 20A.

### BEST MODES OF THE INVENTION

Hereinafter, the present invention will be described in more detail.

As described above, since SRC-1 plays a very important role in cancer metastasis, degrading SRC-1 may suppress cancer metastasis. However, it is very difficult to develop a molecule that regulates the SRC-1 transcriptional activity because protein-protein interactions must be targeted. Several SRC-1 inhibitors have been developed so far, but there was a problem that they have very low activity or no selectivity for SRC-1.

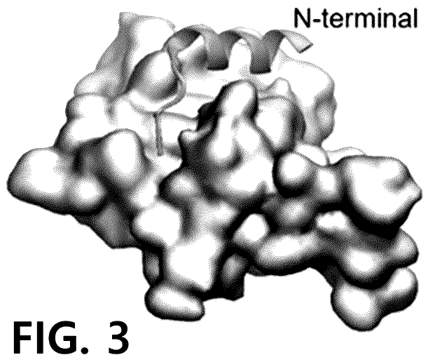

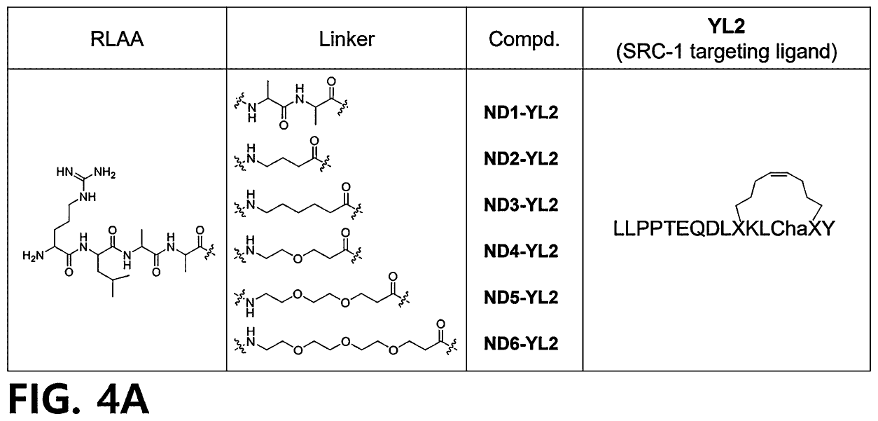

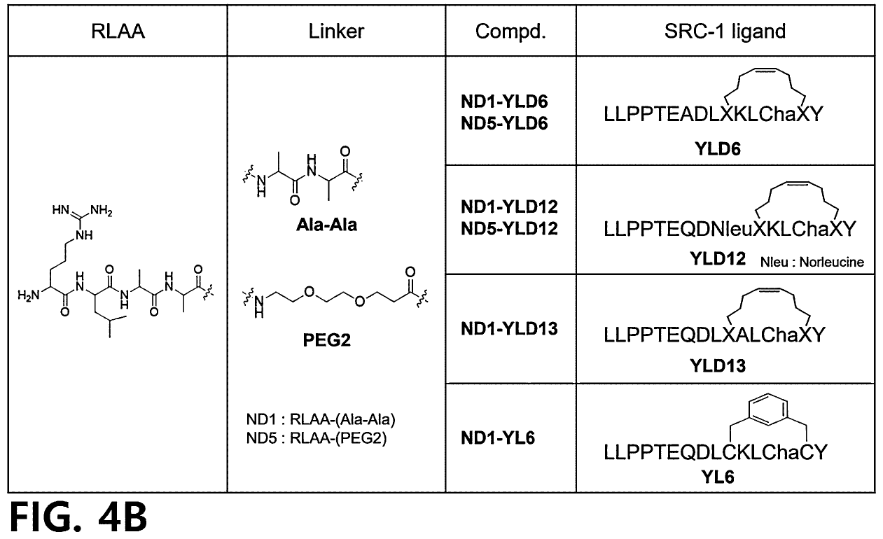

Meanwhile, the present inventors have developed a stapled peptide called YL2 that specifically binds to SRC-1 to be targeted (FIG. 2). Since the YL2 has a peptide sequence derived from the PAS-B domain of STAT-6, which is known to specifically bind to the SRC-1, it has a characteristic of being specific to the SRC-1 (FIG. 3).

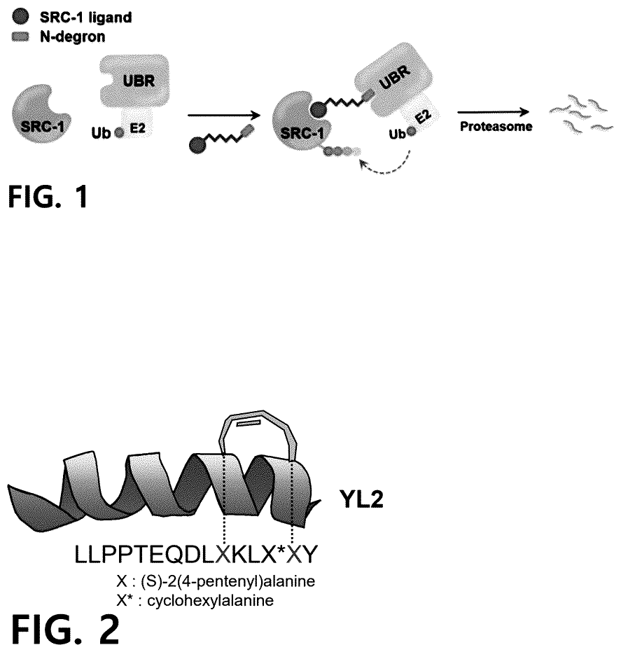

Accordingly, the present inventors attempted to solve the above problems by developing chimeric compounds in which YL2, a ligand that specifically binds to SRC-1, is linked to a ligand that binds to UBR protein (E3 ligase) (FIG. 1); and confirmed that the developed chimeric compounds actually bind effectively to SRC-1, ubiquitinate and degrade SRC-1, and thus have the effect of treating, preventing or suppressing metastasis of cancer caused by overexpression of SRC-1, thereby completing the present invention.

Accordingly, in one aspect, the present invention relates to a chimeric compound having a structure of Formula 1 below:

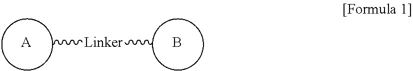

wherein:
A represents a ubiquitin ligase binding moiety (ULM) that binds to any one or more E3 ubiquitin ligases selected from the group consisting of ubiquitin-protein ligase E3 component n-recognin 1 (UBR1), ubiquitin-protein ligase E3 component n-recognin 2 (UBR2) and ubiquitin-protein ligase E3 component n-recognin 4 (UBR4), and
B represents a protein target moiety (PTM) that binds to steroid receptor coactivator-1 (SRC-1), wherein A and B are chemically linked by a linker.

The protein target moiety can bind to the steroid receptor coactivator-1 (SRC-1), and preferably may be a stapled peptide described in Korean Patent Publication No. 10-2019-0046260, which is incorporated herein by reference to the extent that it does not contradict the present invention.

The ubiquitin ligase binding moiety can bind to the E3 ubiquitin ligase, and may be any one or more selected from the group consisting of ubiquitin-protein ligase E3 component n-recognin 1 (UBR1), ubiquitin-protein ligase E3 component n-recognin 2 (UBR2) and ubiquitin-protein ligase E3 component n-recognin 4 (UBR4), preferably may be ubiquitin-protein ligase E3 component n-recognin 1 (UBR1).

When the chimeric compound binds simultaneously to protein and ubiquitin ligase, the protein may be ubiquitinated and degraded by the ubiquitin ligase.

The linker may have a structure of Formula 2:

—Y₁—Y₂—Y₃— [Formula 2]
wherein Y₁ is R 1, or Y₁ is absent;
R₁ is selected from the group consisting of —C(═O)N(H)—, —N(H)—, —N(H)C(═O)—, —O—, —CH₂—, —CH═CH— and —C—;
Y₂ is any one selected from the group consisting of —C(═O)N(H)—, —N(H)—, —N(H)C(═O)—, —O—, —CH₂—, —CH═CH— and —C≡C—; and
Y₃ is selected from the group consisting of —C(═O)—, —N(H)—, —C(═O)N(H)—, —N(H)C(═O)—, —O—, —CH₂—, —CH═CH— and —C≡C—, or may be absent.
Y₂ is selected from the group consisting of —CH₂ (CH₂ OCH₂)m1CH₂—, —(CH₂)m2—W—(CH₂)m3—, —(CH₂)m2—W—(CH₂)m4—O—(CH₂)m5— and —(N(H)CH(CH₃)C(═O))m6—;
W is selected from the group consisting of phenylene, five-membered heteroarene and cycloalkylene, or absent;
m1 is 1, 2, 3, 4, 5, 6 or 7;
m2 is 0, 1, 2, 3, 4, 5, 6 or 7;
m3 is 0, 1, 2, 3, 4, 5, 6 or 7;
m4 is 0, 1, 2, 3 or 4;
m5 is 0, 1, 2, 3 or 4; and
m6 is 0, 1, 2, 3 or 4.

In order to form a bond between the above ubiquitin ligase binding moiety (ULM) and the protein target moiety (PTM), these moieties may be altered in the structure of a portion thereof, and methods for such alteration are well known in the art.

The protein target moiety (PTM) comprises an amino acid sequence of X₁X₂X₃X₄X₅X₆X₇X₈X₉X₁₀X₁₁X₁₂X₁₃X₁₄X₁₅ (SEQ ID NO: 1),
wherein the amino acid sequence of SEQ ID NO: 1 is a stapled peptide in which two amino acids in the amino acid sequence of SEQ ID NO: 1 are linked to each other, and
in the amino acid sequence,
X₁, X₂, X₉ and X₁₂ may be valine (V), alanine (A), isoleucine (I), leucine (L), norleucine (Nleu), 3-methyl valine or norvaline;
X₃ and X₄ may be proline (P), hydroxy proline, amino proline, propynyl proline, chloro proline, bromo proline or trifluoromethyl proline;
X₅ may be threonine (T), serine (S), homoserine, methyl homoserine, or alanine (A);
X₆ may be glutamic acid (E), aspartic acid (D), or alanine (A);
X₇ may be glutamine (Q), asparagine (N), or alanine (A);
X₈ may be glutamic acid (E) or aspartic acid (D);
X₁₀ may be (S)-2-(4′-pentenyl) alanine, cysteine (C), homocysteine, lysine (K), ornithine (Orn) or diaminobutyric acid (Dab);
X₁₁ may be arginine (R), lysine (K) or alanine (A);
X₁₂ may be leucine (L) or alanine (A);
X₁₃ may be cyclohexylalanine (Cha), cyclopentylalanine (Cpa), cycloheptylpropanoic acid, phenylalanine (F), leucine (L), alanine (A), isoleucine (I) or valine (V);
X₁₄ may be (S)-2-(4′-pentenyl) alanine, cysteine (C), homocysteine, lysine (K), ornithine (Orn) or diaminobutyric acid (Dab); and
X₁₅ may be tyrosine (Y), serine (S), threonine (T) or alanine (A).

In the stapled peptide, amino acids at two positions selected from the group consisting of i, i+1, i+4, i+5, i+8, and i+9 in the amino acid sequence may be linked to each other, wherein i may be an integer.

The two amino acids may be linked through at least one bond selected from the group consisting of a carbon-carbon double bond, a carbon-carbon single bond, a carbon-nitrogen bond, and a carbon-sulfur bond.

When two amino acids selected from the group consisting of X₆, X₇, X₁₀, X₁₁, X₁₄ and X₁₅ in the amino acid sequence of SEQ ID NO: 1 are alanine, the two amino acids may be functionalized with a compound containing an alkenyl group.

The alkenyl group in the alkenyl group-containing compound may be at least one selected from the group consisting of a pentenyl group, a hexenyl group, a heptenyl group, an octenyl group, a nonenyl group, a decenyl group, an undecenyl group, and a dodecenyl group.

Two amino acids functionalized with the alkenyl group-containing compound can be linked by a ring produced through ring-closing metathesis, or can be so linked and then linked by a carbon-carbon single bond through a reduction reaction.

When two amino acids selected from the group consisting of X₆, X₇, X₁₀, X₁₁, X₁₄ and X₁₅ in the amino acid sequence of SEQ ID NO: 1 are cysteine, they may be linked by cyclization with a compound comprising a phenyl group.

The compound comprising a phenyl group may be represented by Formula 3 or Formula 4 below:

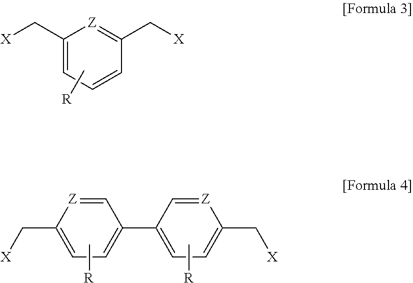

wherein X is at least one selected from the group consisting of chloro, bromo, and iodo; Z is nitrogen or oxygen; and R is at least one selected from the group consisting of hydrogen, halogen, C₁₋₄ alkyl, C₁₋₄ alkyl substituted with halogen, nitro, amino, and C₁₋₄ alkylamino.

When one of two amino acids selected from the group consisting of X₆, X₇, X₁₀, X₁₁, X₁₄ and X₁₅ in the amino acid sequence of SEQ ID NO: 1 are cysteine or homocysteine, and the other is lysine, ornithine or diaminobutyric acid, they may be linked by cyclization with a compound comprising triazine.

The stapled peptide of SEQ ID NO: 1 may have any one of the following sequences:

(SEQ ID NO: 2)
LLPPTEQDLTALChaLA,
(SEQ ID NO: 3)
LLPPTEQDLAKLChaAY,
(SEQ ID NO: 4)
LLPPTAQDLAKLChaLY,
(SEQ ID NO: 5)
LLPPTEADLTALChaLY,
(SEQ ID NO: 6)
LLPPTEQDLTCLChaLC,
(SEQ ID NO: 7)
LLPPTEQDLCKLChaCY,
(SEQ ID NO: 8)
LLPPTCQDLCKLChaLY,
(SEQ ID NO: 9)
LLPPTECDLTCLChaLY,
(SEQ ID NO: 10)
LLPPTEQDLX₁₆KLChaX₁₇Y,
(SEQ ID NO: 11)
LLPPTEQDLTX₁₈LChaLX₁₉,
(SEQ ID NO: 12)
LLPPTEADLAKLChaAY,
(SEQ ID NO: 13)
LLPPTEQDNleuAKLChaAY,
(SEQ ID NO: 14)
LLPPTEQDLAALChaAY
or
(SEQ ID NO: 15)
LLPPTEQDLCKLChaCY.

In each of the stapled peptides, two amino acids in an amino acid sequence within each stapled peptide may be linked to each other.

In SEQ ID NO: 10, X₁₆ is diaminobutyric acid (Dab) or cysteine (C), and X₁₇ is homocysteine, diaminobutyric acid (Dab), ornithine (Orn), or lysine (K);
in SEQ ID NO: 11, X₁₈ is cysteine (C), and X₁₉ is ornithine (Orn);
in SEQ ID NOs: 2 to 5, two alanines (A) in each amino acid sequence are functionalized with pentenyl and then are linked by a ring produced through ring-closing metathesis, or can be so linked and then linked by a carbon-carbon single bond through a reduction reaction;
in SEQ ID NOs: 6 to 9, cysteine (C) in each amino acid sequence is linked by cyclization with bromomethylphenyl; and
in SEQ ID NO: 10, diaminobutyric acid (Dab) of X₁₆ and homocysteine of X₁₇ are linked by cyclization with triazine, or cysteine (C) of X₁₆ and diaminobutyric acid (Dab), ornithine (Orn) or lysine (K) of X₁₇ may be linked by cyclization with cyanuric chloride.

In each of the above stapled peptides, Cha is cyclohexylalanine, and Nleu is norleucine.

The stapled peptide may be selected from the group consisting of:

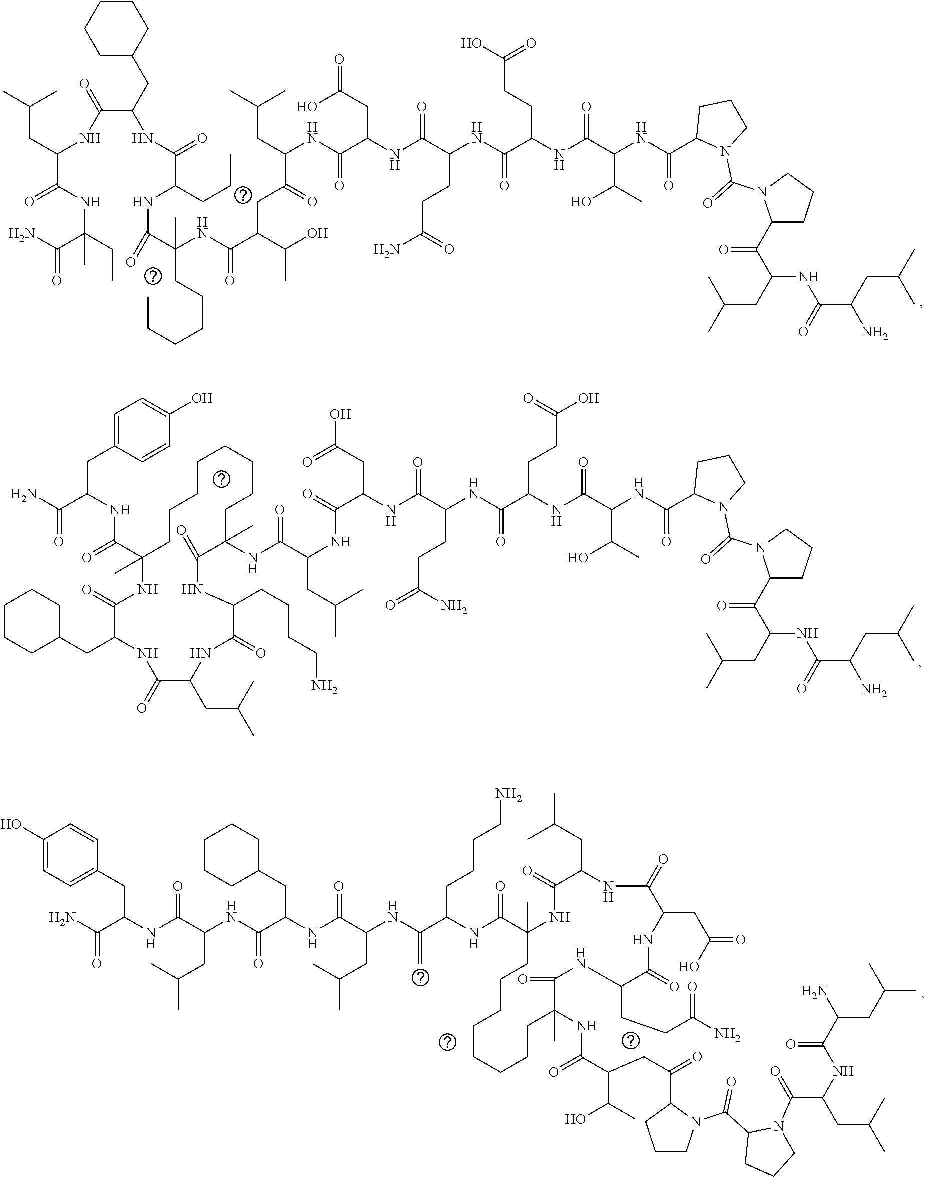

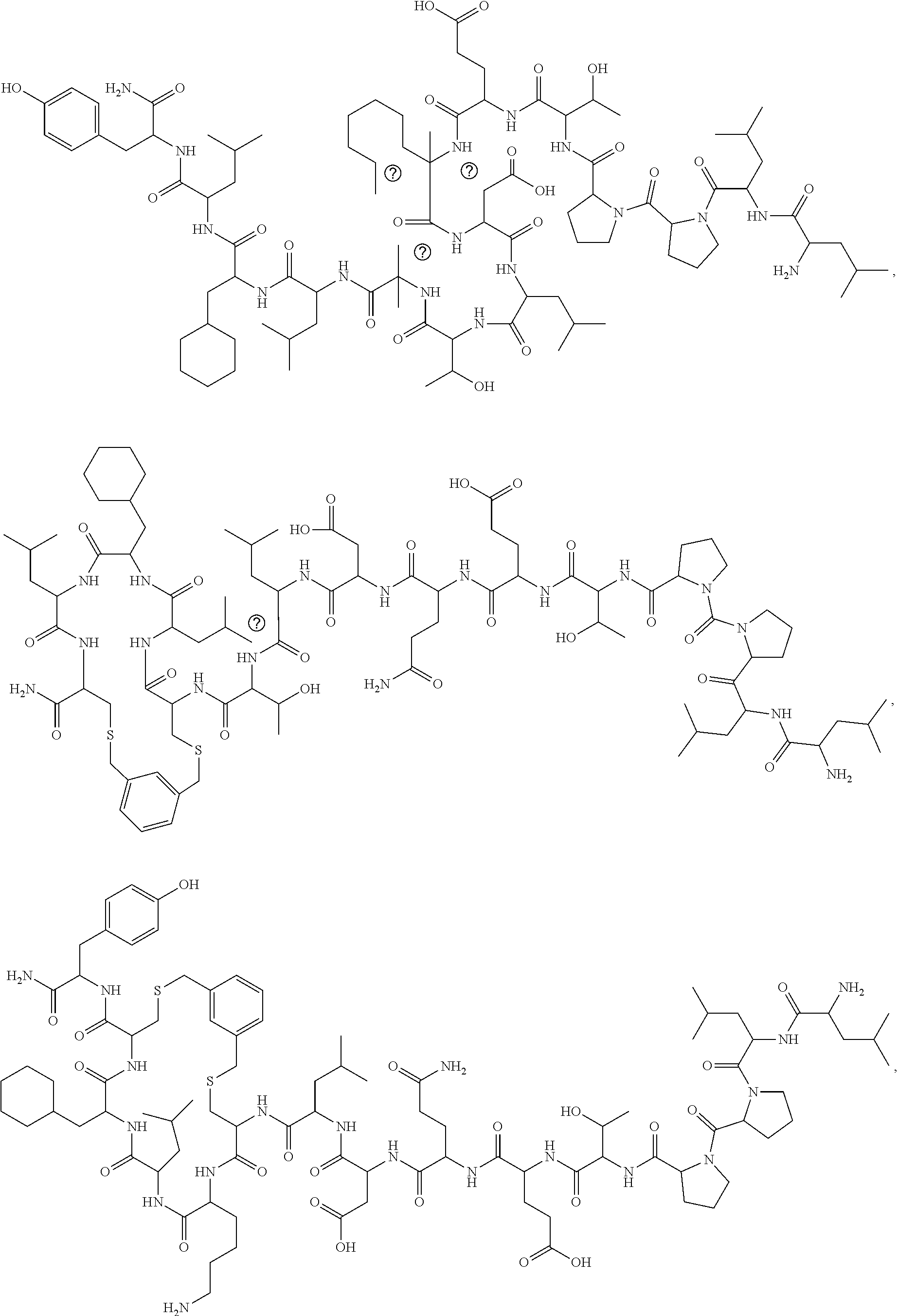

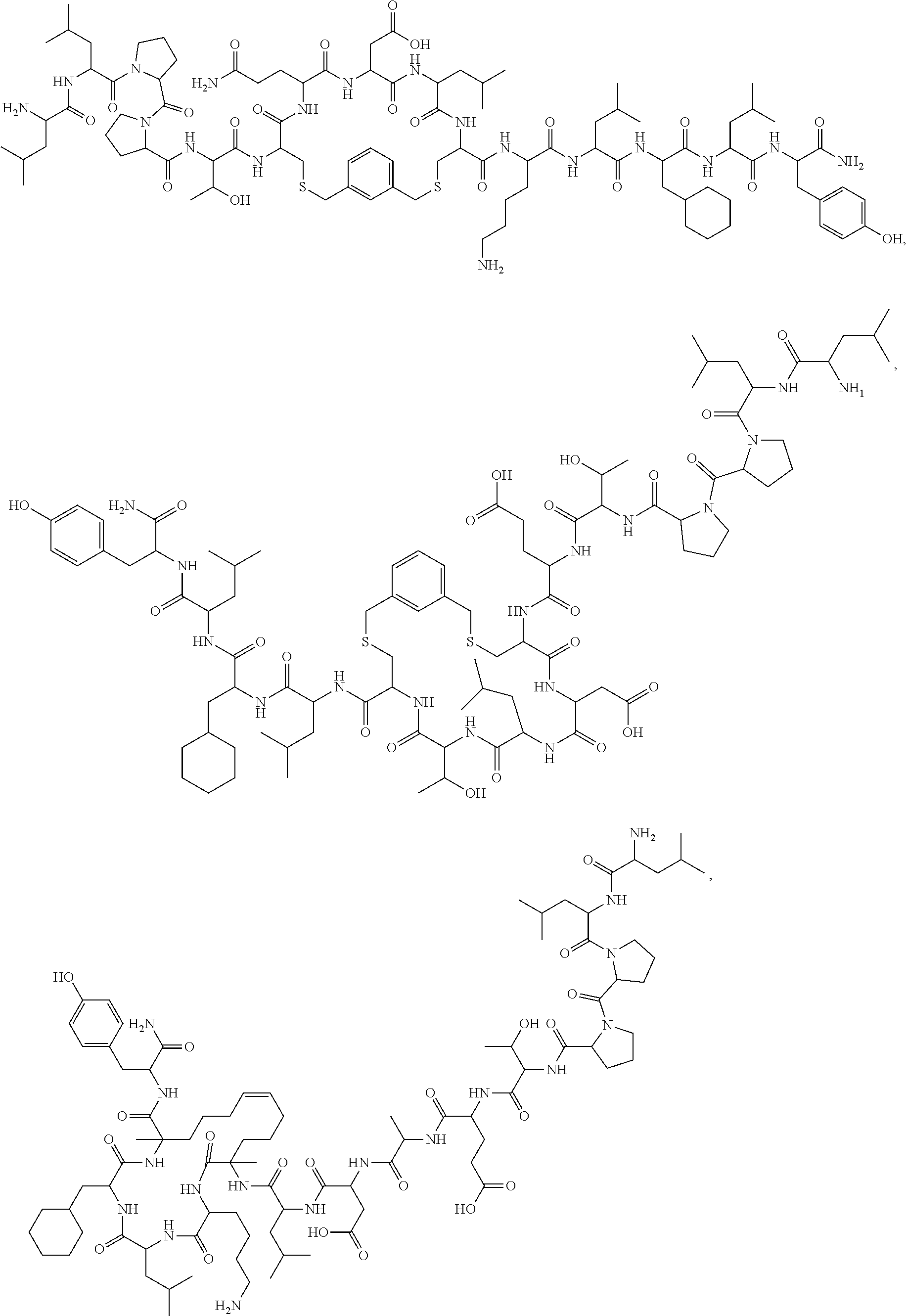

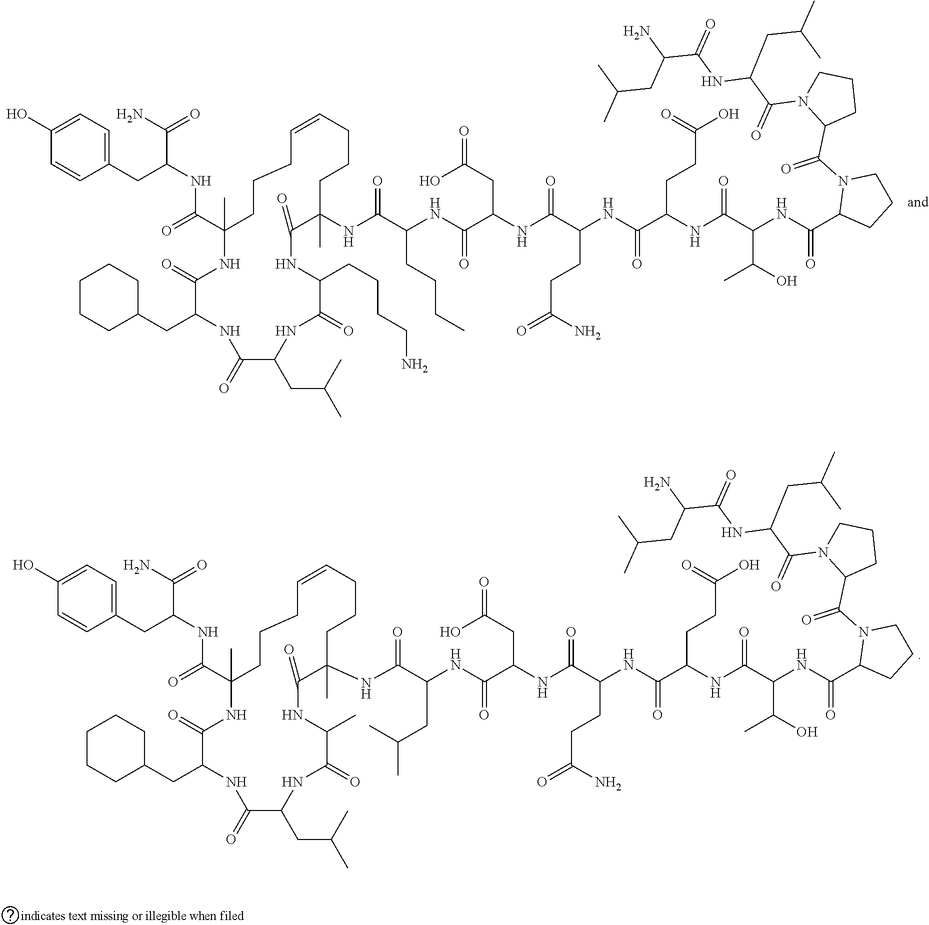 [SMILES: CCCCC(NC(=O)C(CC(=O)O)NC(=O)C(CCC(N)=O)NC(=O)C(CCC(=O)O)NC(=O)C(NC(=O)C1CCCN1C(=O)C1CCCN1C(=O)C(CC(C)C)NC(=O)C(N)CC(C)C)C(C)O)C(=O)NC1(C)CCC/C=C\CCCC(C)(C(=O)NC(Cc2ccc(O)cc2)C(N)=O)NC(=O)C(CC2CCCCC2)NC(=O)C(CC(C)C)NC(=O)C(CCCCN)NC1=O ; CC(C)CC(N)C(=O)NC(CC(C)C)C(=O)N1CCCC1C(=O)N1CCCC1C(=O)NC(C(=O)NC(CCC(=O)O)C(=O)NC(CCC(N)=O)C(=O)NC(CC(=O)O)C(=O)NC(CC(C)C)C(=O)NC1(C)CCC/C=C\CCCC(C)(C(=O)NC(Cc2ccc(O)cc2)C(N)=O)NC(=O)C(CC2CCCCC2)NC(=O)C(CC(C)C)NC(=O)C(C)NC1=O)C(C)O]

A linker may be coupled to the N-terminus or C-terminus of SEQ ID NO: 1.

The ubiquitin ligase binding moiety (ULM) may include an amino acid sequence of X₂₀X₂₁X₂₂X₂₃ (SEQ ID NO: 16):
X₂₀ may be arginine (R), histidine (H), lysine (K), phenylalanine (F), tyrosine (Y), isoleucine (I), tryptophan (W), glutamic acid (E) or aspartic acid (D);
X₂₁ may be arginine (R), leucine (L), isoleucine (I), alanine (A), valine (V), glycine (G) or phenylalanine (F), or absent;
X₂₂ and X₂₃ may be alanine (A), glycine (G) or valine (V), or absent.

In the present invention, the ubiquitin ligase binding moiety may include an amino acid sequence of RLAA (SEQ ID NO: 17).

The compound may include one or more selected from the group consisting of a plurality of ULMs, a plurality of PTMs, and a plurality of linkers.

Formula 1 may be a chimeric compound having any one formula selected from the group consisting of Formulas 5 to 16 below:

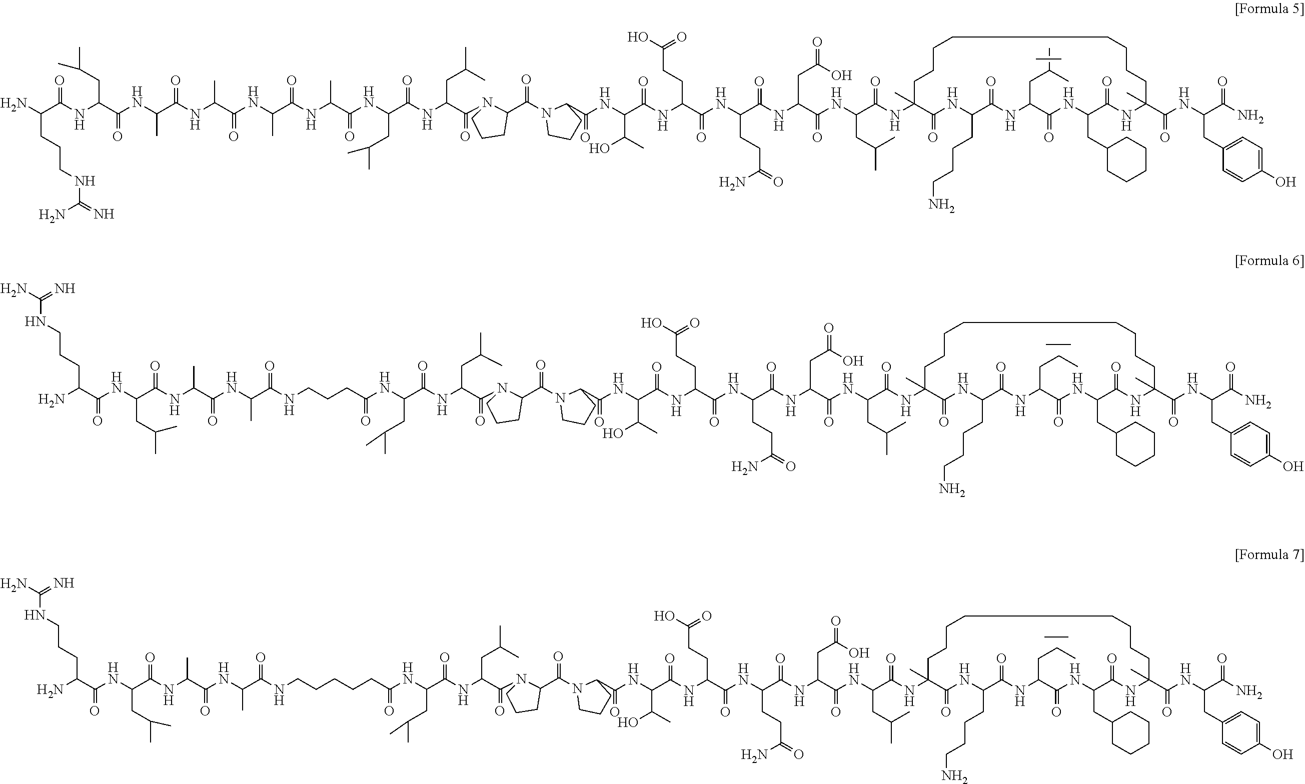

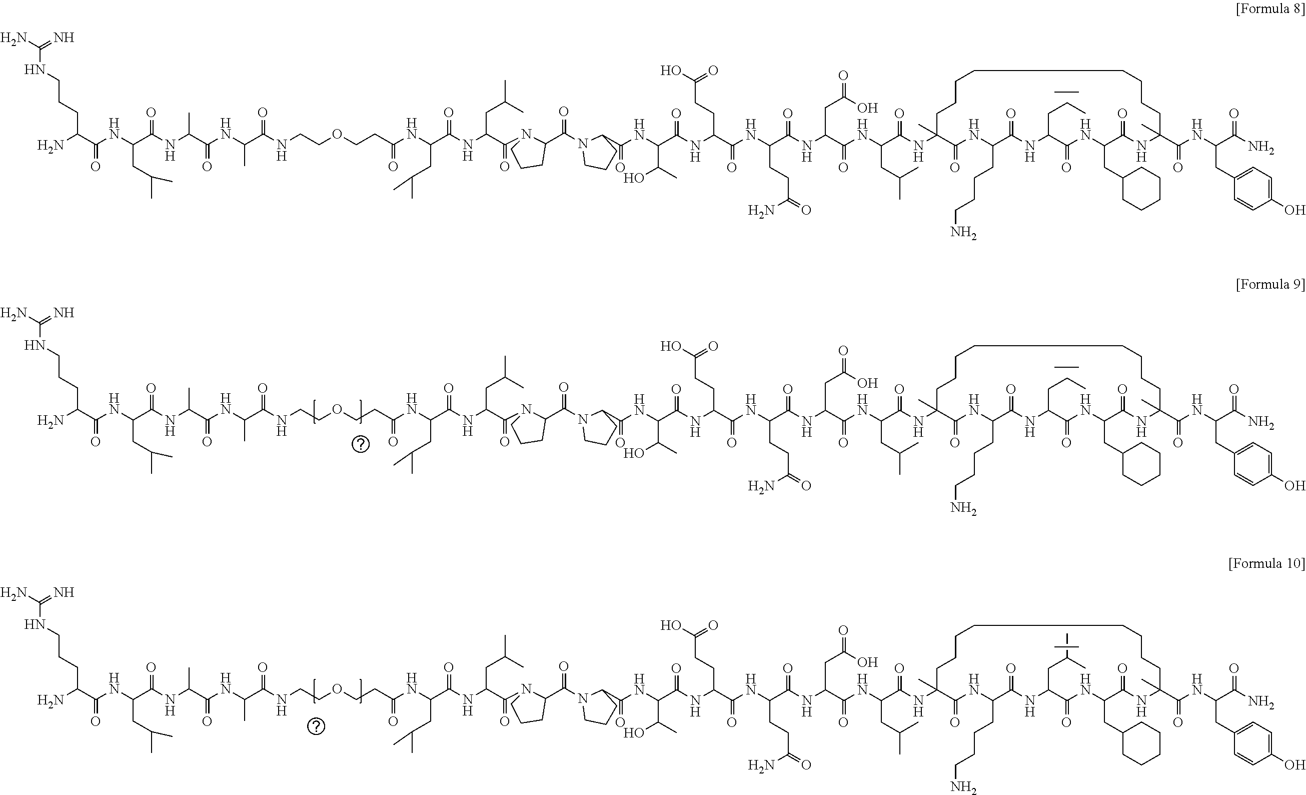

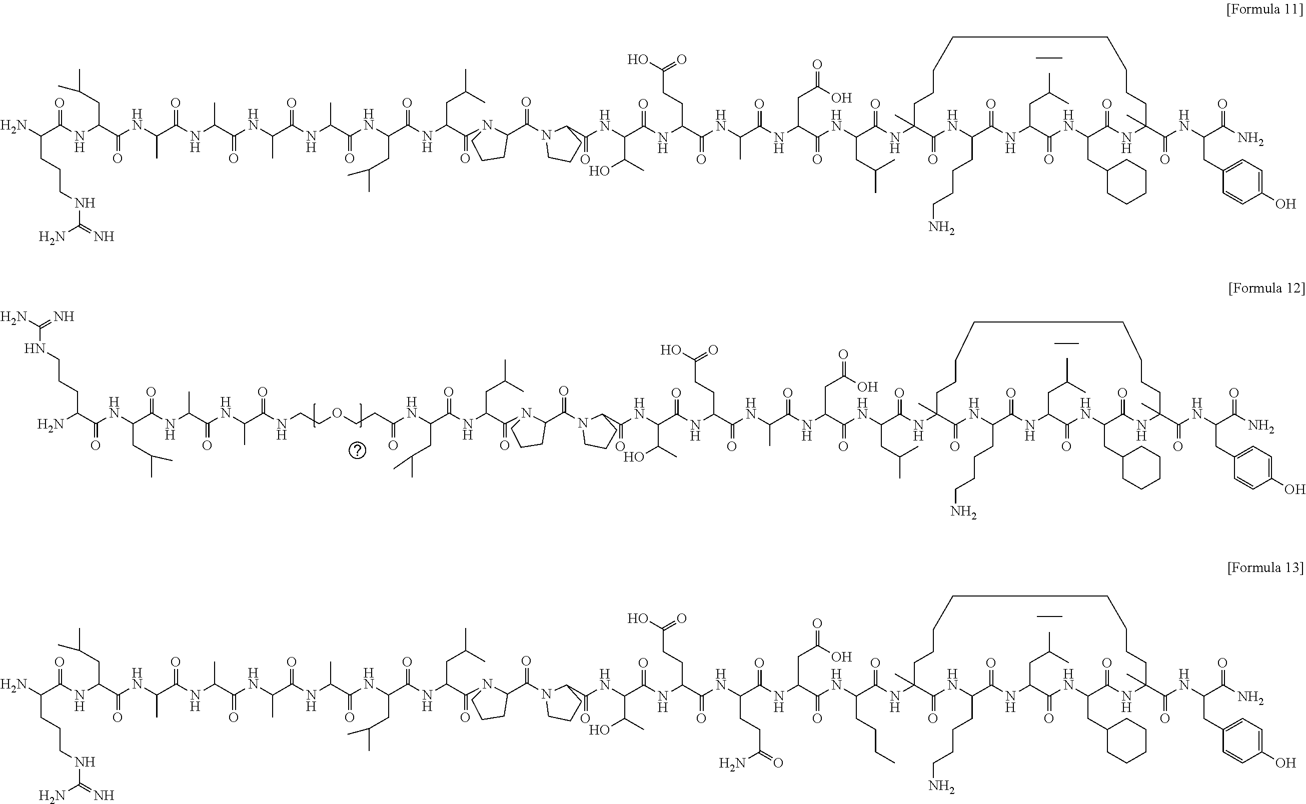

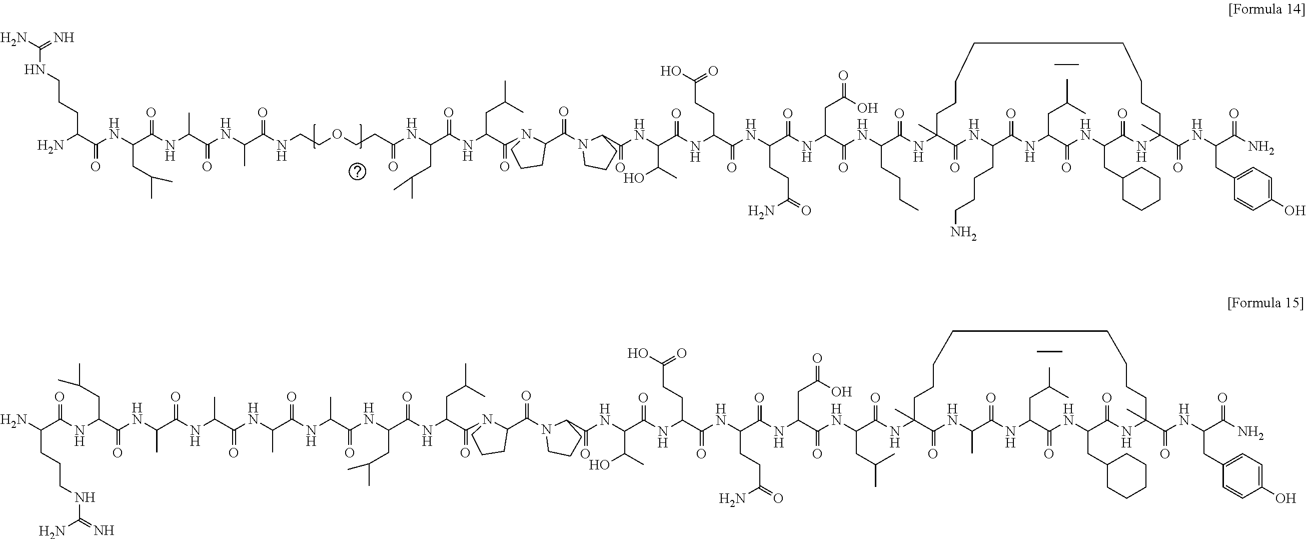

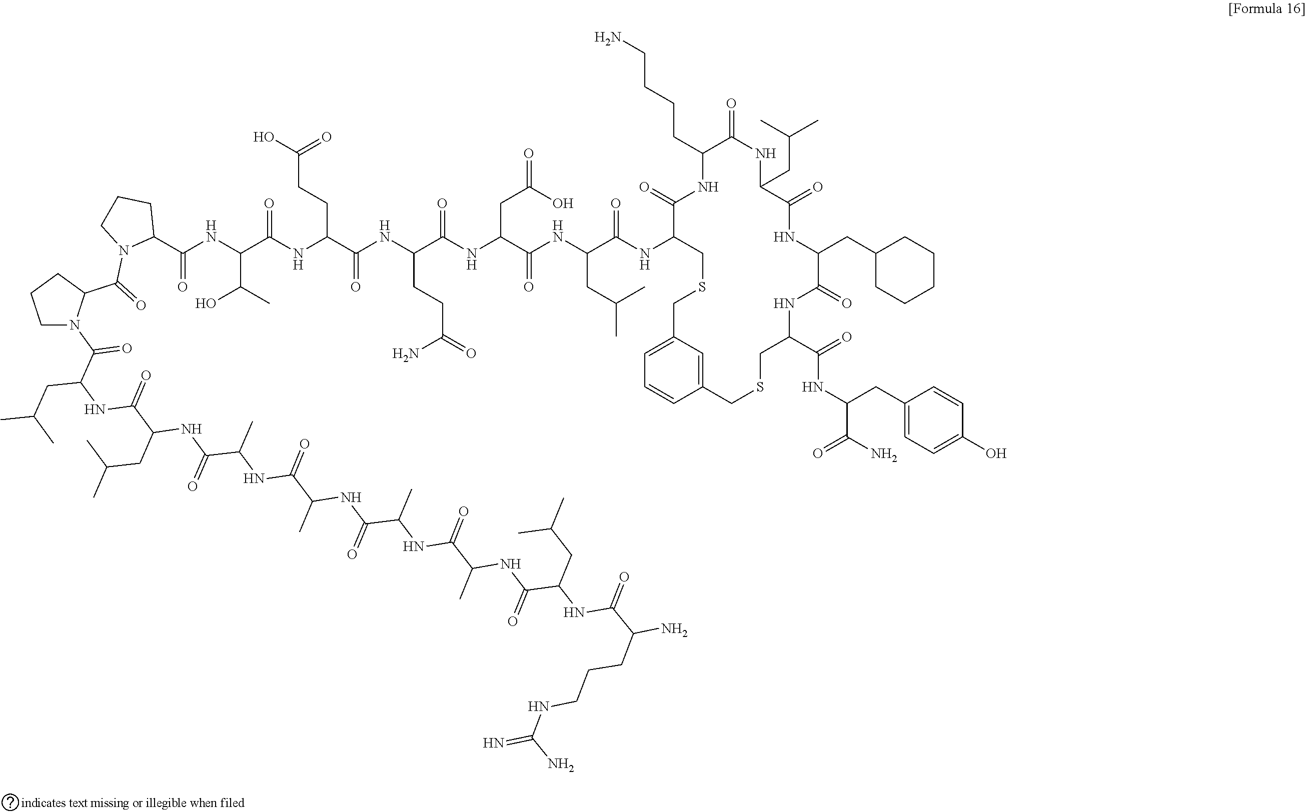 [SMILES: CC(C)CC(NC(=O)C(N)CCCNC(=N)N)C(=O)NC(C)C(=O)NC(C)C(=O)NC(C)C(=O)NC(C)C(=O)NC(CC(C)C)C(=O)NC(CC(C)C)C(=O)N1CCCC1C(=O)N1CCCC1C(=O)NC(C(=O)NC(CCC(=O)O)C(=O)NC(CCC(N)=O)C(=O)NC(CC(=O)O)C(=O)NC(CC(C)C)C(=O)NC1CSCc2cccc(c2)CSCC(C(=O)NC(Cc2ccc(O)cc2)C(N)=O)NC(=O)C(CC2CCCCC2)NC(=O)C(CC(C)C)NC(=O)C(CCCCN)NC1=O)C(C)O]

In the present invention, it was confirmed that the PROTAC chimera compound selectively degrades SRC-1 in cells and inhibits migration and invasion of cancer cells.

Accordingly, in another aspect, the present invention relates to a pharmaceutical composition for preventing or treating a disease caused by overexpression of SRC-1, such as an immune-related disease or cancer, comprising the various chimeric compounds described above, isomers, solvates or hydrates thereof.

In yet another aspect, the present invention relates to a use of any one of the chimeric compounds described above, isomers, solvates or hydrates thereof and/or the compositions for preventing or treating a disease caused by overexpression of SRC-1, such as an immune-related disease or cancer.

In yet another aspect, the present invention relates to a method for preventing or treating a disease caused by overexpression of SRC-1, such as an immune-related disease or cancer, the method comprising administering any one of the chimeric compounds described above, isomers, solvates or hydrates thereof and/or the compositions to a subject in need thereof.

In yet another aspect, the present invention relates to a use of any one of the chimeric compounds described above, isomers, solvates or hydrates thereof and/or the compositions for the preparation of a medicament for preventing or treating a disease caused by overexpression of SRC-1, such as an immune-related disease or cancer.

The immune-related disease may be any one or more selected from the group consisting of atopic dermatitis, asthma, airway hypersensitivity and chronic obstructive pulmonary disease, but is not limited thereto.

The cancer may be any one or more selected from the group consisting of breast cancer, prostate cancer, skin melanoma, thyroid cancer, and endometrial cancer, but is not limited thereto.

In yet another aspect, the present invention relates to a pharmaceutical composition for inhibiting metastasis of cancer caused by overexpression of SRC-1, comprising the various chimeric compounds described above, isomers, solvates or hydrates thereof.

In yet another aspect, the present invention relates to a use of any one of the chimeric compounds described above, isomers, solvates or hydrates thereof and/or the compositions for inhibiting metastasis of cancer caused by overexpression of SRC-1.

In yet another aspect, the present invention relates to a method of inhibiting metastasis of cancer, the method comprising administering any one of the chimeric compounds described above, isomers, solvates or hydrates thereof and/or the compositions to a subject in need thereof.

In yet another aspect, the present invention relates to a use of any one of the chimeric compounds described above, isomers, solvates or hydrates thereof and/or the compositions for the preparation of a medicament for inhibiting metastasis of cancer.

The cancer may be any one or more selected from the group consisting of breast cancer, prostate cancer, skin melanoma, thyroid cancer, and endometrial cancer.

In the present invention, the term “pharmaceutical composition” refers to a mixture comprising a chimeric compound of the present invention and a pharmaceutically acceptable excipient such as a diluent or a carrier. The pharmaceutical composition includes a composition for therapeutic use as well as a cosmetic composition. According to some embodiments, there is provided a method of administering a pharmaceutical composition comprising the compound of the present invention to a subject in need thereof. In some embodiments, the composition of the present invention may be administered to humans.

Although the description of the pharmaceutical compositions provided herein is principally concerned with pharmaceutical compositions intended for administration to humans, those skilled in the art will understand that such compositions are generally suitable for administration to all kinds of animals. A skilled veterinary pharmacologist has a good understanding of modifications in pharmaceutical compositions for administration to various animals, and, if necessary, can design and/or perform such modifications with mere routine experimentations.

The pharmaceutical compositions described herein may be prepared by any method known in the art of pharmacology or described hereinbelow. Generally, such manufacturing methods include associating an active ingredient with an excipient and/or one or more other auxiliary ingredients, followed by shaping and/or packaging the product into desired single- or multi-dose units, if necessary or desired.

The pharmaceutical composition of the present invention may be prepared, packaged and sold as a single unit dose and/or as a plurality of single unit doses, and/or may be sold without packaging. As used herein, the term “unit dose” refers to an individual amount of a pharmaceutical composition containing a predetermined amount of an active ingredient. The amount of active ingredients is generally equal to the dosage of active ingredients administered to a subject, and/or a convenient fraction of such dosage, for example, ½ or ⅓ of the dosage.

The relative amounts of active ingredients, pharmaceutically acceptable excipients, and/or any additional ingredients in the pharmaceutical compositions of the invention will vary depending on the identity, size and/or disorder of the subject to be treated and the route by which the composition is to be administered. For example, the composition may contain from 0.1% to 100% (w/w) of the active ingredient.

As used herein, pharmaceutically acceptable excipients include any and all of solvent, dispersion medium, diluent, or other liquid vehicle, dispersion or suspension aid, surface active agent, tonicity agent, thickener or emulsifier, preservative, solid binder, lubricant, etc. suitable for the purpose of a particular dosage form. Remington, The Science and Practice of Pharmacy, 21^{st} Edition, A. R. Gennaro, (Lippincott, Williams & Wilkins, Baltimore, MD, 2006) describes various excipients used in the formulation of pharmaceutical composition and techniques known for their preparation. A use of any conventional carrier medium is considered to be within the scope of the present invention, except that it is incompatible with a substance or its derivative, for example, by providing any unwanted biological effect or otherwise interacting with any other component of pharmaceutical compositions in a harmful manner. Pharmaceutically acceptable excipients are at least 95%, 96%, 97%, 98%, 99%, or 100% pure.

The excipients are approved for human and veterinary use. In some embodiments, the excipients are approved by the US Food and Drug Administration. In some embodiments, the excipients are pharmaceutical grade. In some embodiments, the excipients meet the standards of the United States Pharmacopoeia (USP), European Pharmacopeia (EP), British Pharmacopoeia, and/or International Pharmacopoeia (Ph. Int.).

Pharmaceutically acceptable excipients used in the preparation of pharmaceutical compositions include inert diluents, dispersants and/or granulizers, surface active agents and/or emulsifiers, disintegrants, binders, preservatives, buffers, lubricants, and/or oils, but are not limited thereto.

Such excipients may optionally be included in the formulations of the present invention. Excipients such as cocoa butter and suppository wax, colorants, coatings, sweeteners, flavors, and perfumes may be present in the composition at the discretion of the formulator.

Exemplary diluents include calcium carbonate, sodium carbonate, calcium phosphate, dicalcium phosphate, calcium sulfate, calcium hydrogen phosphate, sodium lactose phosphate, sucrose, cellulose, microcrystalline cellulose, kaolin, mannitol, sorbitol, inositol, sodium chloride, dried starch, corn starch, powdered sugar, and combinations thereof, but are not limited thereto.

Exemplary granulating and/or dispersing agents include potato starch, corn starch, tapioca starch, sodium starch glycolate, clay, alginic acid, guar gum, citrus pulp, agar, bentonite, cellulose and wood products, natural sponges, cation-exchange resins, calcium carbonate, silicate, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (veegum), sodium lauryl sulfate, quaternary ammonium compounds, and the combinations thereof, but are not limited thereto.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clay (e.g., bentonite [aluminum silicate] and veegum [magnesium aluminum silicate]), long-chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol); carbomers (e.g., carboxy polymethylene, polyacrylic acid, acrylic acid polymers, and carboxyvinyl polymers), carrageenan, cellulose derivatives (e.g., carboxymethyl cellulose sodium, powdered cellulose, hydroxymethyl cellulose, hydroxypropyl cellulose, hydroxypropyl methyl cellulose, methyl cellulose), sorbitan fatty acid esters (e.g., polyoxyethylene sorbitan monolaurate [Tween 20], polyoxyethylene sorbitan [Tween 60], polyoxyethylene sorbitan monooleate [Tween 80], sorbitan monopalmitate [span 40], sorbitan monostearate [span 60], sorbitan tristearate [span 65], glyceryl monooleate, sorbitan monooleate [span 80]), polyoxyethylene esters (e.g., polyoxyethylene monostearate [myrz 45], polyoxyethylene Hydrogenated castor oil, polyethoxylated castor oil, polyoxymethylene stearate, and solutol), sucrose fatty acid esters, polyethylene glycol fatty acid esters (e.g., cremophor), polyoxyethylene ethers (e.g., polyoxyethylene lauryl ether [Breeze 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, and/or combinations thereof, but are not limited thereto.

Exemplary binders include starches (e.g., corn starch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, shatty gum, mucilage of isapol husks, carboxymethyl cellulose, methyl cellulose, ethyl cellulose, hydroxyethyl cellulose, hydroxypropyl cellulose, hydroxypropyl methyl cellulose, microcrystalline cellulose, cellulose acetate, poly(vinyl-pyrrolidone), magnesium aluminum silicate (veegum), and larch arabinogalactan); alginate; polyethylene oxide; polyethylene glycol; inorganic calcium salts; silicic acid; polymethacrylate; wax; water; alcohol; and combinations thereof, but are not limited thereto.

Exemplary preservatives may include antioxidants, chelating agents, antimicrobial preservatives, antifungal preservatives, alcohol preservatives, acidic preservatives, and other preservatives. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite, but are not limited thereto. Exemplary chelating agents include ethylene diamine tetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal, but are not limited thereto. Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid, but are not limited thereto. Exemplary alcohol preservatives include ethanol, polyethylene glycol, phenol, phenolic compounds, bisphenol, chlorobutanol, hydroxybenzoate, and phenylethyl alcohol, but are not limited thereto. Exemplary acidic preservatives include vitamin A, vitamin C, vitamin E, beta-carotene, citric acid, acetic acid, dehydroacetic acid, ascorbic acid, sorbic acid, and phytic acid, but are not limited thereto. Other preservatives include tocopherol, tocopherol acetate, deteroxime mesylate, cetrimide, butylated hydroxyanisole (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glidant Plus, Fenonib, methylparaben, Germall 115, Germaben II, Neolone, Kathon, and Euxyl, but are not limited thereto. In certain embodiments, the preservative is an anti-oxidant. In another embodiment, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and combinations thereof, but are not limited thereto.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof, but are not limited thereto.

Exemplary oils include almond, apricot kernel, avocado, babassu palm, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, pumpkin, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oil, but are not limited thereto. Exemplary oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof, but are not limited thereto.

Liquid dosage forms for oral and parenteral administration include pharmaceutically acceptable emulsions, microemulsions, solutions, suspensions, syrups, and elixirs, but are not limited thereto. In addition to active ingredients, the liquid dosage forms may also include inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cotton seed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions may include adjuvants such as wetting agents, emulsifying and suspending agents, sweetening agents, flavoring agents, and perfuming agents. In certain embodiments for parenteral administration, the chimeric compounds of the present invention are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using dispersing agents, wetting agents and/or suspending agents. Sterile injectable preparations may be sterile injectable solutions, suspensions, or emulsions in nontoxic parenterally acceptable diluents or solvents such as 1,3-butanediol. Among the acceptable vehicles and solvents, water, Ringer's solution, U.S.P., and isotonic sodium chloride solution may be employed. Further, sterile, fixed oils are conventionally employed as a solvent or suspending medium. For this purpose, any bland fixed oil, including synthetic mono- or di-glycerides, may be employed. In addition, fatty acids such as oleic acid are used in the preparation of injectables.

Injectable formulations may be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This is accomplished by the use of a liquid suspension of crystalline or amorphous material with poor water solubility. The rate of absorption of the drug then depends upon the rate of dissolution which, in turn, may depend upon crystal size and crystalline form. Alternatively, delayed absorption of a parenterally administered drug is accomplished by dissolving or suspending the drug in an oil vehicle.

Solid dosage forms for oral administration include capsules, tablets, pills, powders, and granules. In such solid dosage forms, the active ingredient is mixed with one or more inert pharmaceutically acceptable excipients or carriers such as sodium citrate or dicalcium phosphate, and/or a) fillers or extenders such as starch, lactose, sucrose, glucose, mannitol, and silicic acid, b) binders such as, for example, carboxymethyl cellulose, alginates, gelatin, polyvinylpyrrolidinone, sucrose, and acacia, c) humectants such as glycerol, d) disintegrants such as agar, calcium carbonate, potato or tapioca starch, alginic acid, certain silicates, and sodium carbonate, e) dissolution retardants such as paraffin, f) absorption enhancers such as quaternary ammonium compounds, g) wetting agent such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

In the case of capsules, they may be formulated as tablets and pills, dosage forms may contain a buffering agent. Solid compositions of a similar type may also be employed as fillers in soft and hard-filled gelatin capsules using lactose or milk sugar as well as high molecular weight polyethylene glycols and the like as such excipients. Solid dosage forms such as tablets, sugar-coated tablets, capsules, pills, and granules can be prepared with coatings and shells, such as enteric coatings and other coatings well known in the pharmaceutical formulation art. They may optionally include opacifying agents, and may be of a composition that releases the active ingredient only, or preferentially, in a specific part of the intestinal tract, optionally in a delayed manner. Examples of embedding compositions which can be used include polymeric materials and waxes. Solid compositions of a similar type may also be employed as fillers in soft and hard-filled gelatin capsules using lactose or milk sugar as well as high molecular weight polyethylene glycols and the like as such excipients.

The active ingredient may be in a micro-encapsulated form with one or more excipients as described above. Solid dosage forms such as tablets, sugar-coated tablets, capsules, pills, and granules can be prepared with coatings and shells, such as enteric coatings, release controlling coatings, and other coatings well known in the pharmaceutical formulation art. In such solid dosage forms, the active ingredient may also be mixed with one or more inert diluents such as sucrose, lactose or starch. Such dosage forms may include additional substances other than inert diluents as is usually practiced, for example tablet lubricants and other tablet aids such as magnesium stearate and microcrystalline cellulose. In the case of capsules, they may be formulated as tablets and pills, dosage forms may also contain a buffering agent. They may optionally include opacifying agents, and may be of a composition that releases the active ingredient only, or preferentially, in a specific part of the intestinal tract, optionally in a delayed manner. Examples of embedding compositions which can be used include polymeric materials and waxes.

Dosage forms for topical and/or transdermal administration of a chimeric compound of the present invention may also include ointments, pastes, creams, lotions, gels, powders, solutions, sprays, inhalants and/or patches. Generally, the active ingredient is mixed, under sterile conditions, with a pharmaceutically acceptable carrier and/or any necessary preservatives and/or buffers that may be required. Additionally, the present invention contemplates the use of transdermal patches which often have additional advantages of providing controlled delivery of active ingredients to the body. Such dosage forms can be prepared, for example, by dissolving and/or dispersing the active ingredient in a suitable medium. Alternatively or additionally, the rate may be controlled by providing a rate controlling membrane and/or by dispersing the active ingredient in a polymer matrix and/or gel.

Formulations for topical administration include liquid and/or semi-liquid preparations such as liniments, lotions, oil in water and/or water in oil emulsions such as creams, ointments and/or pastes, and/or solutions and/or suspensions, but are not limited thereto. Although the concentration of the active ingredient may be as high as the solubility limit of the active ingredient in the solvent, topically-administrable formulations may include, for example, from about 1% to about 10% (w/w) of the active ingredient. Formulations for topical administration may further comprise one or more of the additional ingredients described herein.

The pharmaceutical composition of the present invention may be prepared, packaged and sold as a formulation for pulmonary administration via the oral cavity. Such formulations may also include dry particles comprising the active ingredient and having a diameter within a range of about 0.5 to about 7 nanometers or about 1 to about 6 nanometers. Such compositions are conveniently in the form of a dry powder for administration using a device comprising a dry powder reservoir into which a stream of propellant may be directed to disperse the powder, and for administration using a device comprising the active ingredient dissolved and/or suspended in a low-boiling propellant in a self-propelled solvent/powder dispensing vessel such as a sealed vessel. Such powders include particles in which at least 98% of the particles by weight have a diameter greater than 0.5 nanometers and at least 95% of the particles by number have a diameter less than 7 nanometers. Alternatively, at least 95% of the particles by weight have a diameter greater than 1 nanometer, and at least 90% of the particles by number have a diameter less than 6 nanometers. Dry powder compositions may also include a solid fine powder diluent such as a sugar and are conveniently provided in unit dose form.

The Low boiling propellant generally includes a liquid propellant having a boiling point below 65° F. at atmospheric pressure. In general, the propellant may constitute 50 to 99.9% (w/w) of the composition, and the active ingredient may constitute 0.1 to 20% (w/w) of the composition. The propellant may further comprise additional components such as a liquid non-ionic and/or solid anionic surfactant and/or a solid diluent (which may be of the same grade of particle size as the particles comprising the active ingredient).

The pharmaceutical compositions of the present invention formulated for pulmonary delivery may provide the active ingredient in droplet form of a solution and/or a suspension. Such formulations may be prepared, packaged, and/or sold as aqueous and/or dilute alcoholic solutions and/or suspensions, optionally sterilization containing the active ingredient, and may conveniently be administered using any nebulization and/or atomization device. Such formulations may further comprise one or more additional ingredients including, but not limited to, flavoring agents such as saccharin sodium, volatile oils, buffering agents, surface active agents, and/or preservatives such as methylhydroxybenzoate. Droplets provided by this route of administration may have an average diameter within a range of about 0.1 to about 200 nanometers.

Formulations described herein useful for pulmonary delivery are useful for intranasal delivery of the pharmaceutical compositions of the present invention. Another formulation for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nose.

Formulations for nasal administration may, for example, contain from about 0.1% (w/w) to 100% (w/w) of the active ingredient, and may also contain one or more additional ingredients described herein. The pharmaceutical composition of the present invention may be prepared, packaged and sold as a formulation for oral administration. Such formulations may be in the form of, for example, tablets and/or lozenges prepared in a conventional manner, and may include, for example, 0.1 to 20% (w/w) of the active ingredient, the balance comprising an orally dissolvable and/or degradable composition and, optionally, one or more of the additional ingredients described herein. Alternatively, formulations for oral administration may include powders and/or aerosolized and/or atomized solutions and/or suspensions containing the active ingredients. When dispersed, such powdered, aerosolized and/or atomized formulations may have a droplet size and/or average particle size within a range of about 0.1 to about 200 nanometers, and may further include one or more additional ingredients described herein.

The chimeric compounds of the present invention described herein are typically prepared in dosage unit form for easy and uniform administration. It will be appreciated, however, that the total daily usage of the compositions of the present invention may be determined by the attending physician within the scope of sound medical judgment. The specific therapeutically effective dose level for any particular subject depends on a variety of factors including disease, disorder, or disorder being treated and the severity of the disorder; the activity of the specific active ingredient employed; the specific composition employed; the subject's age, weight, general health, sex, and diet; the administration time, administration route, and excretion rate of the specific active ingredient employed; duration of treatment; drugs used in combination or coincidental with the specific active ingredient employed; and factors well known in the medical arts.

The chimeric compound of the present invention, a salt thereof, or a pharmaceutical composition thereof may be administered by any route. In some embodiments, the chimeric compound, salt thereof, or pharmaceutical composition thereof is administered by a variety of routes, including orally, intravenously, intramuscularly, intraarterially, intramedullarily, intrathecally, subcutaneously, intraventricularly, transdermally, intradermally, rectalally, intravaginally, intraperitoneally, topically (by powder, ointment, cream, and/or droplets), mucosal, nasal, buccal, enteral, sublingual; intratracheal instillation, bronchial instillation, and/or inhalation; and/or oral spray, nasal spray, and/or aerosol. Routes specifically contemplated are permeable intravenous injection, local administration via blood and/or lymphatic supply, and/or direct administration to the affected area. In general, the most suitable route of administration will depend on a variety of factors, including the characteristics of the agent (e.g., stability in the environment of the gastrointestinal tract), and the disorder of the subject (e.g., whether the subject can tolerate oral administration). Currently, oral and/or nasal spray and/or aerosol routes are most commonly used to deliver therapeutic agents directly to the lungs and/or respiratory system. However, the present invention includes delivery of the pharmaceutical composition according to the present invention by any suitable route taking into account advances in the field of drug delivery.

In certain embodiments, the chimeric compound, salt thereof, or pharmaceutical composition thereof of the present invention may be administered daily at a dosage level sufficient to deliver about 0.001 mg/kg to about 100 mg/kg, about 0.01 mg/kg to about 50 mg/kg, about 0.1 mg/kg to about 40 mg/kg, about 0.5 mg/kg to about 30 mg/kg, about 0.01 mg/kg to about 10 mg/kg, about 0.1 mg/kg to about 10 mg/kg, or about 1 mg/kg to about 25 mg/kg of body weight of a subject at least once a day to obtain the desired therapeutic effect. The desired dosage may be delivered three times a day, twice a day, every day, every two days, every three days, every week, every two weeks, every three weeks, or every four weeks. In certain embodiments, the desired dosage may be delivered via multiple administrations (e.g., 2, 3, 4, 5, 6, 7, 8, 9, 10, 11, 12, 13, 14 or more administrations).

It will be appreciated that the dose ranges described herein provide guidance for the administration of pharmaceutical compositions provided to an adult. For example, the amount to be administered to a child or adolescent can be determined by a physician or a person skilled in the art, and may be less than or equal to that administered to an adult. The exact amount of a peptide according to the present invention required to achieve an effective amount will vary from subject to subject, for example, depending on the subject's species, age, and overall disorder, severity of side effects or disorders, identity of the particular compound, mode of administration, and the like.

It will be appreciated that the chimeric compounds and pharmaceutical compositions of the present invention may be used in combination therapy. The particular combination of treatments (therapeutic agents or procedures) to be used in combination therapy will take into account the desired therapeutic effect to be achieved and the suitability of the desired therapeutic agent and/or procedure.

The pharmaceutical composition of the present invention may be administered alone or in combination with one or more therapeutically active agents. In the case of “combination”, although the following delivery method falls within the scope of the present invention, it is not intended to imply that the agents must be administered at the same time and/or formulated to be delivered together. The composition may be administered concurrently with, prior to, or after one or more other desired therapeutic agents or medical procedures. Generally, each agent will be administered at the dosage and/or time schedule defined for that agent. Additionally, the present invention encompasses delivery of the pharmaceutical compositions of the present invention in combination with an agent capable of improving the bioavailability, reducing and/or modifying the metabolism, inhibiting the secretion, and/or modifying the distribution in the body. It will be further appreciated that the chimeric compound of the present invention and the therapeutically active agent used in this combination can be administered together in a single composition or separately in different compositions.

The particular combination used in combination therapy will take into account the desired therapeutic effect to be achieved and/or the procedure comprising the peptides of the present invention and/or the suitability of therapeutically active agent. It will be appreciated that the combination used can achieve the desired effect for the same disorder (e.g, the chimeric compound of the invention may be administered in combination with another therapeutically active agent used to treat the same disorder), and/or can achieve different effects (e.g., control of any side effects).

As used herein, the term “therapeutically active agent” refers to any substance used as a medicament to treat, prevent, delay, reduce or ameliorate a disorder, and refers to a substance used in therapy, including prophylactic and curative treatments.

In some embodiments, the pharmaceutical composition of the present invention may be administered in combination with any therapeutically active agent or procedure (e.g., surgery, radiation therapy) which is useful for treating, mitigating, ameliorating, alleviating, delaying the onset of, inhibiting the progression of, reducing the severity of, and/or reducing the incidence of, one or more symptoms or features of cancer.

Hereinafter, the present invention will be described in detail by way of examples and the like in order to aid understanding of the present invention. However, the examples according to the present invention may be modified in various different forms, and the scope of the present invention should not be construed as being limited to the following examples. The examples of the present invention are provided to explain the present invention in more detail to those skilled in the art.

### EXAMPLES

### Example 1

### Synthesis of PROTAC Chimera Compound with N-Degron Bound to YL2

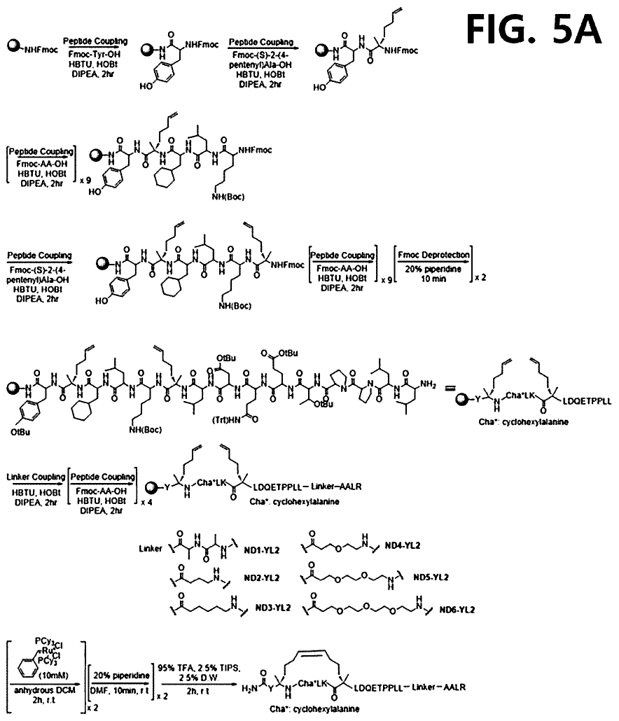

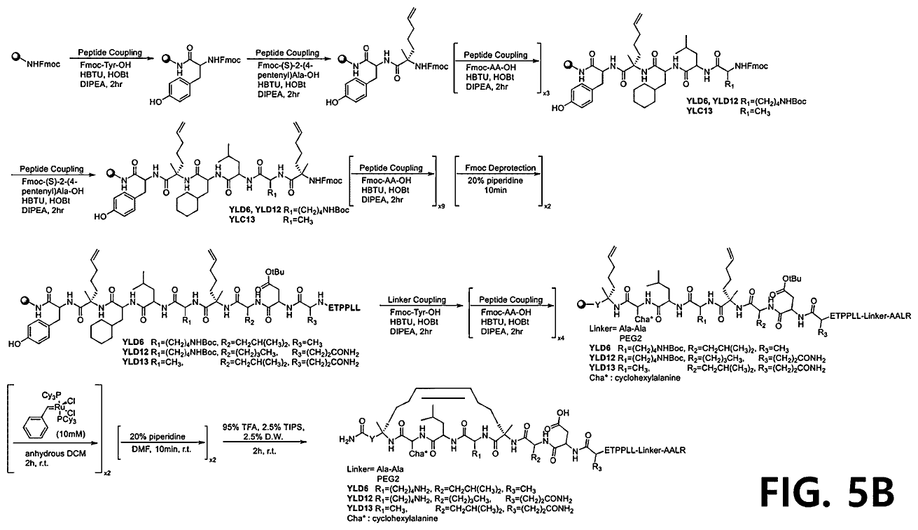

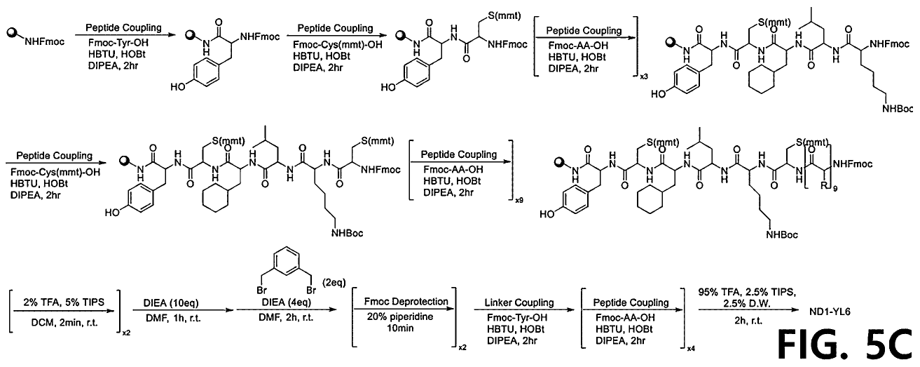

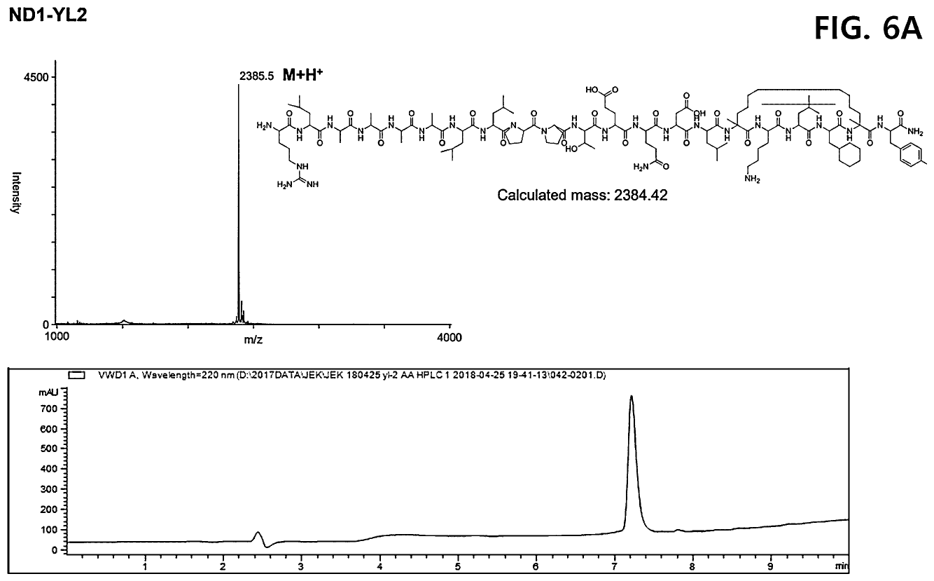

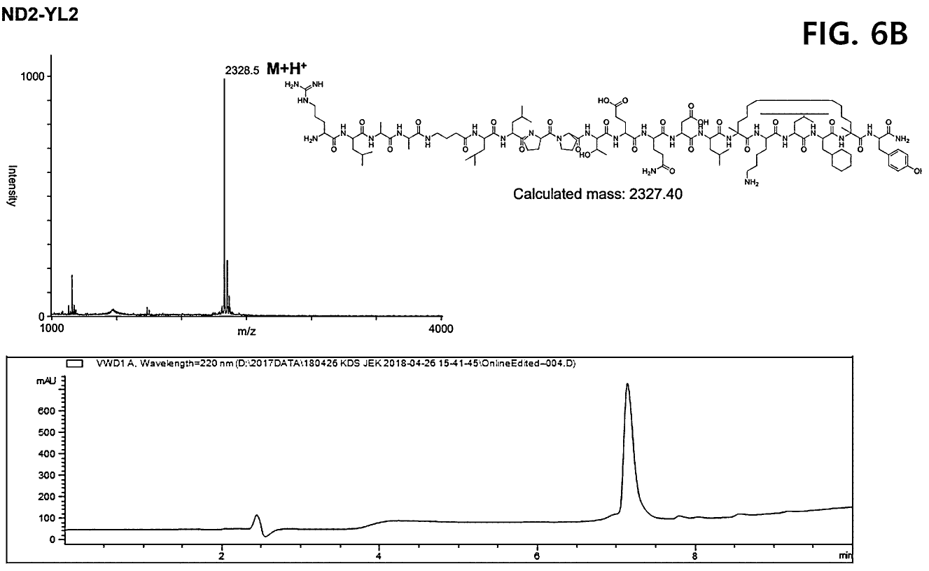

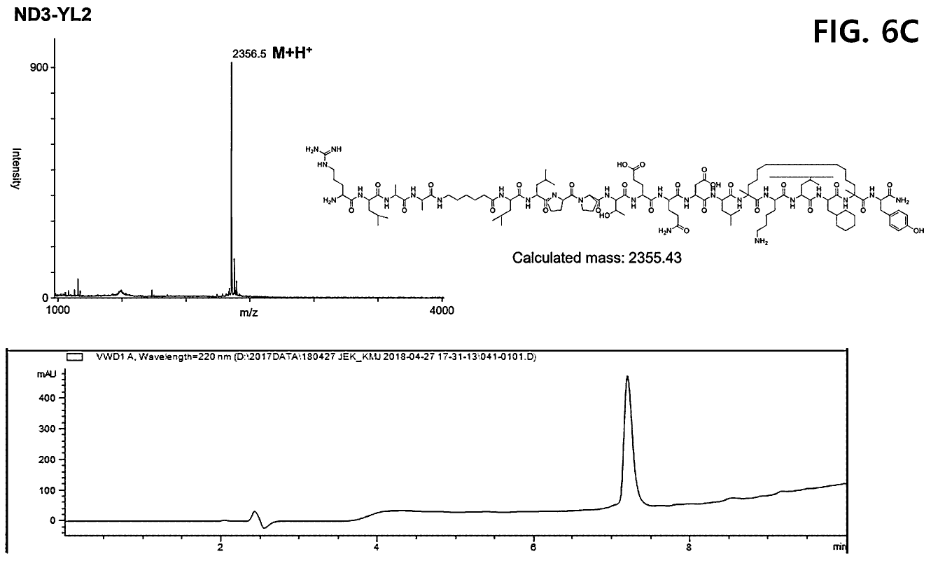

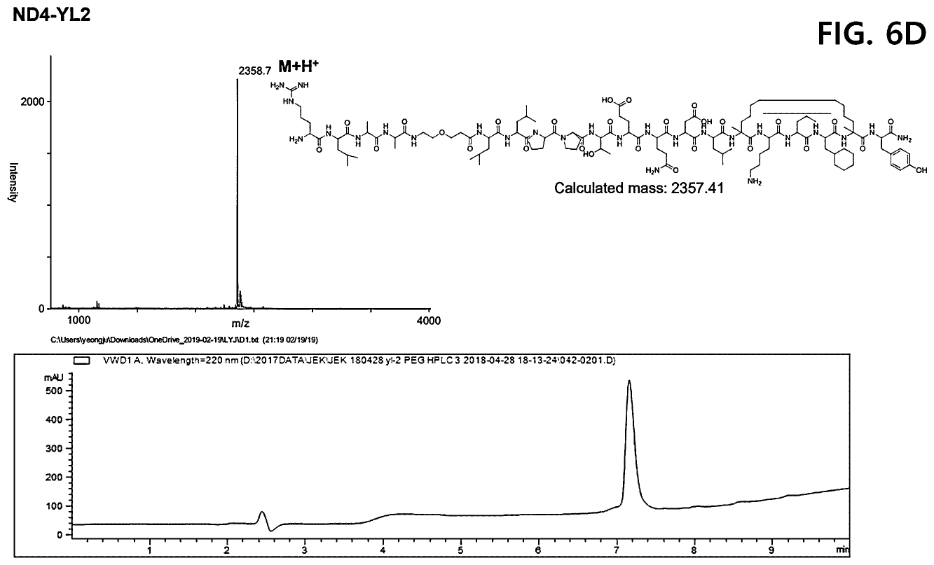

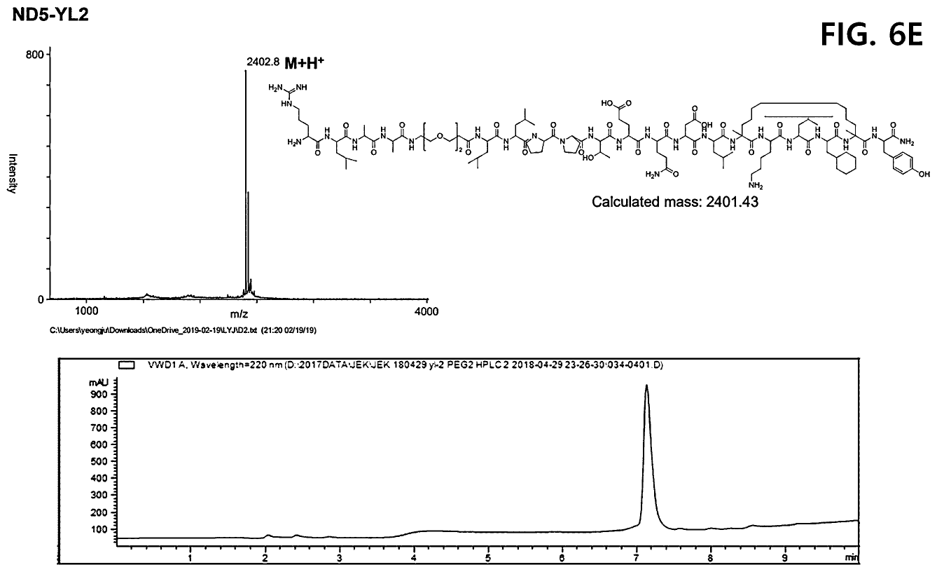

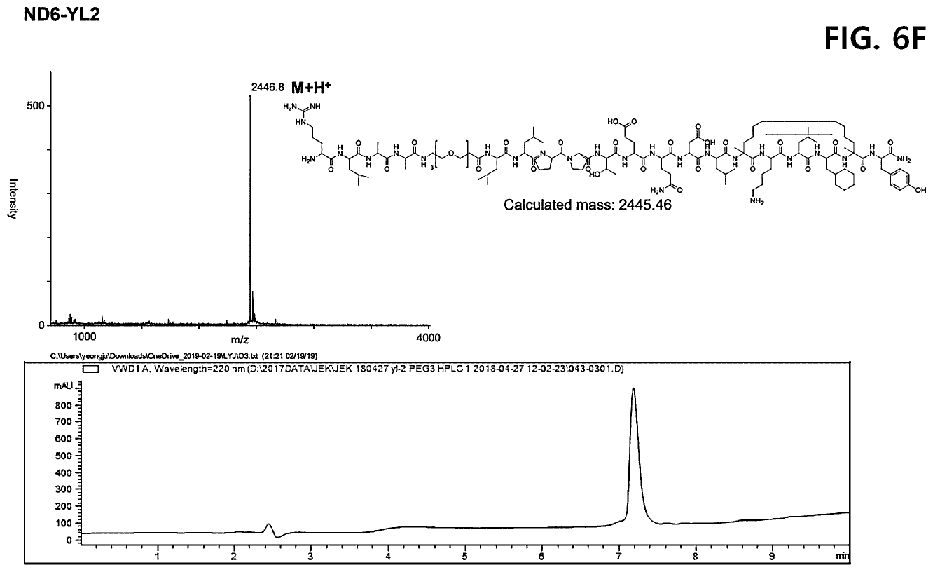

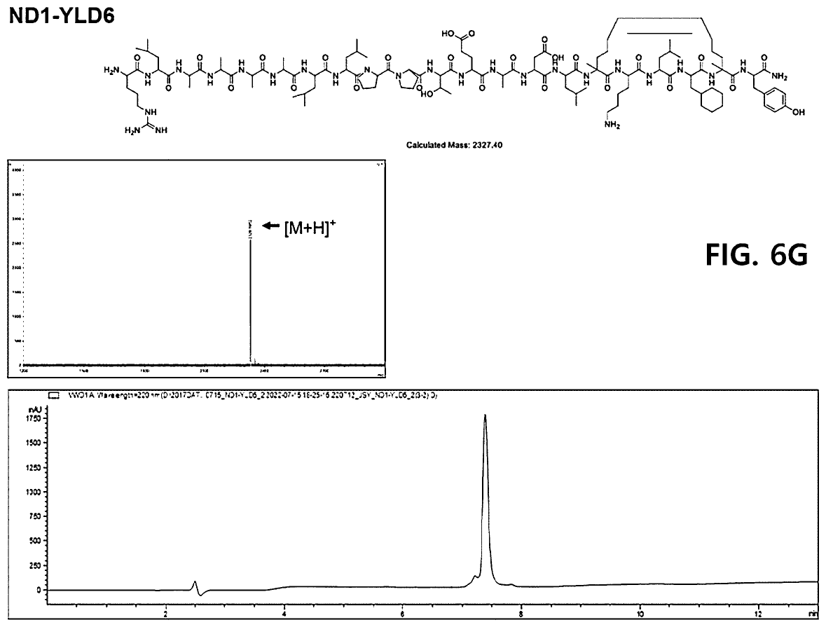

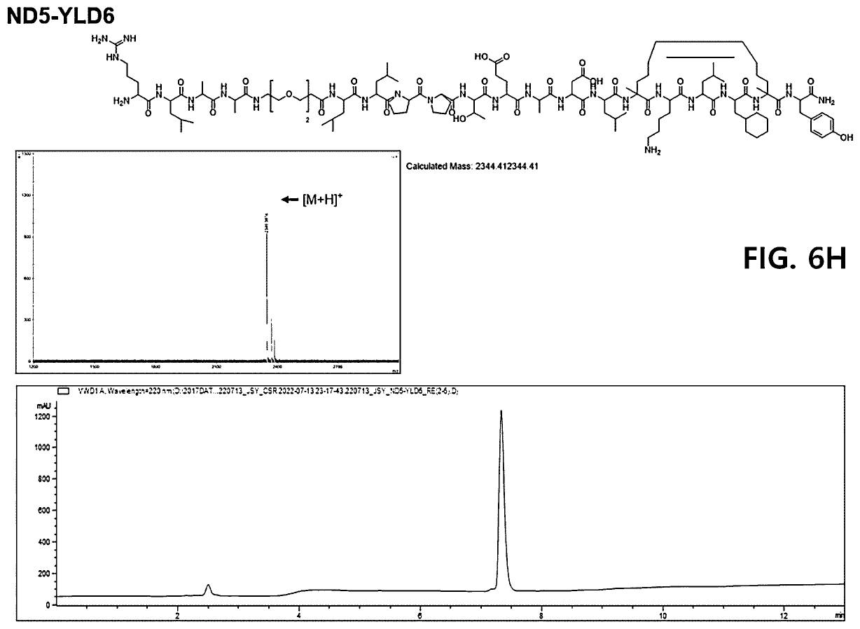

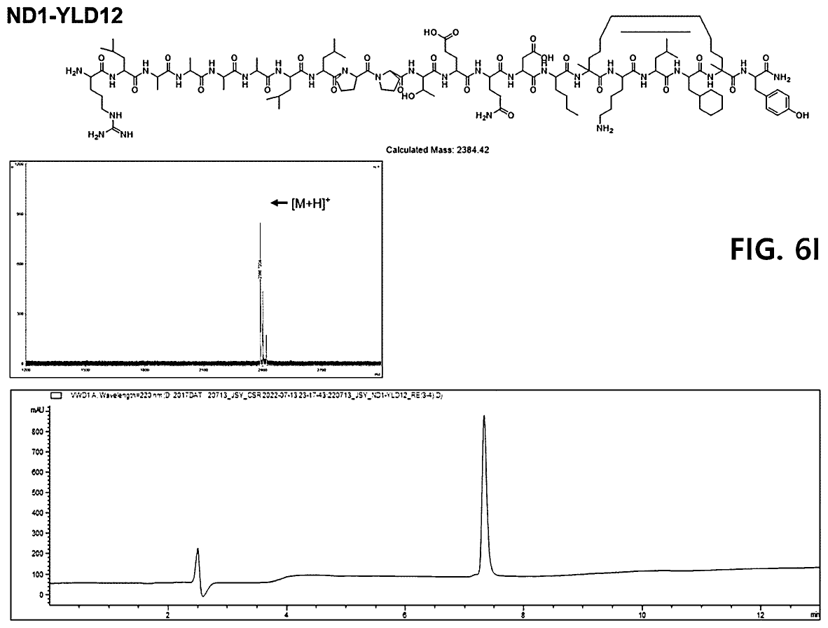

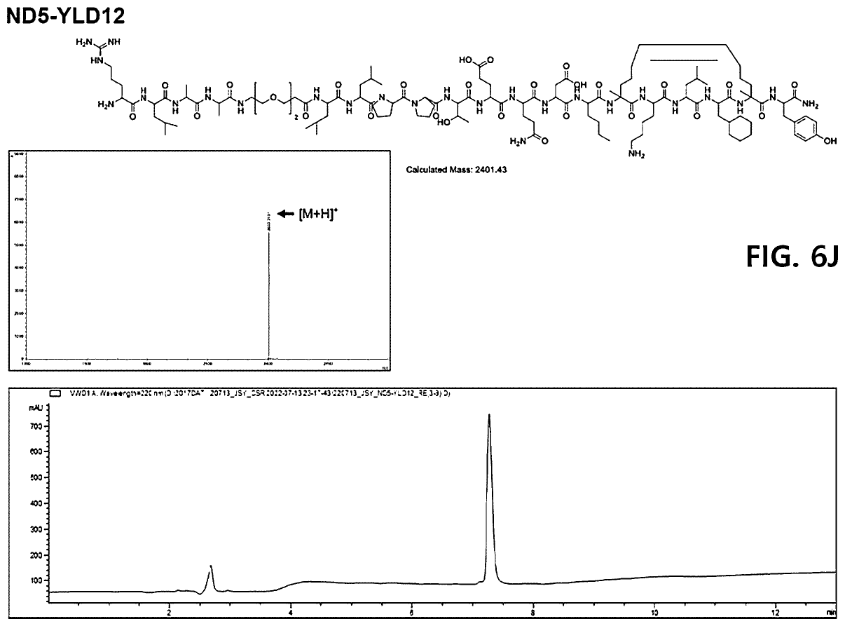

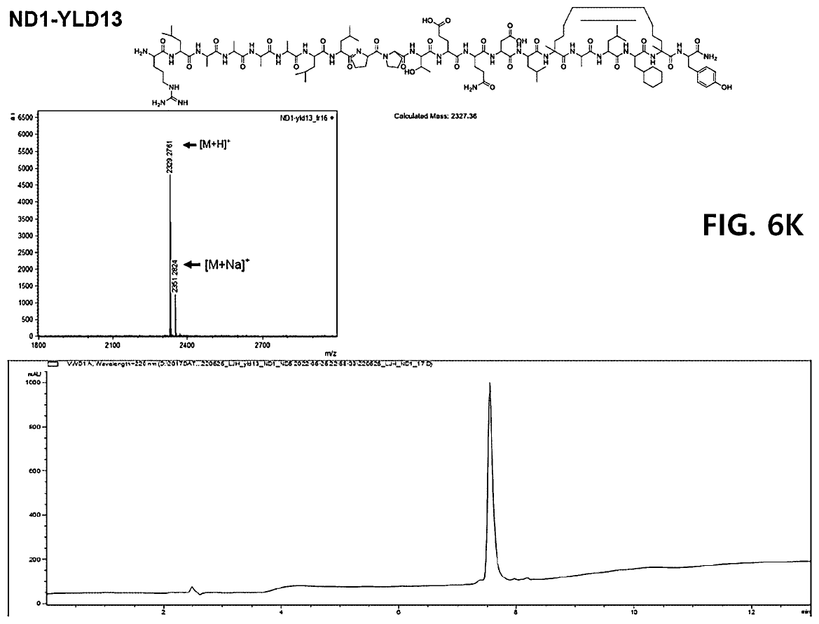

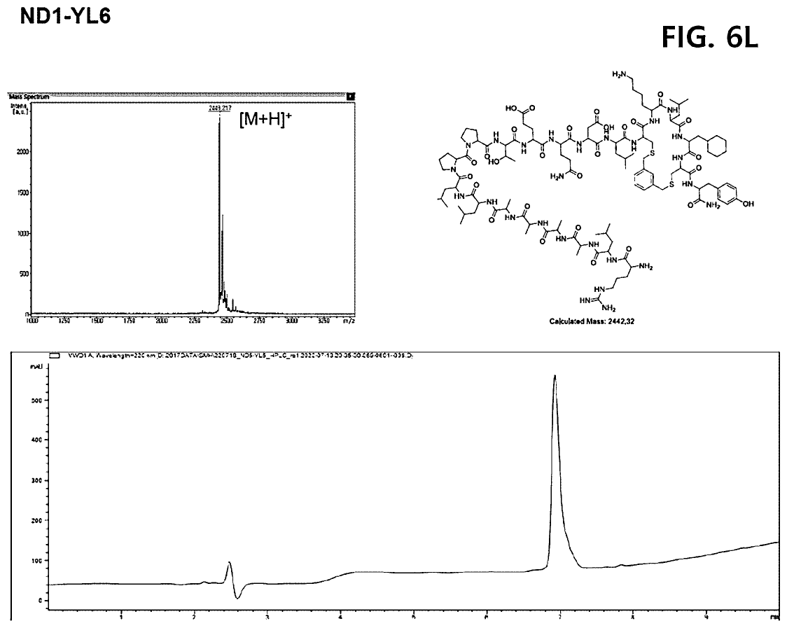

Rink amide MBHA resin (100 mg, 75 μmol) was put into a 5 mL frit syringe, and DMF was added thereto and left at room temperature for 2 hours. After removing the Fmoc protecting group with 20% piperidine in DMF (2×10 min), HBTU (5 equiv.), HOBt (5 equiv.), DIPEA (10 equiv.), and Fmoc-protected amino acid (5 equiv.) were treated to the beads. After the peptide coupling reaction at room temperature for 2 hours, the reaction mixture was discarded and the resin was washed with DMF (3×), MeOH (3×), CH₂Cl₂ (3×) and DMF (3×). This process was repeated to obtain the desired 20-residue or 21-residue peptides. The product was treated twice with 10 mM of a solution of bis(tricyclohexylphosphine)-benzylidene ruthenium(IV) dichloride (Grubbs' first generation catalyst) in CH₂Cl₂ at room temperature for 2 hours to perform an olefin metathesis reaction. After removal of the Fmoc protecting group, it was treated with 1 mL of cleavage cocktail (95% TFA, 2.5% TIS, and 2.5% DW) at room temperature for 2 hours to take the peptide off the resin and purify it by reverse phase HPLC (FIG. 5A, 5B, 5C). The MS and LC data of the purified ND1-YL2, ND2-YL2, ND3-YL2, ND4-YL2, ND5-YL2, ND6-YL2, ND1-YLD6, ND5-YLD6, ND1-YLD12, ND5-YLD12, ND1-YLD13, and ND1-YL6 are as shown in FIGS. 6A to 6L.

### Example 2

### Cell Culture

MDA-MB-231 cells were cultured at 37° C. in Dulbecco's modified Eagle's medium (DMEM) supplemented with penicillin-streptomycin containing 10% fetal bovine serum (FBS) and 5% CO₂. Colo205 and A549 cells were cultured at 37° C. in Roswell Park Memorial Institute medium supplemented with penicillin-streptomycin with 10% FBS and 5% CO₂.

### Example 3

### Confirmation of Target Protein Degradation of PROTAC Chimeric Compounds

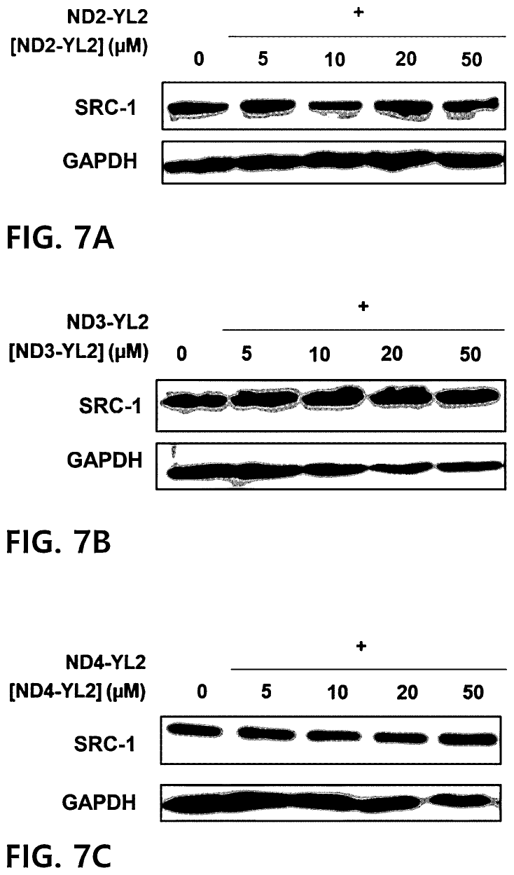

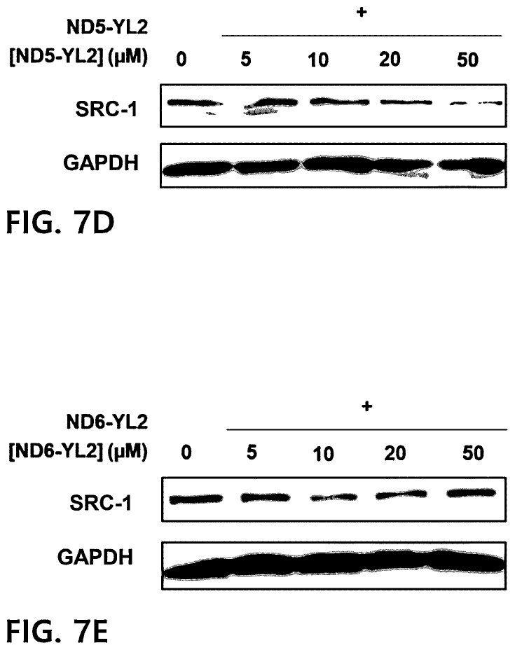

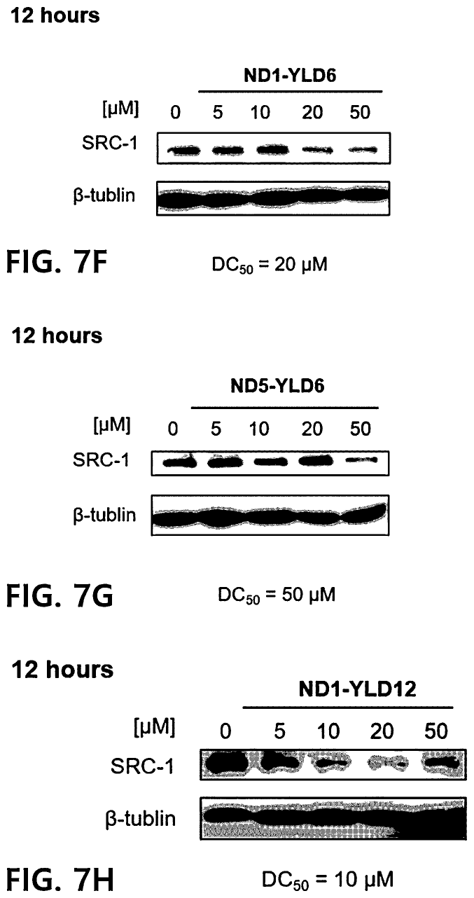

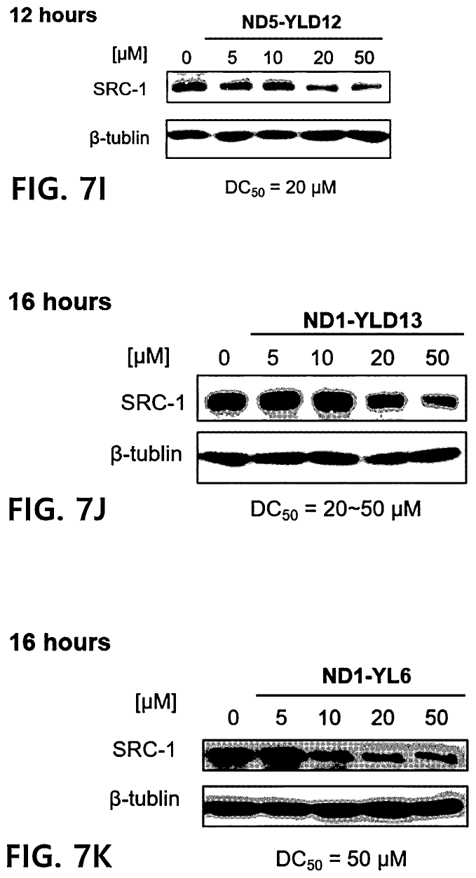

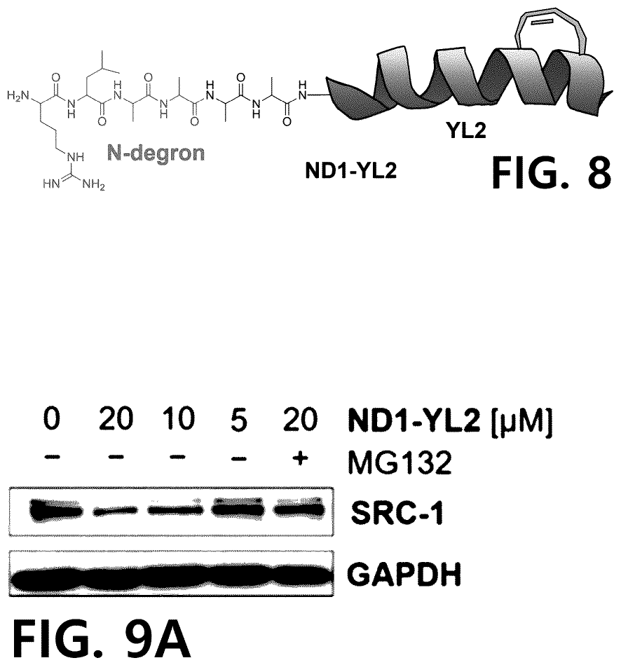

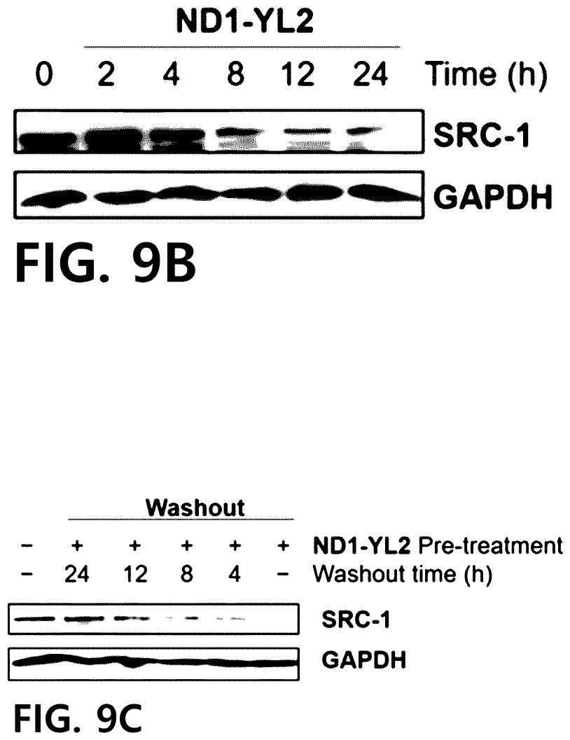

To evaluate the ability of the PROTAC chimeric compound synthesized in Example 1 to degrade SRC-1 in cells, human triple negative breast cancer (TNBC) MDA-MB-231 cells were treated with DMSO or various concentrations of the synthesized PROTAC chimeric compound. MDA-MB-231 cells were seeded at 5×10⁵ cells per well in a 6-well plate(Corning). After incubation at 37° C. for 24 hours, the cells were treated with the synthesized compounds in Opti-MEM medium. The cells were washed twice with cold Dulbecco's phosphate buffered saline (DPBS) prior to cell lysis. In order to lyse the cells, the cells on ice were treated with lysis buffer (50 mM TrisHCl pH 7.4, 150 mM NaCl, 1% TritonX, 1 mM EDTA, 1 mM DTT and 1×protease inhibitor cocktail). The cell lysate was centrifuged at 13,000×rpm for 15 minutes at 4° C. The supernatant was collected and the protein concentration was determined by Pierce™ 660 nm protein assay. 6×SDS loading buffer was added to the cell lysate, which was heated at 95° . C. for 5 minutes. The same amount of protein was loaded on SDS-PAGE, which was transferred to a PVDF membrane. The PVDF membrane was blocked with 5% skim milk in TBST (tris buffered saline containing 0.01% Tween-20) and treated with a primary antibodys at 4° C. for 12 hours. After incubation of the HRP-conjugated secondary antibody at room temperature for 1 hour, Western blot images were obtained with ECL solution. It was observed that SRC-1 was degraded by treatment with the PROTAC chimeric compound (FIGS. 7A to 7K). Among the tested compounds, ND1-YL2 having two alanines as a linker reduced an expression level of SRC-1 in a dose-dependent manner with a DC 50 of ˜10 μM, and showed the strongest activity among the synthesized and treated compounds (FIGS. 8 and 9A).

### Example 4

### Confirmation of SRC-1 Degradation by the Proteasome-Mediated Pathway and the N-Degron Pathway

To verify whether ND1-YL2 induces SRC-1 degradation through a proteasome-mediated pathway, SRC-1 was treated with ND1-YL2 and MG-132, a well-known proteasome inhibitor, or treated alone with ND1-YL2. MDA-MB-231 cells were seeded at 5×10⁵ cells per well in a 6-well plate (Corning). After incubation at 37° C. for 24 hours, the cells were treated with the synthesized compounds in Opti-MEM medium. The cells were washed twice with cold Dulbecco's phosphate buffered saline (DPBS) prior to cell lysis. In order to lyse the cells, the cells on ice were treated with lysis buffer (50 mM Tris·HCl pH 7.4, 150 mM NaCl, 1% TritonX, 1 mM EDTA, 1 mM DTT and 1×protease inhibitor cocktail). The cell lysate was centrifuged at 13,000×rpm for 15 minutes at 4° C. The supernatant was collected and the protein concentration was determined by Pierce™ 660 nm protein assay. 6×SDS loading buffer was added to the cell lysate, which was heated at 95° C. for 5 minutes. The same amount of protein was loaded on SDS-PAGE, which was transferred to a PVDF membrane. The PVDF membrane was blocked with 5% skim milk in TBST (tris buffered saline containing 0.01% Tween-20) and treated with a primary antibodys at 4° C. for 12 hours. After incubation of the HRP-conjugated secondary antibody at room temperature for 1 hour, Western blot images were obtained with ECL solution. The degradation of SRC-1 by ND1-YL2 did not show the effect of degrading SRC-1 when treated together with MG-132, a well-known proteasome inhibitor, whereby it has been proven that the degradation of SRC-1 was through a proteasome-mediated pathway. In addition, in order to evaluate the degradation of SRC-1 according to the treatment time of ND1-YL2, MDA-MB-231 cells were treated with ND1-YL2 (20 μM) and then cultured, and the cell level of SRC-1 was measured by Western blot over time. As shown in FIG. 9B, the degradation of SRC-1 was evident at 8 hours after treatment with ND1-YL2, and was maintained for 24 hours without detectable recovery of SRC-1 level. Then, after washing ND1-YL2, the expression level of SRC-1 was confirmed over time. As a result, it was confirmed that the expression level of SRC-1 was restored to the original level within 12 hours after ND1-YL2 was removed (FIG. 9C). This indicates that the degradation of SRC-1 by ND1-YL2 is reversible, unlike the method of regulating protein expression using the conventional genetic methods.

### Example 5

### Circular Dichroism (CD) Measurement

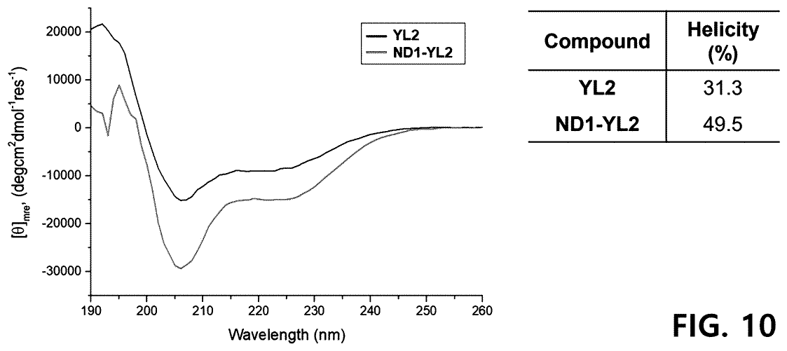

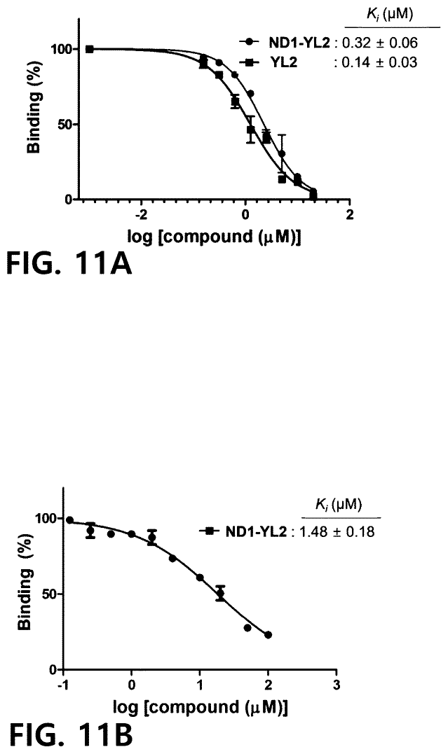

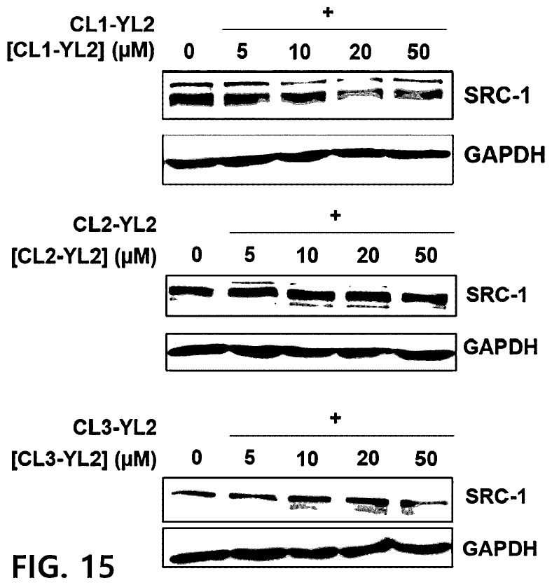

To achieve effective degradation of SRC-1, it is essential that the chimeric peptide ND1-YL2 binds to SRC-1 and UBR proteins simultaneously, resulting in the formation of a ternary complex. In order to confirm this, first, it was attempted to determine whether ND1-YL2 maintains an alpha helix structure which has an important effect on binding to the surface of SRC-1. To this end, a circular dichroism (CD) spectrum was measured. The CD spectrum was obtained from a Jasco J-815 spectropolarimeter (FIG. 15). The lyophilized peptide was dissolved in a solution (pH 7.4) of 70% acetonitrile and 30% sodium phosphate to a final concentration of 50 μM peptide. The CD spectrum was obtained using a quartz cuvette with a 2 mm path length. The spectrum was the average of five consecutive accumulations using a 100 nm/min scan rate. The raw data were converted into molar ellipticity per residue (deg·cm²·dmol⁻¹·residue⁻¹) as calculated per mole of amide group present and normalized by the molar concentration of peptide. Then, a smoothing and correction of the background spectrum was performed. This data was fitted using Origin Pro 9.0. The α-helical orientation of YL2 and ND1-YL2 was calculated by the α-helical orientation formula [Biochemistry 1974, 13, 3350-3359]. The ND1-YL2 was found to exhibit a CD spectrum similar to that of YL2, a compound previously reported to bind to SRC-1, indicating that the conjugation between the linker and the RLAA tetrapeptide did not affect the alpha helix structure of the original stapled peptide YL2 (FIG. 10).

### Example 6

### Protein Expression and Purification

An expression plasmid of the PAS-B domain of human SRC-1 (residues 257-385) tagged with His6 was provided by Professor John. A. Robinson. (ChemBioChem 2008, 9, 1318-1322). The plasmid was transformed into BL21 (DE3) pLysS cells, and the protein was cultured in Luria-Bertani broth medium and induced with 0.5 mM IPTG at 18° C. for 16 hours. Cell disruption was performed by ultrasonic treatment in a buffer (50 mM Tris-HCl pH 8.0, 200 mM NaCl and 1 mM TCEP), and insoluble materials were removed by centrifugation. HisTrap™ column (GE Healthcare, 17-5255-01) chromatography was performed on the buffer and 500 mM of imidazole was applied for elution. In order to remove the His₆-tag, TEV protease was used and the cleaved samples were loaded in HisTrap™ as described above. The SRC-1 was further purified by size exclusion chromatography (HiLoad™ 16/600 Superdex 75 pg) in a final buffer of 20 mM Tris-HCl pH 7.5, 150 mM NaCl and 1 mM TCEP. The expression and purification of the UBR box (residues 113-194) in the UBR1 E3 ubiquitin ligase of S. cerevisiae was performed in the previous manner. (Nat. Struct. Mol. Biol. 2010, 17, 1175-1181).

### Example 7

### Competitive Fluorescence Polarization (FP) Assay

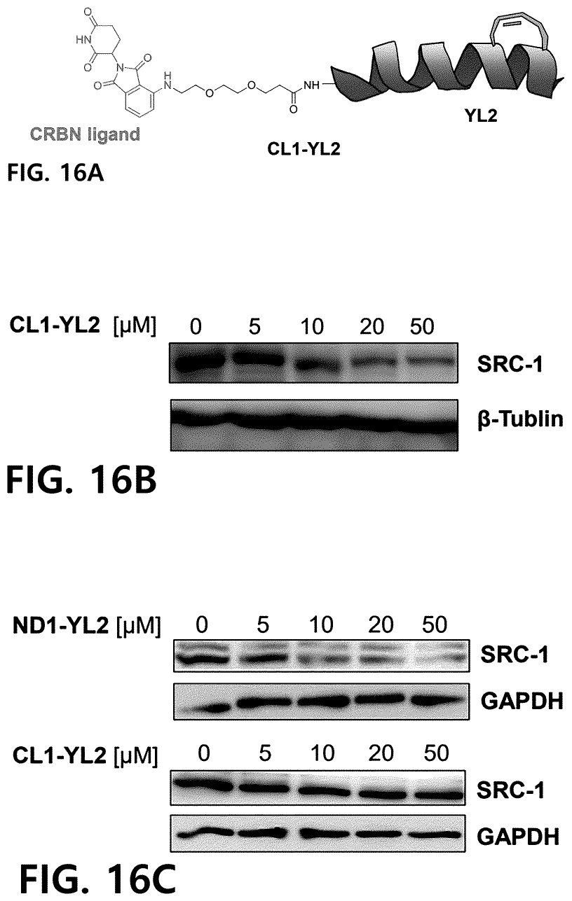

A competitive fluorescence polarization (FP) assay was performed to evaluate the binding affinity of ND1-YL2 to the target protein. A fluorescently labeled 15-mer STAT-6 peptide (100 nM) was cultured with the PAS-B domain (5 μM) of SRC-1 in a binding buffer (50 mM HEPES, pH 7.4, 150 mM NaCl, and 0.01% Tween 20). After being placed in black Costar 384-well plates for 30 minutes, they were treated with various concentrations of YL2 and ND1-YL2. After further incubation for 1 hour, the polarization of fluorescence was measured by a Tecan F200 microplate reader (excitation wavelength: 485 nm; emission wavelength: 535 nm). IC₅₀ values were determined using non-linear regression and fitted with GraphPad Prism® 4 software using the following equation: Y=Bottom+(Top-Bottom)/(1+10^{X-LogIC50}) (FIG. 11A). For analysis of binding to the UBR box, 10 nM of fluorescein-labeled RLAA peptide known to bind to the UBR box was incubated with the UBR box (10 μM) in a binding buffer (50 mM Tris-HCl, pH 8.0, 50 mM NaCl, and 0.01% Tween 20). The binding affinity was calculated as described above (FIG. 11B). As shown in FIG. 16, the ND1-YL2 bound to the PAS-B domain of SRC-1 with a Kᵢ value of 320 nM, which was found to be similar to that of the original stapled peptide YL2 (Ki=140 nM). In addition, FP analysis was used to confirm whether ND1-YL2 had binding ability similar to that of the fluorescently labeled RLAA peptide from the UBR box. Compared to the RLAA tetrapeptide (Kᵢ=4.2 μM), it exhibited approximately 3-fold improved affinity for the UBR box (Kᵢ=1.48 μM), which appears to be the result of additional contact with the UBR box by the linker. This result indicates that ND1-YL2 can induce the formation of a ternary complex.

### Example 8

### Size Exclusion Chromatography Combined with Multiple Angle Light Scattering (SEC-MALS)

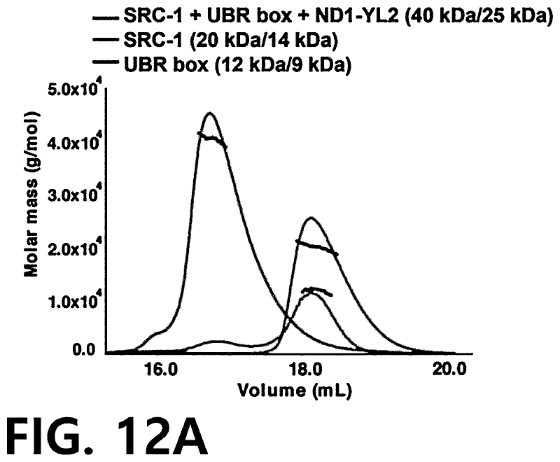

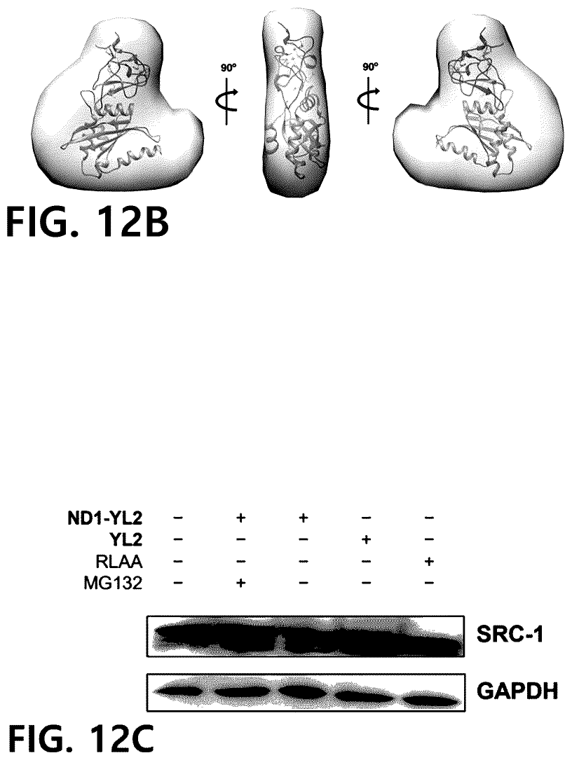

The next step was to confirm whether ND1-YL2 could indeed form a ternary complex. Size exclusion chromatography (SEC) was performed, showing that both SRC-1 and UBR box proteins were combined together in the presence of ND1-YL2, forming a ternary complex of SRC-1/ND1-YL2/UBR box. Protein samples (>2 mg/ml) were loaded into a Superdex™ 200 Increase 10/300 GL size exclusion chromatography column (GE Healthcare) using a fast protein liquid chromatography system (GE Healthcare) in 20 mM Tris-HCl pH 7.5, 150 mM NaCl and 1 mM TCEP at a rate of 0.5 ml per minute. The column outlet was fed to a DAWN®TREOS™ detector (Wyatt Technology) and then to an Optilab®rEX™ differential refractometer (Wyatt Technology). Light scattering and differential refractive index data were collected and analyzed using ASTRA®V software (Wyatt Technology) (FIG. 12A). The individual molecules SRC-1 and UBR box had molecular masses of 20 and 12 kDa, respectively, and were detected to be 40 kDa in the presence of ND1-YL2, which is a result showing that a ternary complex was formed (FIG. 12A). In addition, this result indicated that the SRC-1/ND1-YL2/UBR box formed a 1:1:1 ternary complex.

### Example 9

### Size Exclusion Chromatography Coupled Small Angle X-Ray Scattering (SEC-SAXS)

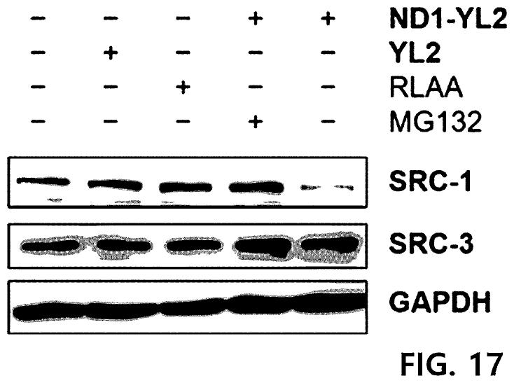

In order to more accurately confirm the results of Example 8, small-angle X-ray scattering (SAXS) was performed. To form a ternary complex, SRC-1 and UBR box were mixed with ND1-YL2 in a molar ratio of 1:1.3:1.2, respectively, and incubated at 4° C. for 4 hours. SEC-SAXS data were measured at beamline BL-10C of Photon Factory (Tsukuba, Japan). A protein sample of 5-10 mg/ml was injected into a Superdex™ 200 Increase 10/300 GL column (GE Healthcare, 28-9909-44), and a buffer of 50 mM Tris-HCl pH 7.5, 150 mM NaCl, and 1 mM TCEP was used. Data were collected using radiation at a wavelength of 1.5 Å with a detector of a PILATUS3 2M (DECTRIS) and a sample-to-detector distance of 1.0-m. X-ray scattering measurements were set at a frame rate of 20.001 sec (exposure time: 20 sec) at a flow rate of 0.1 ml/min. Data were normalized, averaged, and put on an absolute value for water without buffer according to standard operating procedures. The raw data from SEC-SAXS were processed by CHROMIXS (ATSAS program suite) and analyzed with the software package PRIMUS (ATSAS program suite) to determine radius of gyration (Rg), Porod volume and experimental molecular weight (J Appl Crystallogr 2012, 45, 342-350). An indirect Fourier transform of the scattering curve I(s) calculated by GNOM was used to obtain the distance distribution function P(r) and maximum particle size Dmax. (J Appl Crystallogr 1991, 24, 537-540). Ab initio modeling and averaging of these models were performed using DAMMIF. (J Appl Crystallogr 2009, 42, 342-346). The molecular envelope from the ab initio DAMMIF model was generated using chimeras. (J Comput Chem 2004, 25, 1605-1612). An atomic resolution structure of SRC-1 (PDB ID: [https://www.rcsb.org/structure/5Y7W]), UBR box (PDB ID: 3NIN [https://www.resb.org/structure/3NIN]) and GST (PDB ID: 1DUG [https ://www.rcsb.org/structure/1DUG]) was used as Chimera (https://www.cgl.ucsf.edu/chimera/docs/UsersGuide/midas/fitmap.html) to fit the model to the SAXS envelope (FIG. 17B). The analysis was performed using a high-resolution model of SRC-1 bound to YL2 (PDB ID: 5Y7W) and UBR box bound to RLGES (PDB ID: 3NIN). As shown in FIG. 12B, in the SRC-1/ND1-YL2/UBR box constituting the ternary complex, it can be seen that the ND1-YL2 brings the SRC-1 and the UBR box into close proximity, from which the ubiquitination is expected to occur.

### Example 10

### Confirmation of the Formation of Ternary Complexes in Cells by SRC-1 Degradation

To further confirm whether SRC-1 degradation is due to the formation of a ternary complex in cells, the cells were treated with either the stapled peptide itself (YL2) or a UBR-linked tetrapeptide (RLAA), and the cell level of SRC-1 were monitored by Western blot. MDA-MB-231 cells, breast cancer cells, were seeded at 5×10⁻⁵ cells per well in a 6-well plate (Corning). After incubation at 37° C. for 24 hours, the cells were treated with YL2, RLAA, or ND1-YL2 in Opti-MEM medium, and cultured for 12 hours. The cells were washed twice with cold Dulbecco's phosphate buffered saline (DPBS) prior to cell lysis. In order to lyse the cells, the cells on ice were treated with lysis buffer (50 mM Tris HCl pH 7.4, 150 mM NaCl, 1% TritonX, 1 mM EDTA, 1 mM DTT and 1×protease inhibitor cocktail). The cell lysate was centrifuged at 13,000×rpm for 15 minutes at 4° C. The supernatant was collected and the protein concentration was determined by Pierce™ 660 nm protein assay. 6×SDS loading buffer was added to the cell lysate, which was heated at 95° C. for 5 minutes. The same amount of protein was loaded on SDS-PAGE, which was transferred to a PVDF membrane. The membrane was blocked with 5% skim milk in TBST (tris buffered saline containing 0.01% Tween-20) and treated with a primary antibodys at 4° C. for 12 hours. After incubation of the HRP-conjugated secondary antibody at room temperature for 1 hour, Western blot images were obtained with ECL solution. Neither YL2 nor RLAA tetrapeptide per se had any effect on SRC-1 degradation. It was confirmed that the degradation of SRC-1 appeared only in ND1-YL2. These findings confirmed that proximity formation between SRC-1 and the UBR box by ND1-YL2 is important for SRC-1 degradation (FIG. 12C).

### Example 11

### Synthesis of CL1-YL2 to CL3-YL2, a Compound in which a Cerebron (CRBN) Ligand is Bound to YL2

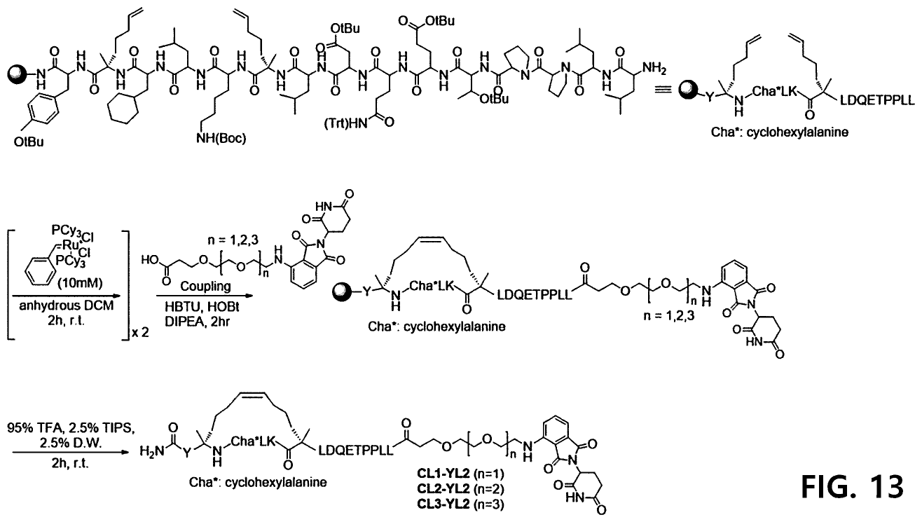

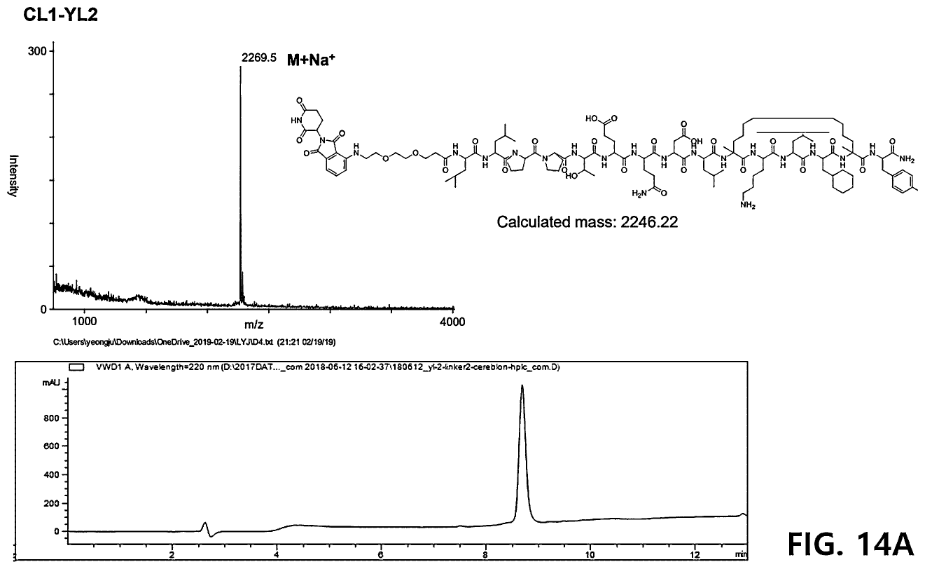

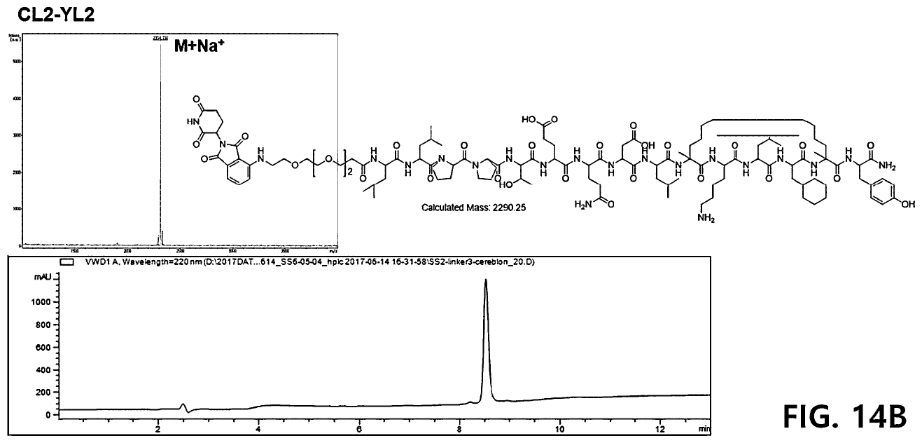

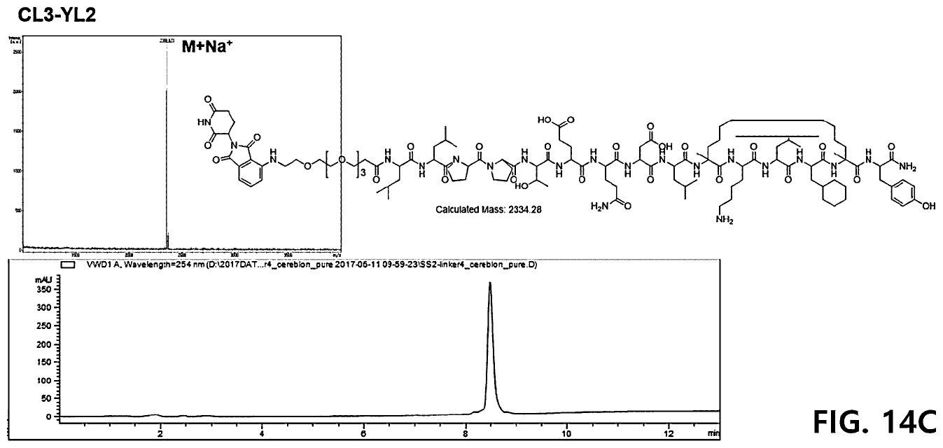

The efficacy of ND1-YL2 was compared with the activity of SRC-1 degrader designed using the conventional PROTAC method. To this end, in the present invention, a chimeric (PROTAC) molecule composed of pomalidomide (Kᵢ=156 nM), a potent small molecule ligand for YL2 and CRBN, was synthesized, wherein the pomalidomide is one of the most commonly used E3 ligase ligands in PROTAC. CL1-YL2, CL2-YL2, and CL3-YL2 were synthesized through a di(ethylene glycol) linker. A 15-residue peptide was synthesized using a similar procedure as in Example 1. After peptide coupling, the Fmoc group was removed from DMF with 20% piperidine. A CRBN ligand conjugated with the di(ethylene glycol) linker was bound to the N-terminus of the 15-residue peptide under the same peptide coupling conditions. The resulting peptide was cleaved from the resin using a cleavage cocktail at room temperature for 2 hours. The product was purified by HPLC (FIG. 13). The structures, MS and LC data of the purified CL1-YL2, CL2-YL2 and CL3-YL2 are as shown in FIGS. 14A to 14C.

### Example 12

### Confirmation of Target Protein Degradation of CL1-YL2 to CL3-YL2 Peptides

To evaluate the ability of the CL1-YL2 to CL3-YL2 peptides synthesized in Example 1 to degrade SRC-1 in cells, human triple negative breast cancer (TNBC) MDA-MB-231 cells were treated with DMSO or various concentrations of the synthesized CL1-YL2 to CL3-YL2 peptides. MDA-MB-231 cells were seeded at 5×10⁵ cells per well in a 6-well plate (Corning). After incubation at 37° C. for 24 hours, the cells were treated with the synthesized compounds in Opti-MEM medium. The cells were washed twice with cold Dulbecco's phosphate buffered saline (DPBS) prior to cell lysis. In order to lyse the cells, the cells on ice were treated with lysis buffer (50 mM Tris HCl pH 7.4, 150 mM NaCl, 1% TritonX, 1 mM EDTA, 1 mM DTT and 1×protease inhibitor cocktail). The cell lysate was centrifuged at 13,000×rpm for 15 minutes at 4° C. The supernatant was collected and the protein concentration was determined by Pierce™ 660 nm protein assay. 6×SDS loading buffer was added to the cell lysate, which was heated at 95° C. for 5 minutes. The same amount of protein was loaded on SDS-PAGE, which was transferred to a PVDF membrane. The PVDF membrane was blocked with 5% skim milk in TBST (tris buffered saline containing 0.01% Tween-20) and treated with a primary antibodys at 4° C. for 12 hours. After incubation of the HRP-conjugated secondary antibody at room temperature for 1 hour, Western blot images were obtained with ECL solution. A total of three peptides of CL1-YL2 to CL3-YL2 were treated, and SRC-1 degradation was observed when several of them were treated (FIG. 15). Among the tested compounds, CL1-YL2 reduced an expression level of SRC-1 at the cellular level in a dose-dependent manner with a DC₅₀ of ˜10 μM, and showed the strongest activity among the synthesized and treated compounds (FIG. 15).

### Example 13

### Comparison of SRC-1 Proteolytic Activity of ND1-YL2 and CL1-YL2 Peptides

In order to compare the SRC-1 proteolytic activity of ND1-YL2 and CL1-YL2 peptides in cells, A549 cells, which are human lung carcinoma cells, were treated with various concentrations of CL1-YL2, and the effect on SRC-1 degradation was analyzed by Western blot. A549 cells were seeded at 5×10⁵ cells per well in a 6-well plate (Corning). Colo205 cells were seeded at 6×10⁵ cells per well in a 6-well plate. After incubation at 37° C. for 24 hours, the cells were treated with the synthesized compounds in Opti-MEM medium. The cells were washed twice with cold Dulbecco's phosphate buffered saline (DPBS) prior to cell lysis. In order to lyse the cells, the cells on ice were treated with lysis buffer (50 mM Tris HCl pH 7.4, 150 mM NaCl, 1% TritonX, 1 mM EDTA, 1 mM DTT and 1×protease inhibitor cocktail). The cell lysate was centrifuged at 13,000×rpm for 15 minutes at 4° C. The supernatant was collected and the protein concentration was determined by Pierce™ 660 nm protein assay. 6×SDS loading buffer was added to the cell lysate, which was heated at 95° C. for 5 minutes. The same amount of protein was loaded on SDS-PAGE, which was transferred to a PVDF membrane. The PVDF membrane was blocked with 5% skim milk in TBST (tris buffered saline containing 0.01% Tween-20) and treated with a primary antibodys at 4° C. for 12 hours. After incubation of the HRP-conjugated secondary antibody at room temperature for 1 hour, Western blot images were obtained with ECL solution. Compared to CL1-YL2, ND1-YL2 has a much less strong binding affinity for its target E3 ligase (approximately a 10-fold difference in their binding), but CL1-YL2 showed activity similar to that of ND1-YL2 in inducing SRC-1 degradation. This is thought to be caused by the catalytic properties of ND1-YL2 in proteolysis via the N-degron pathway and the high expression level of UBR protein in all cells (in the UBR protein, not a single protein performs this action, but several proteins belonging to the UBR family perform the degradation function). Since the UBR proteins are expressed non-ubiquitously across different cells, ND1-YL2 cannot be used in cells or tissues that do not express the E3 ligases targeted by most current PROTACs, and therefore can degrade cellular SRC-1 regardless of cell type. To confirm this, ND1-YL2 or CL1-YL2 were treated with human colon carcinoma Colo205 cells expressing very low levels of CRBN at various concentrations. ND1-YL2 indeed induced a decrease in the expression level of SRC-1 in a dose-dependent manner, whereas CL1-YL2 had no effect (FIG. 16C). This result indicates that PROTAC based on the N-degron pathway can be applied in various cells and can be a very useful and generally applicable tool for the degradation of targeted proteins in various diseases.

### Example 14

### Confirmation of Selective Proteolysis of ND1-YL2 Against SRC-1 Among SRC Family Members

It was confirmed whether ND1-YL2 could selectively target and degrade SRC-1 over other SRC family members. Considering the structural similarity between SRC members, it can be predicted that ND1-YL2 will be able to bind to and degrade other members of the SRC family, such as SRC-3. In addition, many inhibitors of SRC-1 protein are known to simultaneously inhibit the function of SRC-3. To test this, TNBC MDA-MB-231 cells were treated with ND1-YL2 and the amounts of SRC-3 and SRC-1 were confirmed at the cellular level by Western blotting (FIG. 17).

As a result, ND1-YL2 selectively degraded only SRC-1, and no degradation effect was observed on SRC-3 (FIG. 17). This result was consistent with previous studies showing that STAT-6 interacts with SRC-1 but not with SRC-3. That is, considering that the stapled peptide portion of ND1-YL2 is derived from the SRC-1 binding peptide motif of STAT-6, the resulting chimeric peptide ND1-YL2 does not bind to SRC-3 but binds specifically to SRC-1, thereby inducing selective degradation of SRC-1.

### Example 15

### Confirmation of Gene Regulation Related to Cell Migration and Invasion of ND1-YL2

The pharmacological effects on SRC-1 dependent signal transmission were explored using the previously discovered SRC-1 degrading substance. The SRC-1 is overexpressed in various cancers and plays a pivotal role in increasing cell migration and invasion by regulating the expression of related genes. Therefore, when treated with ND1-YL2, an SRC-1 degrading substance, the expression of genes stimulated by SRC-1, such as colony stimulating factor-1 (CSF-1), a gene encoding the cytokine CSF-1 that induces cell differentiation and migration, will be suppressed. To confirm this, MDA-MB-231 cells were treated with DMSO and various concentrations of ND1-YL2, YL2 or N-degron tetrapeptide for 12 hours, and the mRNA level of CSF-1 was normalized to 18S level. Confirmation was made using quantitative real-time polymerase chain reaction (RT-qPCR). MDA-MB-231 cells were seeded at 2×10⁵ cells per well. After 24 hours, compounds (YL2, RLAA or ND1-YL2) were treated in Opti-MEM medium for 12 hours. Subsequently, general mRNA isolation was performed using TRI reagent (Takara), and total mRNA was isolated from the cells. The absorbance of total mRNA was measured with a Tecan F200 Nanophotometer, and reverse transcription was performed using AccuPower RocketScript Cycle RT PreMix (Bioneer). Quantitative real-time PCR for CSF-1, E-Cadherin and 18S was performed using a StepOnePlus Real-Time PCR System and SYBR Green mix (Applied Biosystems) according to the manufacturer's manual.

Primer sequences for real-time PCR:

(a) forward primer for CSF-1
(SEQ ID NO: 18)
5′-GTT TGT AGA CCA GGA ACA GTT GAA-3′
and
reverse primer
(SEQ ID NO: 19)
5′-CGC ATG GTG TCC ATT AT-3′,
(b) forward primer for E-cadherin
(SEQ ID NO: 20)
5′-TGC TGC AGG TCT CCT CTT GG-3′
and
reverse primer
(SEQ ID NO: 21)
5′-AGT CCC AGG CGT AGA CCA AG-3′,
(c) 18S forward primer
(SEQ ID NO: 22)
5′-GAG GCC GTA GGC TTA TTG TG-3′
and
reverse primer
(SEQ ID NO: 23)
5′-GAG TAG CTC ATA TGT CTT CCC TAC CT-3′.

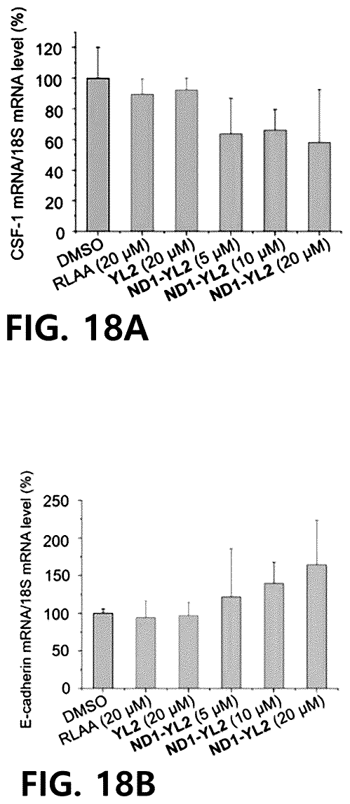

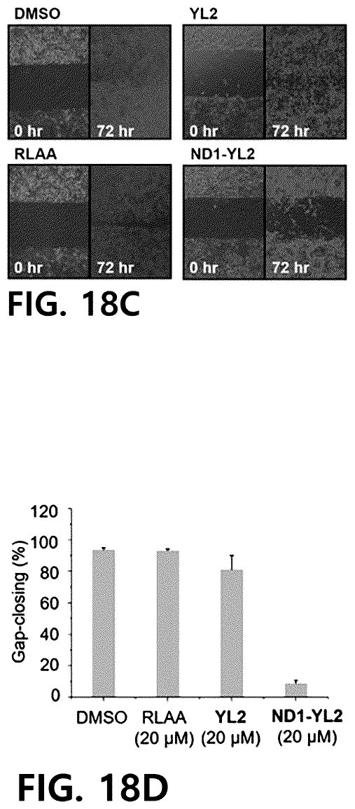

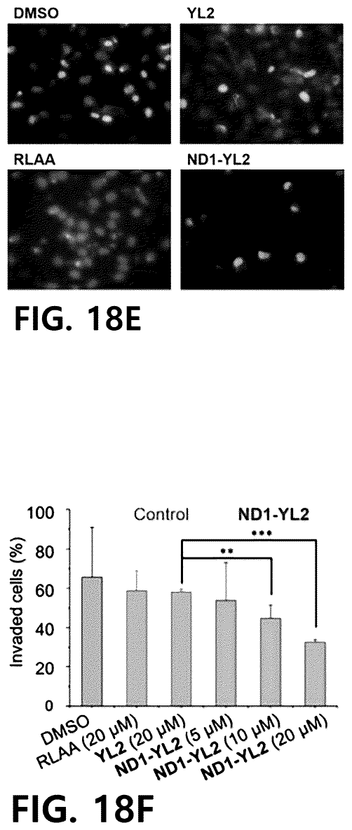

Indeed, The ND1-YL2 downregulated CSF-1 expression, whereas the YL2 and RLAA peptides did not significantly affect the CSF-1 expression. In addition, the effect of the ND1-YL2 on the expression of E-cadherin, a tumor suppressor gene that plays an important role in cell-cell adhesion, was tested. Since SRC-1 protein is known to suppress the E-cadherin expression, the ND1-YL2 will upregulate the E-cadherin by degrading SRC-1. Treatment with the ND1-YL2 resulted in a dose-dependent increase in the E-cadherin, whereas control compounds (YL2 and RLAA) had no effect (FIGS. 18A, 18B).

### Example 16

### Gap Closure Migration Assay on Cell Migration and Invasion Effects of ND1-YL2

Based on the results that the SRC-1 degradation effectively affects the transcription mediated by SRC-1 (FIGS. 18A and 18B), it was predicted that ND1-YL2 would exert an inhibitory effect on downstream signaling pathways such as increased cancer cell migration and invasion. To test this, we first performed a gap closure migration assay using an invasive MDA-MB-231 cell line. MDA-MB-231 cells (70 μL of 4×10⁵ cells/mL) were seeded in culture insert 2 wells (ibidi, 80209) pre-inserted into p-dishes (ibidi) under the conditions of 37° C. and 5% CO₂. The culture insert 2 wells was gently removed with sterile tweezers. The cells were treated with DMSO, YL2 (20 04), RLAA (20 μM) or ND1-YL2 (20 μM) in Opti-MEM medium for 72 hours. Images were obtained by a camera (Olympus) attached to an optical microscope. The cells were grown in the culture insert wells, and the inserts were removed to create intercellular gaps inside the wells. Subsequently, after treated with DMSO, YL2, RLAA peptide or ND1-YL2, the cells were cultured for 72 hours and the degree of cell migration was observed. The cell migration was quantified by analyzing the images of cells filling the gap. As shown in FIGS. 18C and 18D, The ND1-YL2 blocked the cell migration by maintaining the existing gaps at about 80%, whereas the cell migration was not blocked in cells treated with YL2 or RLAA peptides, and thus the gaps were completely filled. This result demonstrated that SRC-1 degradation caused by the ND1-YL2 inhibits SRC-1 mediated cell migration.

### Example 17

### Invasion Assay

The inhibition of cell migration may be due to the inhibitory effect of ND1-YL2 on cell growth. In order to exclude this possibility, the invasive ability of cells treated with ND1-YL2 was confirmed. A Corning Matrigel insert (Corning) was placed in a 24-well plate. After hydrating the Matrigel insert with an FBS-free DMEM medium for 2 hours in a CO₂ incubator, MDA-MB-231 cells were cultured in the Matrigel insert (4×10⁴ cells/insert) with 125 μL of Opti-MEM medium. Another 125 μl of compound containing Opti-MEM medium was added to the insert. The lower chamber was filled with 500 μL of DMEM medium containing 10% FBS. The Matrigel chamber was cultured for 24 hours under the conditions of 37° C. and 5% CO₂. The cell culture media of the upper and lower chambers were removed and washed three times with cold DPBS. Non-invasive cells were then removed with a cotton swab. The bottom of the chamber was fixed with 4% formaldehyde for 10 minutes at room temperature, and stained with Hoechst 33342 (5 μg/mL) for 5 minutes in a CO₂ incubator. After washed with DPBS, images of infiltrated cells were obtained by a fluorescence microscope. The percentage of the invasive cells was calculated using the product manual. MDA-MB-231 cells were treated with various concentrations of ND1-YL2, N-degron (20 μM) or YL2 (20 μM). After 24 hours, images of the invaded cells were obtained by a fluorescence microscope, and the number of invaded cells was quantified through the images. This result was consistent with the cell migration experiments (FIGS. 18E an 18F). Although the cell invasion decreased as the treatment concentration of ND1-YL2 increased, no significant effect was observed for YL2 or RLAA peptides. The inhibitory activity of ND1-YL2 on cell migration and invasion is in good agreement with previous SRC-1 knockdown experiments.

### Example 18

### Cell Viability Assay

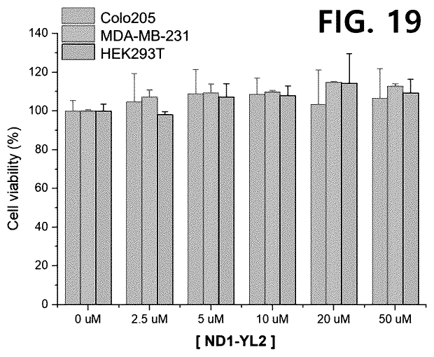

To further confirm the above results, an MTT cell viability assay was performed. MDA-MB-231 (1×10⁴ cells/well), HEK293T (1×10⁴ cells/well) and Colo205 (2×10⁴ cells/well) cells were seeded in a 96-well plate, and cultured under the conditions of 37° C. and 5% carbon dioxide. After 24 hours, the cells were washed twice with DPBS and treated with DMSO or various concentrations of ND1-YL2 in Opti-MEM medium for 48 hours. Cell viability was measured by the Cyto X cell viability assay kit (LPS solution) according to the manufacturer's instructions. The results were consistent with previous results when SRC-1 was knocked down using siRNA, and SRC-1 degradation by ND1-YL2 did not significantly affect the survival of various cell lines (FIG. 19). Taking these results together, the strategy of chemically degrading SRC-1 by ND1-YL2 is expected to be an effective means for inhibiting cancer cell migration and invasion.

### Example 19

### Confirmation of Anticancer Effect of ND1-YL2 in In Vivo Mice Model

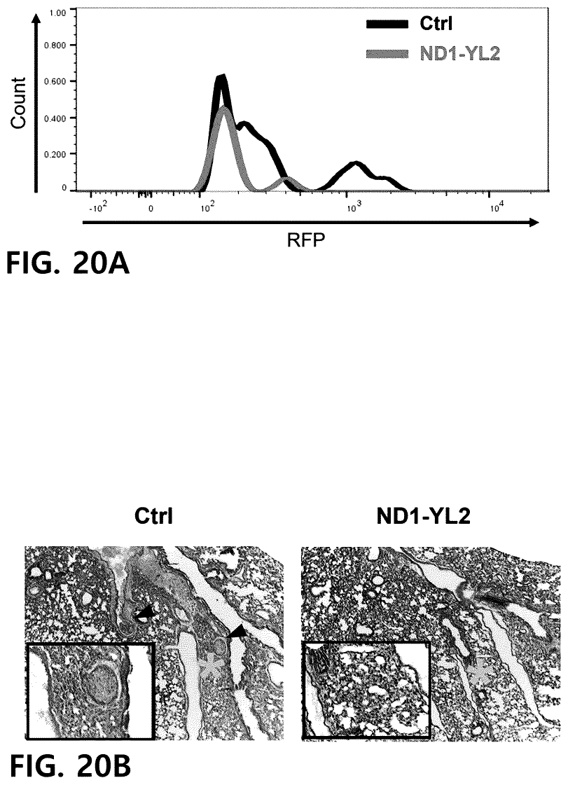

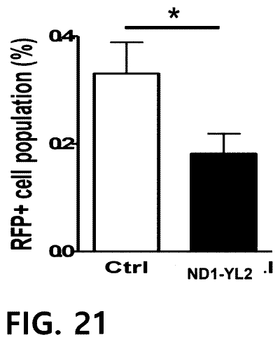

It was confirmed whether ND1-YL2 could inhibit cancer metastasis through mice model. MDA-MB-231-RFP cells expressing red fluorescent protein (RFP) were cultured in DMEM (Welgene) containing 10% FBS (Omega Scientific Inc.) under the conditions of 37° C. and 5% CO₂. 5-week-old BALB/c-nude mice (OriententBio Inc.) were maintained in a sterile environment and had free access to food and water. All animal procedures were approved by the Organ Animal Care and Use Committee of POSTECH. For the pulmonary metastasis model, MDA-MB-231-RFP cells treated with DMSO or 100 μM of ND1-YL2 for 12 hours were intravenously injected at 10⁶ cells per mouse, followed by lung harvesting for two weeks. The lungs were harvested and prepared in a single cell suspension and filtered through a 100 μm cell strainer (Corning). It was further cultured for 2 minutes in ACK lysis buffer (Gibco). The samples were analyzed by LSR Fortessa (BD Biosciences). Upon treatment with ND1-YL2, infiltrated MDA-MB-231-RFP cells were significantly reduced by 40% compared to vehicle samples (FIG. 20). To visualize lung metastasis, lung sections were prepared and dyed with Haemotoxylin and Eosin (H&E) stain. Metastatic tumors were found only in the lung area of mice injected with DMSO-treated MDA-MB-231 cells, but not in samples treated with ND1-YL2 (FIG. 21). These results indicate that the ND1-YL2 effectively degrades SRC-1 and inhibits metastasis of breast cancer cells in vivo.

1. A chimeric compound having a structure of Formula 1 below: 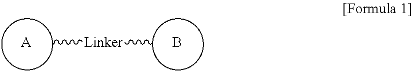
wherein:
A represents a ubiquitin ligase binding moiety (ULM) that binds to any one or more E3 ubiquitin ligases selected from the group consisting of ubiquitin-protein ligase E3 component n-recognin 1 (UBR1), ubiquitin-protein ligase E3 component n-recognin 2 (UBR2) and ubiquitin-protein ligase E3 component n-recognin 4 (UBR4), and
B represents a protein target moiety (PTM) that binds to steroid receptor coactivator-1 (SRC-1), wherein A and B are chemically linked by a linker.

2. The chimeric compound according to claim 1, wherein the chimeric compound binds simultaneously to protein and ubiquitin ligase and the protein is ubiquitinated by the ubiquitin ligase.

3. The chimeric compound according to claim 1, wherein the linker has a structure of Formula 2:
—Y₁—Y₂—Y₃— [Formula 2]
wherein Y₁ is R₁, or Y₁ is absent;
R₁ is selected from the group consisting of —C(═O)N(H)—, —N(H)—, —N(H)C(═O)—, —O—, —CH₂—, —CH═CH— and —C≡C—;
Y₂ is any one selected from the group consisting of —C(═O)N(H)—, —N(H)—, —N(H)C(═O)—, —O—, —CH₂—, —CH═CH— and —C≡C—; and
Y₃ is selected from the group consisting of —C(═O)—, —N(H)—, —C(═O)N(H)—, —N(H)C(═O)—, —O—, —CH₂—, —CH═CH— and —C≡C—, or absent.

4. The chimeric compound according to claim 3, wherein Y₂ is selected from the group consisting of —CH₂ (CH₂OCH₂)m1CH₂—, —(CH₂)m2—W—(CH₂)m3—, —(CH₂)m2—W—(CH₂)m4—O—(CH₂)m5— and —(N(H)CH(CH₃)C(═O))m6—;
W is selected from the group consisting of phenylene, five-membered heteroarene and cycloalkylene, or absent;
m1 is 1, 2, 3, 4, 5, 6 or 7;
m2is 0, 1, 2, 3, 4, 5, 6 or7;
m3 is 0, 1, 2, 3, 4, 5, 6 or 7;
m4 is 0, 1, 2, 3 or 4;
m5 is 0, 1, 2, 3 or 4; and
m6 is 0, 1, 2, 3 or 4.

5. The chimeric compound according to claim 1, wherein the protein target moiety (PTM) comprises an amino acid sequence of X₁X₂X₃X₄X₅X₆X₇X₈X₉X₁₀X₁₁X₁₂X₁₃X₁₄X₁₅ (SEQ ID NO: 1),
wherein the amino acid sequence of SEQ ID NO: 1 is a stapled peptide in which two amino acids in the amino acid sequence of SEQ ID NO: 1 are linked to each other, and in the amino acid sequence,
X₁, X₂, X₉ and X₁₂ are valine (V), alanine (A), isoleucine (I), leucine (L), norleucine (Nleu), 3-methyl valine or norvaline;
X₃ and X₄ are proline (P), hydroxy proline, amino proline, propynyl proline, chloro proline, bromo proline or trifluoromethyl proline;
X₅ is threonine (T), serine (S), homoserine, methyl homoserine, or alanine (A);
X₆ is glutamic acid (E), aspartic acid (D), or alanine (A);
X₇ is glutamine (Q), asparagine (N), or alanine (A);
X₈ is glutamic acid (E) or aspartic acid (D);
X₁₀ is (S)-2-(4′-pentenyl) alanine, cysteine (C), homocysteine, lysine (K), ornithine (Orn) or diaminobutyric acid (Dab);
X₁₁ is arginine (R), lysine (K) or alanine (A);
X₁₂ is leucine (L) or alanine (A);
X₁₃ is cyclohexylalanine (Cha), cyclopentylalanine (Cpa), cycloheptylpropanoic acid, phenylalanine (F), leucine (L), alanine (A), isoleucine (I) or valine (V);
X₁₄ is (S)-2-(4′-pentenyl) alanine, cysteine (C), homocysteine, lysine (K), ornithine (Orn) or diaminobutyric acid (Dab); and
X₁₅ is tyrosine (Y), serine (S), threonine (T) or alanine (A).

6. The chimeric compound according to claim 5, wherein two amino acids functionalized with a compound containing the (S)-2-(4′-pentenyl) alanine group is linked by a ring produced through ring-closing metathesis, or linked by a ring produced through ring-closing metathesis and then linked by a carbon-carbon single bond through a reduction reaction.

7. The chimeric compound according to claim 5, wherein two amino acids in the amino acid sequence of SEQ ID NO: 1 are X₁₀ and X₁₄ and the two amino acids are cysteine or homocysteine, respectively and they are linked by cyclization with a compound comprising a phenyl group.

8. The chimeric compound according to claim 7, wherein the compound comprising a phenyl group is represented by Formula 3 or Formula 4 below: 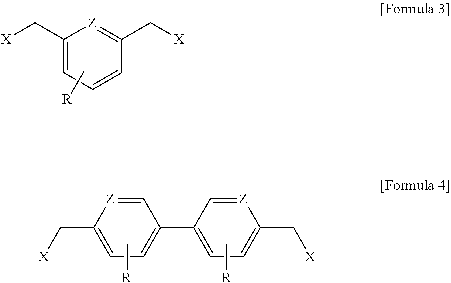
wherein X is at least one selected from the group consisting of chloro, bromo, and iodo; Z is nitrogen or oxygen; and R is at least one selected from the group consisting of hydrogen, halogen, C₁₋₄ alkyl, C₁₋₄ alkyl substituted with halogen, nitro, amino, and C₁₋₄ alkylamino.

9. The chimeric compound according to claim 5, wherein two amino acids in the amino acid sequence of SEQ ID NO: 1 are X₁₀ and X₁₄ and the two amino acids are lysine (K), ornithine (Orn) or diaminobutyric acid (Dab), respectively, and they are linked by cyclization with a compound comprising triazine.

10. The chimeric compound according to claim 5, wherein a linker is coupled to the N-terminus or C-terminus of SEQ ID NO: 1.

11. The chimeric compound according to claim 1, wherein the ubiquitin ligase binding moiety (ULM) comprises an amino acid sequence of X₂₀X₂₁X₂₂X₂₃ (SEQ ID NO: 16):

12. The chimeric compound according to claim 11, wherein
X₂₀ is arginine (R), histidine (H), lysine (K), phenylalanine (F), tyrosine (Y), isoleucine (I), tryptophan (W), glutamic acid (E) or aspartic acid (D);
X₂₁ is arginine (R), leucine (L), isoleucine (I), alanine (A), valine (V), glycine (G) or phenylalanine (F), or absent;
X₂₂ and X₂₃ are alanine (A), glycine (G) or valine (V), or absent.

13. The chimeric compound according to claim 1, wherein the compound comprises one or more selected from the group consisting of a plurality of ULMs, a plurality of PTMs, and a plurality of linkers.

14. The chimeric compound according to claim 1, wherein Formula 1 is any one formula selected from the group consisting of Formulas 5 to 16 below:

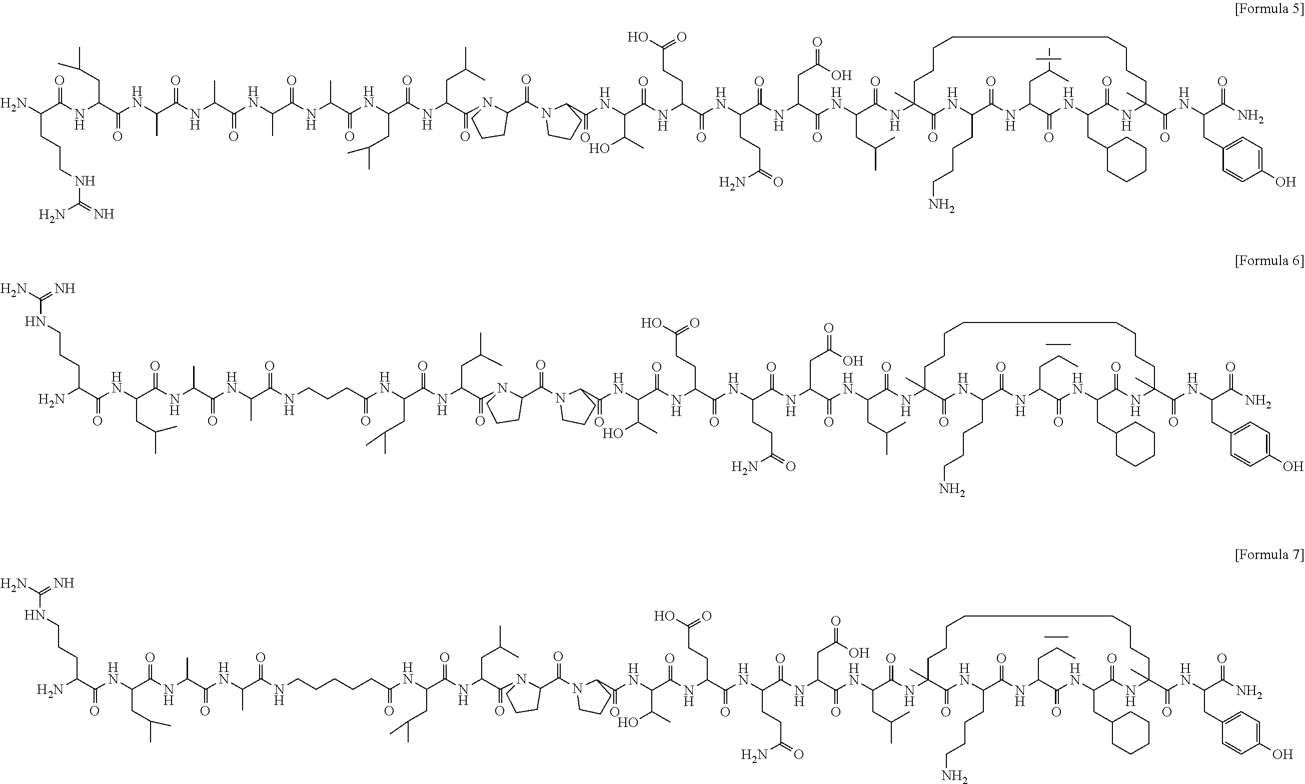

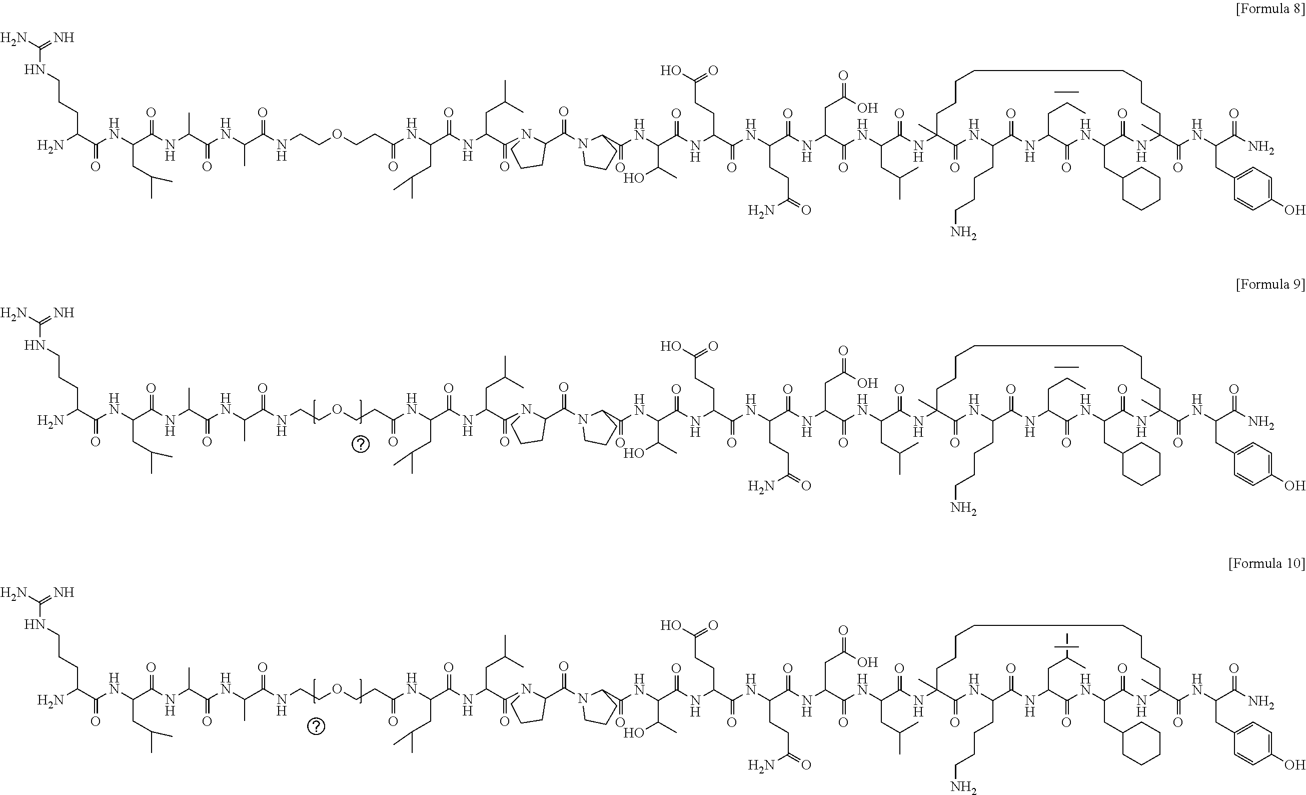

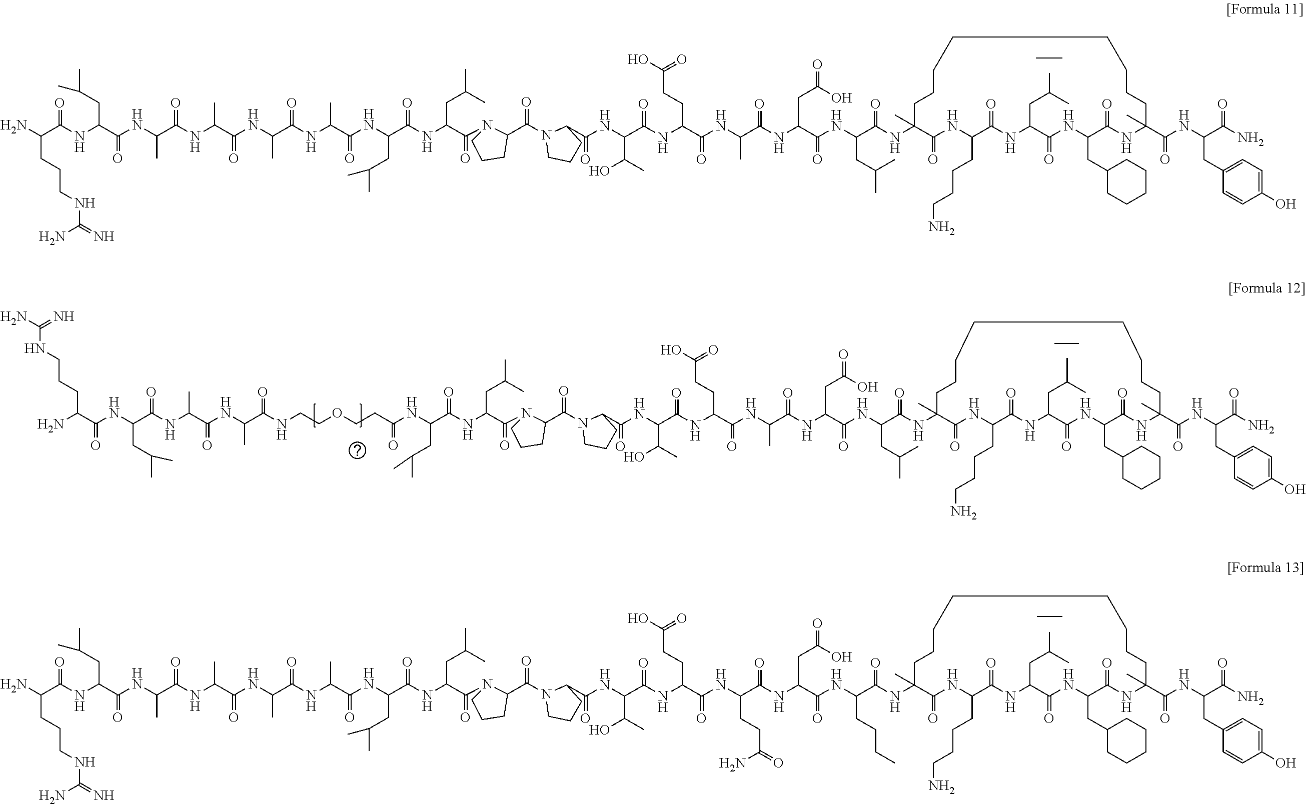

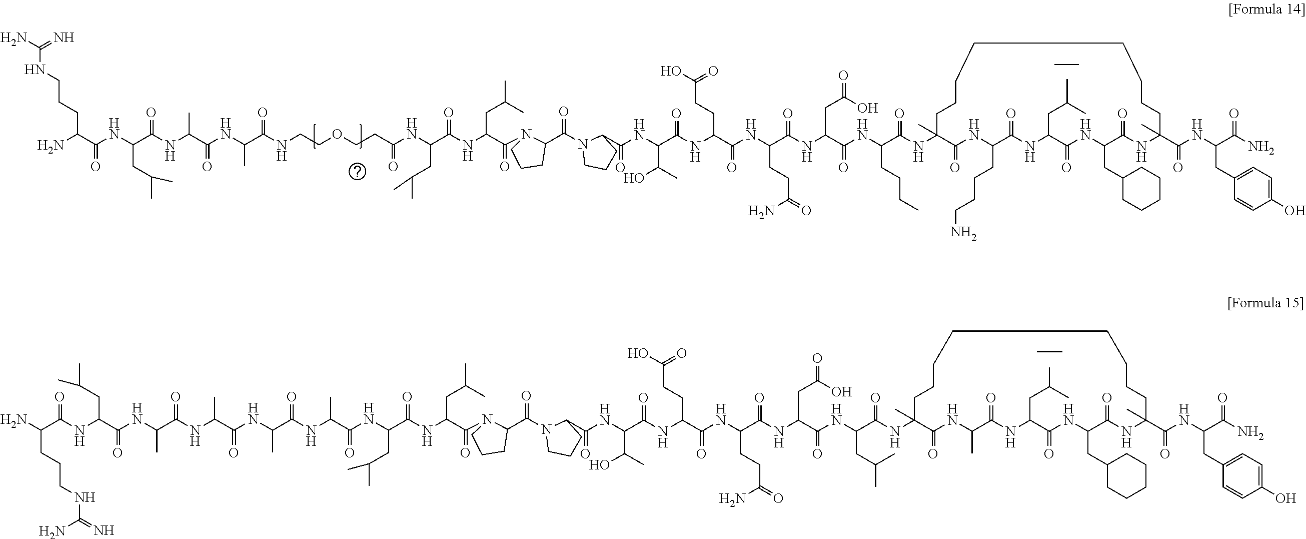

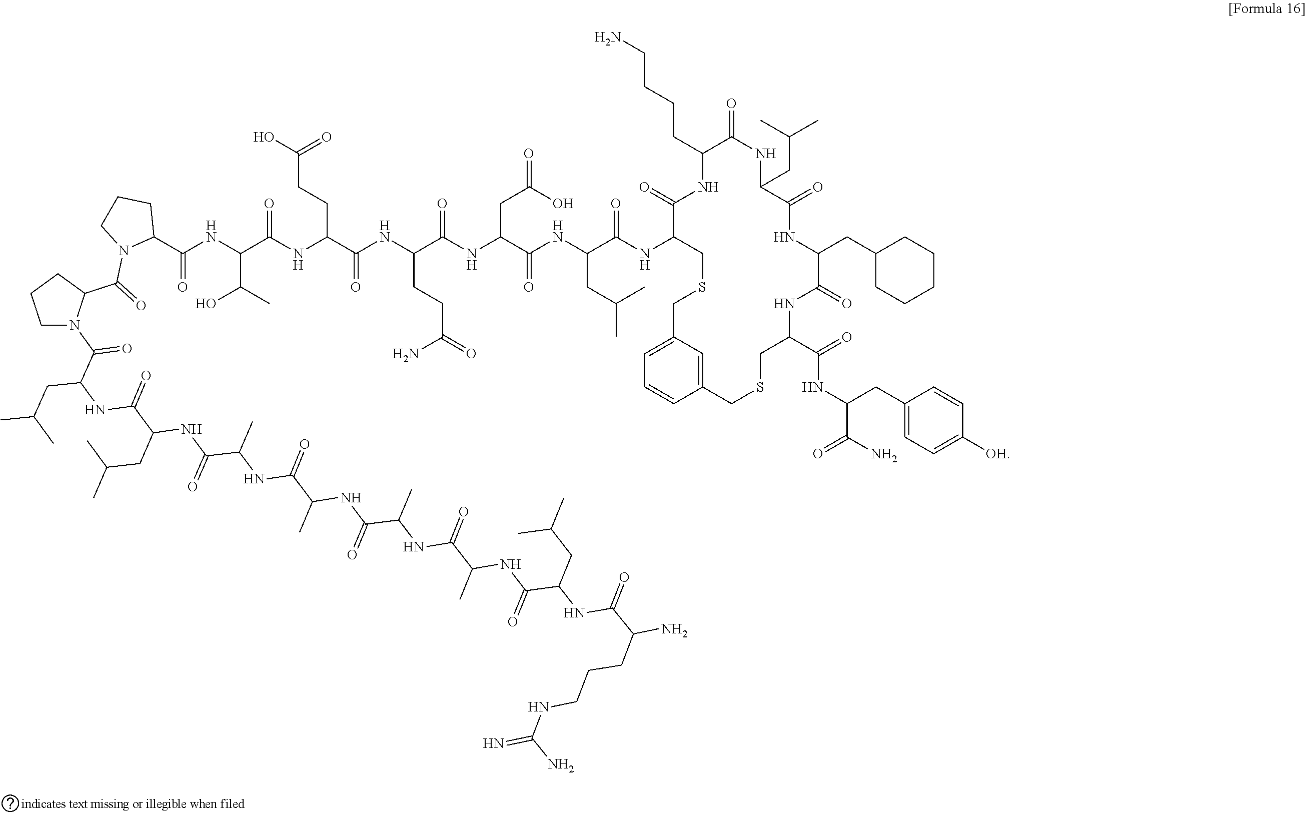 [SMILES: CC(C)CC(NC(=O)C(N)CCCNC(=N)N)C(=O)NC(C)C(=O)NC(C)C(=O)NC(C)C(=O)NC(C)C(=O)NC(CC(C)C)C(=O)NC(CC(C)C)C(=O)N1CCCC1C(=O)N1CCCC1C(=O)NC(C(=O)NC(CCC(=O)O)C(=O)NC(CCC(N)=O)C(=O)NC(CC(=O)O)C(=O)NC(CC(C)C)C(=O)NC1CSCc2cccc(c2)CSCC(C(=O)NC(Cc2ccc(O)cc2)C(N)=O)NC(=O)C(CC2CCCCC2)NC(=O)C(CC(C)C)NC(=O)C(CCCCN)NC1=O)C(C)O]

15. A method for preventing or treating diseases caused by overexpression of SRC-1, the method comprising administering the chimeric compound of claim 1, isomer, solvate or hydrate thereof to a subject in need thereof.

16. The method according to claim 15, wherein the disease is:
any one or more immune-related diseases selected from the group consisting of atopic dermatitis, asthma, airway hypersensitivity and chronic obstructive pulmonary disease;
any one or more selected from the group consisting of breast cancer, prostate cancer, skin melanoma, thyroid cancer, and endometrial cancer; or
metastasis of the cancer.
